(19) United States
(12) Patent Application Publication
(10) Pub. No.: US 2026/0090199 A1
(43) Pub. Date: Mar. 26, 2026

(54) COMPOUND AND ORGANIC LIGHT EMITTING DEVICE COMPRISING THE SAME

(71) Applicant: LG CHEM, LTD., Seoul (KR)

(72) Inventors: Dong Uk HEO, Seoul (KR); Hanna KIM, Seoul (KR); Jongjin PARK, Seoul (KR); Jung Min YOON, Seoul (KR); Jae Tak LEE, Seoul (KR); Su Jin HAN, Seoul (KR); Jun YUN, Seoul (KR); Sung Kil HONG, Seoul (KR)

(21) Appl. No.: 19/325,089

(22) Filed: Sep. 10, 2025

(30) Foreign Application Priority Data
Sep. 26, 2024 (KR) 10-2024-0130723
Aug. 25, 2025 (KR) 10-2025-0118231

(51) Int. Cl. H10K 50/16; C07D 251/14; C09K 11/06; H10K 50/17; H10K 85/60

(52) U.S. Cl. CPC H10K 50/16; C07D 251/14; C09K 11/06; H10K 50/171; H10K 85/622; H10K 85/626; H10K 85/633; H10K 85/654; H10K 85/6572; C09K 2211/1074

(57) ABSTRACT

A compound of Chemical Formula 1:

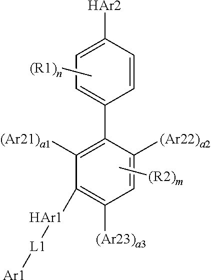

wherein: Ar1 is an aryl group having 6 to 60 carbon atoms that is substituted with one or more cyano groups, and is optionally further substituted with deuterium; Ar21 to Ar23 are the same as or different from each other, and are each independently hydrogen, deuterium, or a substituted or unsubstituted aryl group having 6 to 60 carbon atoms; and the other substituents are as defined in the specification; and an organic light emitting device including the same. The compound can be used as a material for an organic material layer of an organic light emitting device, and can be used as a material for a light emitting layer. An organic light emitting device including the compound of Chemical Formula 1 exhibits a low driving voltage, a high efficiency and/or a long service life compared to existing organic light emitting devices.

### CROSS-REFERENCE TO RELATED APPLICATIONS

This application claims priority to and the benefit of Korean Patent Application No. 10-2024-0130723 filed in the Korean Intellectual Property Office on Sep. 26, 2024, and Korean Patent Application No. 10-2025-0118231, filed in the Korean Intellectual Property Office on Aug. 25, 2025, the entire contents of which are incorporated herein by reference.

### TECHNICAL FIELD

The present specification relates to a compound and an organic light emitting device including the same.

### BACKGROUND ART

In the present specification, an organic light emitting device is a light emitting device using an organic semiconductor material, and requires an exchange of holes and/or electrons between electrodes and organic semiconductor materials. The organic light emitting device may be roughly divided into the following two organic light emitting devices depending on the operation principle. The first organic light emitting device is a light emitting device in which an exciton is formed in an organic material layer by a photon that flows from an external light source to the device, the exciton is separated into electrons and holes, and the electrons and the holes are each transferred to different electrodes and used as a current source (voltage source). The second organic light emitting device is a light emitting device in which holes and/or electrons are injected into organic semiconductor material layers forming an interface with an electrode by applying a voltage or current to two or more electrodes, and the device is operated by the injected electrons and holes.

An organic light emission phenomenon generally refers to a phenomenon converting electrical energy to light energy using an organic material. An organic light emitting device using the organic light emitting phenomenon usually has a structure including an anode, a cathode, and an organic material layer interposed therebetween. Here, the organic material layer has in many cases a multi-layered structure composed of different materials in order to improve the efficiency and stability of the organic light emitting device, and for example, may be composed of a hole injection layer, a hole transport layer, a light emitting layer, an electron blocking layer, an electron transport layer, an electron injection layer, and the like. In the structure of the organic light emitting device, if a voltage is applied between two electrodes, holes are injected from an anode into the organic material layer and electrons are injected from a cathode into the organic material layer, and when the injected holes and electrons meet each other, an exciton is formed, and light is emitted when the exciton falls down again to a ground state. Such an organic light emitting device has been known to have characteristics such as self-emission, high luminance, high efficiency, a low driving voltage, a wide viewing angle, and high contrast.

In an organic light emitting device, materials used as an organic material layer may be classified into a light emitting material and a charge transport material, for example, a hole injection material, a hole transport material, an electron blocking material, an electron transport material, an electron injection material, and the like depending on the function. The light emitting materials include blue, green, and red light emitting materials according to the light emitting color, and yellow and orange light emitting materials required for implementing a much better natural color.

Furthermore, a host/dopant system may be used as a light emitting material for the purpose of enhancing color purity and light emitting efficiency through energy transfer. The principle is that when a small amount of dopant which has a smaller energy band gap and better light emitting efficiency than those of a host mainly constituting a light emitting layer is mixed in the light emitting layer, the excitons generated by the host are transported to the dopant to emit light with high efficiency. In this case, it is possible to obtain light with a desired wavelength according to the type of dopant used because the wavelength of the host moves to the wavelength range of the dopant.

In order to fully exhibit the above-described excellent characteristics of the organic light emitting device, a material constituting an organic material layer in a device, for example, a hole injection material, a hole transport material, a light emitting material, an electron blocking material, an electron transport material, an electron injection material, and the like need to be supported by stable and efficient materials, so that there is a continuous need for developing a new material.

### RELATED ART DOCUMENTS

### Patent Documents

(Patent Document 1) International Patent Publication No. 2017-126443

### SUMMARY OF THE INVENTION

The present invention has been made in an effort to provide a compound and an organic light emitting device including the same.

An exemplary embodiment of the present invention provides An exemplary embodiment of the present specification provides a compound of the following Chemical Formula 1.

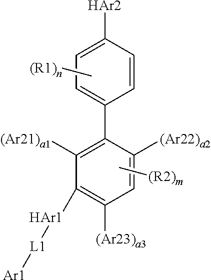

In Chemical Formula 1,

HAr1 is a divalent triazinyl group that is unsubstituted or substituted with one substituent selected from the group consisting of deuterium, a substituted or unsubstituted aryl group having 6 to 60 carbon atoms, and a substituted or unsubstituted heteroaryl group having 2 to 60 carbon atoms; a divalent pyrimidinyl group that is unsubstituted or substituted with one or two substituents each independently selected from the group consisting of deuterium, a substituted or unsubstituted alkyl group having 1 to 60 carbon atoms, a substituted or unsubstituted aryl group having 6 to 60 carbon atoms, and a substituted or unsubstituted heteroaryl group having 2 to 60 carbon atoms; a divalent quinazolinyl group that is unsubstituted or substituted with deuterium; a divalent benzothienopyrimidinyl group that is unsubstituted or substituted with one substituent selected from the group consisting of deuterium, a substituted or unsubstituted aryl group having 6 to 60 carbon atoms, and a substituted or unsubstituted heteroaryl group having 2 to 60 carbon atoms; or a divalent benzofuropyrimidinyl group that is unsubstituted or substituted with one substituent selected from the group consisting of deuterium, a substituted or unsubstituted aryl group having 6 to 60 carbon atoms, and a substituted or unsubstituted heteroaryl group having 2 to 60 carbon atoms,

HAr2 is a triazinyl group that is unsubstituted or substituted with two substituents each independently selected from the group consisting of deuterium, a substituted or unsubstituted aryl group having 6 to 60 carbon atoms, and a substituted or unsubstituted heteroaryl group having 2 to 60 carbon atoms; a pyrimidinyl group that is unsubstituted or substituted with one to three substituents each independently selected from the group consisting of deuterium, a substituted or unsubstituted alkyl group having 1 to 60 carbon atoms, a substituted or unsubstituted aryl group having 6 to 60 carbon atoms, and a substituted or unsubstituted heteroaryl group having 2 to 60 carbon atoms; a quinazolinyl group that is unsubstituted or substituted with one substituent selected from the group consisting of deuterium, a substituted or unsubstituted aryl group having 6 to 60 carbon atoms, and a substituted or unsubstituted heteroaryl group having 2 to 60 carbon atoms; a benzothienopyrimidinyl group that is unsubstituted or substituted with one substituent selected from the group consisting of deuterium, a substituted or unsubstituted aryl group having 6 to 60 carbon atoms, and a substituted or unsubstituted heteroaryl group having 2 to 60 carbon atoms; or a benzofuropyrimidinyl group that is unsubstituted or substituted with one substituent selected from the group consisting of deuterium, a substituted or unsubstituted aryl group having 6 to 60 carbon atoms, and a substituted or unsubstituted heteroaryl group having 2 to 60 carbon atoms,
L1 is a substituted or unsubstituted arylene group having 6 to 60 carbon atoms,
Ar1 is an aryl group having 6 to 60 carbon atoms that is substituted with one or more cyano groups, and is optionally further substituted with deuterium,
Ar21 to Ar23 are the same as or different from each other, and are each independently hydrogen; deuterium; or a substituted or unsubstituted aryl group having 6 to 60 carbon atoms,
R1 and R2 are the same as or different from each other, and are each independently hydrogen; deuterium; or a substituted or unsubstituted alkyl group having 1 to 30 carbon atoms,
n is an integer from 1 to 4, and when n is 2 or higher, R1's are the same as or different from each other,
m is an integer from 1 to 3, and when m is 2 or higher, R2's are the same as or different from each other,
a1, a2, and a3 are each 0 or 1,

$$a 1 + a 2 + a 3 \geq 1 , and$$

$$a 1 + a 2 + a 3 + m = 4.$$

Another exemplary embodiment provides an organic light emitting device including: a first electrode; a second electrode; and an organic material layer having one or more layers provided between the first electrode and the second electrode, in which one or more layers of the organic material layer include the above-described compound.

The compound of the present invention can be used as a material for an organic material layer of an organic light emitting device. When an organic light emitting device is manufactured by including the compound of the present invention, an organic light emitting device having high efficiency, low voltage and long-service life characteristics can be obtained, and when the compound of the present invention is included in an electron transport layer of an organic light emitting device, an effect of transferring electrons is high due to high intramolecular polarization, so that an organic light emitting device having long-service life characteristics can be manufactured.

The organic light emitting device of the present invention is characterized by including all of the compounds represented by Chemical Formula 1. In this case, the compound of the present invention allowed the LUMO orbital energy to have a suitable energy barrier at a level of −2.8 eV to −3.2 eV by substituting a linker between two heterocyclic groups in the para and meta directions, high efficiency characteristics to be exhibited by increasing the planarity of the material to maximize the electron mobility and long service life characteristics to be maintained by substituting a cyano group having excellent polarizability.

### BRIEF DESCRIPTION OF THE DRAWINGS

FIGS. 1 to 3 each illustrate an example of the organic light emitting device according to the present invention.

### DETAILED DESCRIPTION

Hereinafter, the present specification will be described in more detail.

When one part “includes” one constituent element in the present specification, unless otherwise specifically described, this does not mean that another constituent element is excluded, but means that another constituent element may be further included.

When one member is disposed “on” another member in the present specification, this includes not only a case where the one member is brought into contact with another member, but also a case where still another member is present between the two members.

In the present specification, “dotted line (---)” means a position bonded to a chemical formula or a compound.

In the present specification, the deuterium substitution rate of a compound may be understood by a method of calculating the substitution rate based on the max. value of the distribution which molecular weights form at the end point of a reaction using thin-layer chromatography/mass spectrometry (TLC-MS) or a quantitative analysis method using NMR, and a method of adding DMF as an internal standard and calculating the D-substitution rate from the integrated amount of the total peak using the integration rate on 1H NMR.

In the present specification, “X % deuterated”, “X % degree of deuteration”, or “X % deuterium substitution rate” means that X % of the hydrogen atoms at substitutable positions in the corresponding structure are substituted with deuterium.

In the present specification, hydrogen that can be substituted with deuterium in the corresponding structure may be substituted with deuterium at more than 0% and 100% or less, 0.1% or more and 99.99% or less, or up to 100%. For example, when the corresponding structure is dibenzofuran, the dibenzofuran being “25% deuterated”, the “degree of deuteration 25%” of the dibenzofuran, or the “deuterium substitution rate 25%” of the dibenzofuran may mean that two of the eight hydrogen atoms at the substitutable position of the dibenzofuran are substituted with deuterium, and the “deuterium substitution rate 50%” may mean that four of the eight hydrogen atoms at the substitutable position of the dibenzofuran are substituted with deuterium.

The term “substitution” means that a hydrogen atom bonded to a carbon atom of a compound is changed into another substituent, and a position to be substituted is not limited as long as the position is a position at which the hydrogen atom is substituted, that is, a position at which the substituent may be substituted, and when two or more hydrogen atoms are substituted, the two or more substituents may be the same as or different from each other.

In the present invention, the term “substituted or unsubstituted” means being substituted with one or two or more substituents selected from the group consisting of deuterium; a halogen group; a cyano group (—CN); a nitro group; a hydroxyl group; an alkyl group; a cycloalkyl group; an alkoxy group; a phosphine oxide group; an aryloxy group; an alkylthioxy group; an arylthioxy group; an alkylsulfoxy group; an arylsulfoxy group; an alkenyl group; a silyl group; a boron group; an amine group; an aryl group; and a heterocyclic group, being substituted with a substituent to which two or more substituents among the exemplified substituents are linked, or having no substituent. For example, “the substituent to which two or more substituents are linked” may be a biphenyl group. That is, the biphenyl group may also be an aryl group, and may be interpreted as a substituent to which two phenyl groups are linked.

In the present specification, the term “substituted or unsubstituted” means being substituted with one or two or more substituents selected from the group consisting of deuterium; a halogen group; a cyano group; a silyl group; an alkoxy group; an aryloxy group; an alkyl group; an aryl group; and a heterocyclic group, being substituted with a substituent to which two or more substituents among the exemplified substituents are linked, or having no substituent.

In the present specification, the term “substituted or unsubstituted” means being substituted with one or two or more substituents selected from the group consisting of deuterium; an alkyl group; an aryl group; and a heterocyclic group, being substituted with a substituent to which two or more substituents among the exemplified substituents are linked, or having no substituent.

In the present specification, the fact that two or more substituents are linked indicates that hydrogen of any one substituent is linked to another substituent. For example, an isopropyl group and a phenyl group may be linked to each other to become a substituent of or

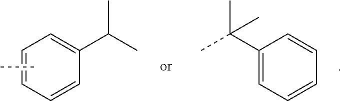

In the present specification, the fact that three substituents are linked to one another includes not only a case where (Substituent 1)-(Substituent 2)-(Substituent 3) are consecutively linked to one another, but also a case where (Substituent 2) and (Substituent 3) are linked to (Substituent 1). For example, two phenyl groups and an isopropyl group may be linked to each other to become a substituent of

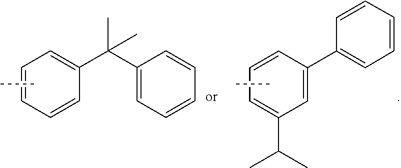

The same also applies to the case where four or more substituents are linked to one another.

Examples of the substituents will be described below; however, the substituents are not limited thereto.

In the present specification, examples of a halogen group include fluorine (—F), chlorine (—Cl), bromine (—Br) or iodine (—I).

In the present specification, a silyl group may be represented by a formula of —SiYaYbYc, and Ya, Yb, and Yc may be each hydrogen; a substituted or unsubstituted alkyl group; or a substituted or unsubstituted aryl group. Specific examples of the silyl group include a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group, and the like, but are not limited thereto.

In the present specification, a boron group may be represented by a formula of —BYdYe, and Yd and Ye may be each hydrogen; a substituted or unsubstituted alkyl group; or a substituted or unsubstituted aryl group. Specific examples of the boron group include a dimethylboron group, a diethylboron group, a t-butylmethylboron group, a diphenylboron group, a phenylboron group, and the like, but are not limited thereto.

In the present specification, the alkyl group may be straight-chained or branched, and the number of carbon atoms thereof is not particularly limited, but is preferably 1 to 60. According to an exemplary embodiment, the number of carbon atoms of the alkyl group is 1 to 30. According to another exemplary embodiment, the number of carbon atoms of the alkyl group is 1 to 20. According to still another exemplary embodiment, the number of carbon atoms of the alkyl group is 1 to 10. Specific examples of the alkyl group include a methyl group, an ethyl group, a propyl group, an n-propyl group, an isopropyl group, a butyl group, an n-butyl group, an isobutyl group, a tert-butyl group, a pentyl group, an n-pentyl group, a hexyl group, an n-hexyl group, a heptyl group, an n-heptyl group, an octyl group, an n-octyl group, and the like, but are not limited thereto.

In the present specification, the propyl group includes an n-propyl group and an isopropyl group.

In the present specification, the above-described description on the alkyl group may be applied to an arylalkyl group, except that the arylalkyl group is substituted with an aryl group.

In the present specification, the alkoxy group may be straight-chained, branched, or cyclic. The number of carbon atoms of the alkoxy group is not particularly limited, but is preferably 1 to 20. Specific examples thereof include methoxy, ethoxy, n-propoxy, isopropoxy, i-propyloxy, n-butoxy, isobutoxy, tert-butoxy, sec-butoxy, n-pentyloxy, neopentyloxy, isopentyloxy, n-hexyloxy, 3,3-dimethylbutyloxy, 2-ethylbutyloxy, n-octyloxy, n-nonyloxy, n-decyloxy, and the like, but are not limited thereto.

Substituents including an alkyl group, an alkoxy group, and other alkyl group moieties described in the present specification include both a straight-chained form and a branched form.

In the present specification, an alkenyl group may be straight-chained or branched, and the number of carbon atoms thereof is not particularly limited, but is preferably 2 to 40. According to an exemplary embodiment, the number of carbon atoms of the alkenyl group is 2 to 20. According to another exemplary embodiment, the number of carbon atoms of the alkenyl group is 2 to 10. According to still another exemplary embodiment, the number of carbon atoms of the alkenyl group is 2 to 6. Specific examples thereof include vinyl, 1-propenyl, isopropenyl, 1-butenyl, 2-butenyl, 3-butenyl, 1-pentenyl, 2-pentenyl, 3-pentenyl, 3-methyl-1-butenyl, 1,3-butadienyl, allyl, 1-phenylvinyl-1-yl, 2-phenylvinyl-1-yl, 2,2-diphenylvinyl-1-yl, 2-phenyl-2-(naphthyl-1-yl)vinyl-1-yl, 2,2-bis(diphenyl-1-yl)vinyl-1-yl, a stilbenyl group, a styrenyl group, and the like, but are not limited thereto.

In the present specification, the alkynyl group may be straight-chained or branched as a substituent including a triple bond between a carbon atom and a carbon atom, and the number of carbon atoms thereof is not particularly limited, but is preferably 2 to 40. According to an exemplary embodiment, the number of carbon atoms of the alkenyl group is 2 to 20. According to another exemplary embodiment, the number of carbon atoms of the alkenyl group is 2 to 10.

In the present specification, a cycloalkyl group is not particularly limited, but has preferably 3 to 60 carbon atoms, and according to an exemplary embodiment, the number of carbon atoms of the cycloalkyl group is 3 to 30. According to another exemplary embodiment, the number of carbon atoms of the cycloalkyl group is 3 to 20. According to another embodiment, the number of carbon atoms of the cycloalkyl group is from 3 to 6. Specific examples thereof may include a cyclopropyl group, a cyclobutyl group, a cyclopentyl group, a cyclohexyl group, a cycloheptyl group, a cyclooctyl group and the like, but are not limited thereto.

In the present specification, an amine group is —NH₂, and the amine group may be substituted with the above-described alkyl group, aryl group, heterocyclic group, alkenyl group, cycloalkyl group, a combination thereof, and the like. The number of carbon atoms of the substituted amine group is not particularly limited, but is preferably 1 to 30. According to an exemplary embodiment, the number of carbon atoms of the amine group is 1 to 20. According to an exemplary embodiment, the number of carbon atoms of the amine group is 1 to 10. Specific examples of the substituted amine group include a methylamine group, a dimethylamine group, an ethylamine group, a diethylamine group, a phenylamine group, a 9,9-dimethylfluorenylphenylamine group, a pyridylphenylamine group, a diphenylamine group, a phenylpyridylamine group, a naphthylamine group, a biphenylamine group, an anthracenylamine group, a dibenzofuranylphenylamine group, a 9-methylanthracenylamine group, a diphenylamine group, a phenylnaphthylamine group, a ditolylamine group, a phenyltolylamine group, a diphenylamine group, and the like, but are not limited thereto.

In the present specification, an aryl group is not particularly limited, but has preferably 6 to 60 carbon atoms, and may be a monocyclic aryl group or a polycyclic aryl group. According to an exemplary embodiment, the number of carbon atoms of the aryl group is from 6 to 30. According to an exemplary embodiment, the number of carbon atoms of the aryl group is from 6 to 20. The aryl group may be an aryl group composed of a single ring or a polycyclic aryl group (a bicyclic or more aryl group). The aryl group composed of the single ring may mean a phenyl group; or a group to which two or more phenyl groups are linked. Examples of the aryl group composed of the sing ring include a phenyl group, a biphenyl group, a terphenyl group, a quaterphenyl group, and the like, but are not limited thereto. The polycyclic aryl group may mean a group in which two or more monocyclic rings such as a naphthyl group and a phenanthrenyl group are fused. Examples of the polycyclic aryl group include a naphthyl group, an anthracenyl group, a phenanthrenyl group, a pyrenyl group, a perylenyl group, a chrysenyl group, a fluorenyl group, a triphenylenyl group, and the like, but are not limited thereto.

In the present specification, a fluorenyl group may be substituted, and two substituents may be bonded to each other to form a spiro structure.

When the fluorenyl group is substituted, the substituent may be a spirofluorenyl group such as

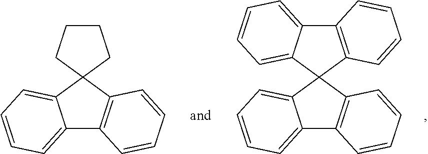

and a substituted fluorenyl group such as

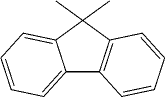

(a 9,9-dimethylfluorenyl group) and

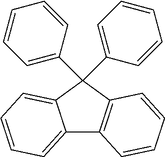 [SMILES: c1ccc(C2(c3ccccc3)c3ccccc3-c3ccccc32)cc1]

(a 9,9-diphenylfluorenyl group). However, the substituent is not limited thereto.

In the present specification, the above-described description on the aryl group may be applied to an aryl group in an aryloxy group.

In the present specification, a heterocyclic group is a cyclic group including one or more of N, O, P, S, Si, and Se as a heteroatom, and the number of carbon atoms thereof is not particularly limited, but is preferably 2 to 60. According to one embodiment, the number of carbon atoms of the heterocyclic group is from 2 to 30. According to one embodiment, the number of carbon atoms of the heterocyclic group is from 2 to 20. Examples of the heterocyclic group include a pyridine group, a pyrrole group, a pyrimidine group, a quinoline group, a pyridazinyl group, a furan group, a thiophene group, an imidazole group, a pyrazole group, a dibenzofuran group, a dibenzothiophene group, a carbazole group, a benzocarbazole group, a naphthobenzofuran group, a benzonaphthothiophene group, an indenocarbazole group, a triazinyl group, and the like, but are not limited thereto.

In the present specification, the above-described description on the heterocyclic group may be applied to a heteroaryl group except for an aromatic heteroaryl group.

In the present specification, a heteroaryl group includes one or more atoms other than carbon, that is, one or more heteroatoms, and specifically, the heteroatom may include one or more atoms selected from the group consisting of O, N, Se, S, and the like. The number of carbon atoms thereof is not particularly limited, but is preferably 2 to 30, and the heteroaryl group may be monocyclic or polycyclic. Examples of the heteroaryl group include a thiophene group, a furan group, a pyrrole group, an imidazole group, a thiazole group, an oxazole group, an oxadiazole group, a pyridine group, a bipyridine group, a pyrimidine group, a triazine group, a triazole group, an acridine group, a pyridazine group, a pyrazine group, a quinoline group, a quinazoline group, a quinoxaline group, a phthalazine group, a pyridopyrimidine group, a pyridopyrazine group, a pyrazinopyrazine group, an isoquinoline group, an indole group, a carbazole group, a benzoxazole group, a benzimidazole group, a benzothiazole group, a benzocarbazole group, a benzothiophene group, a dibenzothiophene group, a benzofuran group, a phenanthridine group, a phenanthroline group, an isoxazole group, a thiadiazole group, a dibenzofuran group, a dibenzosilole group, a phenoxathiine group, a phenoxazine group, a phenothiazine group, a dihydroindenocarbazole group, a spirofluorenexanthene group, a spirofluorenethioxanthene group, and the like, but are not limited thereto.

In the present specification, the description on the aryl group may be applied to an arylene group except for a divalent arylene group.

In the present specification, the description on the heterocyclic group may be applied to a divalent heterocyclic group except for a divalent heterocyclic group.

In the present specification, the description on the aryl group may be applied to a (n+1)-valent aryl group except for a (n+1)-valent aryl group.

In the present specification, the description on the heterocyclic group may be applied to a (n+1)-valent heterocyclic group except for a (n+1)-valent heterocyclic group.

In the present specification, in a substituted or unsubstituted ring formed by being bonded to an adjacent group, the “ring” means a hydrocarbon ring; or a hetero ring.

The hydrocarbon ring may be an aromatic ring, an aliphatic ring, or a fused ring of the aromatic ring and the aliphatic ring, and may be selected from the examples of the cycloalkyl group or the aryl group.

In the present specification, being bonded to an adjacent group to form a ring means being bonded to an adjacent group to form a substituted or unsubstituted aliphatic hydrocarbon ring; a substituted or unsubstituted aromatic hydrocarbon ring; a substituted or unsubstituted aliphatic hetero ring; a substituted or unsubstituted aromatic hetero ring; or a fused ring thereof. The hydrocarbon ring means a ring composed only of carbon and hydrogen atoms. The hetero ring means a ring including one or more selected from among N, O, P, S, Si and Se. In the present specification, the aliphatic hydrocarbon ring, the aromatic hydrocarbon ring, the aliphatic hetero ring, and the aromatic hetero ring may be monocyclic or polycyclic.

In the present specification, the aliphatic hydrocarbon ring means a ring composed only of carbon and hydrogen atoms as a non-aromatic ring. Examples of the aliphatic hydrocarbon ring include cyclopropane, cyclobutane, cyclobutene, cyclopentane, cyclopentene, cyclohexane, cyclohexene, 1,4-cyclohexadiene, cycloheptane, cycloheptene, cyclooctane, cyclooctene, and the like, but are not limited thereto.

In the present specification, an aromatic hydrocarbon ring means an aromatic ring composed only of carbon and hydrogen atoms. Examples of the aromatic hydrocarbon ring include benzene, naphthalene, anthracene, phenanthrene, perylene, fluoranthene, triphenylene, phenalene, pyrene, tetracene, chrysene, pentacene, fluorene, indene, acenaphthylene, benzofluorene, spirofluorene, and the like, but are not limited thereto. In the present specification, the aromatic hydrocarbon ring may be interpreted to have the same meaning as the aryl group.

In the present specification, an aliphatic hetero ring means an aliphatic ring including one or more of hetero atoms. Examples of the aliphatic hetero ring include oxirane, tetrahydrofuran, 1,4-dioxane, pyrrolidine, piperidine, morpholine, oxepane, azocane, thiocane, and the like, but are not limited thereto.

In the present specification, an aromatic hetero ring means an aromatic ring including one or more of hetero atoms. Examples of the aromatic hetero ring include pyridine, pyrrole, pyrimidine, pyridazine, furan, thiophene, imidazole, pyrazole, oxazole, isoxazole, thiazole, isothiazole, triazole, oxadiazole, thiadiazole, dithiazole, tetrazole, pyran, thiopyran, diazine, oxazine, thiazine, dioxine, triazine, tetrazine, isoquinoline, quinoline, quinone, quinazoline, quinoxaline, naphthyridine, acridine, phenanthridine, diaza naphthalene, triazaindene, indole, indolizine, benzothiazole, benzoxazole, benzoimidazole, benzothiophene, benzofuran, dibenzothiophene, dibenzofuran, carbazole, benzocarbazole, dibenzocarbazole, phenazine, imidazopyridine, phenoxazine, indolocarbazole, indenocarbazole, and the like, but are not limited thereto.

In the present specification, a fused ring means a cyclic structure in a form where two or more rings share two or more atoms. The fused ring may be an aromatic hydrocarbon ring, an aliphatic hydrocarbon ring, or a fused ring of an aromatic hydrocarbon ring and an aliphatic hydrocarbon ring, but is not limited thereto.

In the present specification, the fused aromatic hydrocarbon ring group means a ring in which two or more aromatic hydrocarbon rings are fused together. Examples of the fused aromatic hydrocarbon ring group may include a naphthyl group, an anthracenyl group, a phenanthrenyl group, a pyrenyl group, a perylenyl group, a triphenylene group, a chrysenyl group, a fluorenyl group, a triphenylenyl group, and the like, but are not limited thereto.

Unless otherwise defined in the present specification, all technical and scientific terms used in the present specification have the same meaning as commonly understood by one with ordinary skill in the art to which the present invention pertains. Although methods and materials similar to or equivalent to those described in the present specification may be used in the practice or in the test of exemplary embodiments of the present invention, suitable methods and materials will be described below. All publications, patent applications, patents, and other references mentioned in the present specification are hereby incorporated by reference in their entireties, and in the case of conflict, the present specification, including definitions, will control unless a particular passage is mentioned. Further, the materials, methods, and examples are illustrative only and are not intended to be limiting.

In the present specification, the position which may be substituted with hydrogen in Chemical Formula 1 may be substituted with deuterium.

In the present specification, L1 of Chemical Formula 1 may be a substituted or unsubstituted arylene group having 6 to 60 carbon atoms, and the position which may be substituted with hydrogen in the arylene group may be substituted with deuterium.

In the present specification, Ar1 of Chemical Formula 1 may be an aryl group having 6 to 60 carbon atoms, in which one or more cyano groups are substituted, and the position which may be substituted with hydrogen in the aryl group may be substituted with deuterium.

In the present specification, Ar21 to Ar23 of Chemical Formula 1 are the same or different, and may be each independently a substituted or unsubstituted aryl group having 6 to 60 carbon atoms, and the position which may be substituted with hydrogen in the aryl group may be substituted with deuterium.

In the present specification, HAr1 of Chemical Formula 1 may be a divalent triazinyl group that is unsubstituted or substituted with one substituent selected from the group consisting of deuterium, a substituted or unsubstituted aryl group having 6 to 60 carbon atoms, and a substituted or unsubstituted heteroaryl group having 2 to 60 carbon atoms; a divalent pyrimidinyl group that is unsubstituted or substituted with one or two substituents each independently selected from the group consisting of deuterium, a substituted or unsubstituted alkyl group having 1 to 60 carbon atoms, a substituted or unsubstituted aryl group having 6 to 60 carbon atoms, and a substituted or unsubstituted heteroaryl group having 2 to 60 carbon atoms; a divalent quinazolinyl group that is unsubstituted or substituted with deuterium; a divalent benzothienopyrimidinyl group that is unsubstituted or substituted with one substituent selected from the group consisting of deuterium, a substituted or unsubstituted aryl group having 6 to 60 carbon atoms, and a substituted or unsubstituted heteroaryl group having 2 to 60 carbon atoms; or a divalent benzofuropyrimidinyl group that is unsubstituted or substituted with one substituent selected from the group consisting of deuterium, a substituted or unsubstituted aryl group having 6 to 60 carbon atoms, and a substituted or unsubstituted heteroaryl group having 2 to 60 carbon atoms, and the position which may be substituted with hydrogen in the divalent triazinyl group; the divalent pyrimidinyl group; the divalent quinazolinyl group; the divalent benzothienopyrimidinyl group; or the divalent benzofuropyrimidinyl group may be substituted with deuterium.

In the present specification, HAr2 of Chemical Formula 1 may be a triazinyl group that is unsubstituted or substituted with two substituents each independently selected from the group consisting of deuterium, a substituted or unsubstituted aryl group having 6 to 60 carbon atoms, and a substituted or unsubstituted heteroaryl group having 2 to 60 carbon atoms; a pyrimidinyl group that is unsubstituted or substituted with one to three substituents each independently selected from the group consisting of deuterium, a substituted or unsubstituted alkyl group having 1 to 60 carbon atoms, a substituted or unsubstituted aryl group having 6 to 60 carbon atoms, and a substituted or unsubstituted heteroaryl group having 2 to 60 carbon atoms; a quinazolinyl group that is unsubstituted or substituted with one substituent selected from the group consisting of deuterium, a substituted or unsubstituted aryl group having 6 to 60 carbon atoms, and a substituted or unsubstituted heteroaryl group having 2 to 60 carbon atoms; a benzothienopyrimidinyl group that is unsubstituted or substituted with one substituent selected from the group consisting of deuterium, a substituted or unsubstituted aryl group having 6 to 60 carbon atoms, and a substituted or unsubstituted heteroaryl group having 2 to 60 carbon atoms; or a benzofuropyrimidinyl group that is unsubstituted or substituted with one substituent selected from the group consisting of deuterium, a substituted or unsubstituted aryl group having 6 to 60 carbon atoms, and a substituted or unsubstituted heteroaryl group having 2 to 60 carbon atoms, and the position which may be substituted with hydrogen in the triazinyl group; the pyrimidinyl group; the quinazolinyl group; the benzothienopyrimidinyl group; or the benzofuropyrimidinyl group may be substituted with deuterium.

In the present specification, Chemical Formula 1 is any one of the following Chemical Formulae 1-1 to 1-5.

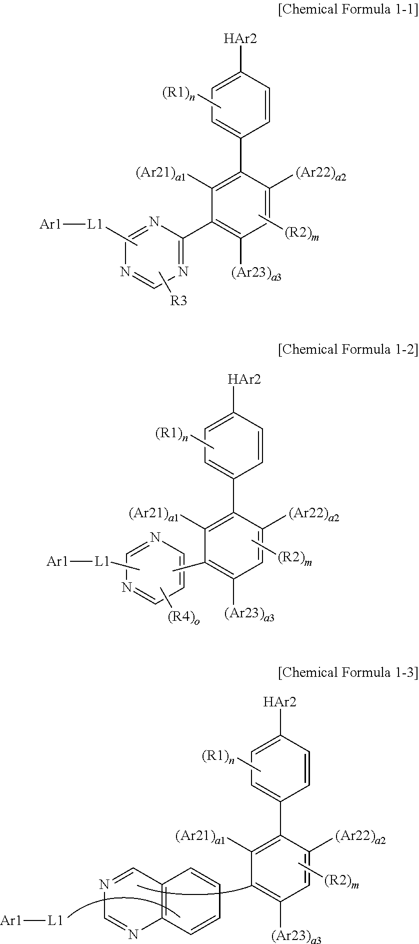

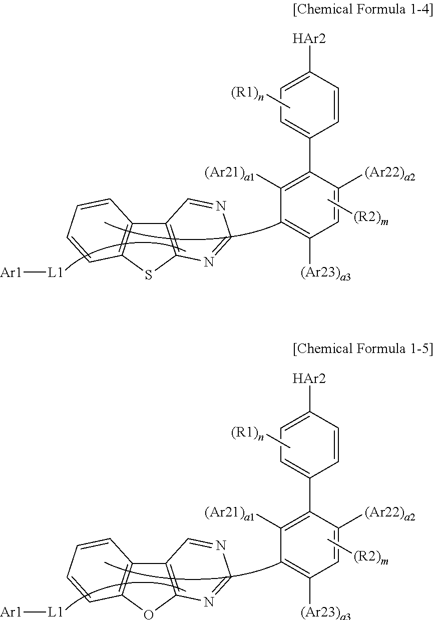

In Chemical Formulae 1-1 to 1-5,
HAr2, L1, Ar1, Ar21 to Ar23, n, m, and a1 to a3 are the same as those defined in Chemical Formula 1,
R1 and R2 are the same as or different from each other, and are each independently hydrogen or deuterium,
R3 is deuterium; a substituted or unsubstituted aryl group having 6 to 60 carbon atoms; or a substituted or unsubstituted heteroaryl group having 2 to 60 carbon atoms,
R4 is hydrogen; deuterium; a substituted or unsubstituted alkyl group having 1 to 60 carbon atoms; a substituted or unsubstituted aryl group having 6 to 60 carbon atoms; or a substituted or unsubstituted heteroaryl group having 2 to 60 carbon atoms,
o is 1 or 2, and
when o is 2, R4's are the same as or different from each other.

In the present specification, Chemical Formula 1 above is Chemical Formula 1-1 above.

In the present specification, Chemical Formula 1 above is Chemical Formula 1-2 above.

In the present specification, Chemical Formula 1 above is Chemical Formula 1-3 above.

In the present specification, Chemical Formula 1 above is Chemical Formula 1-4 above.

In the present specification, Chemical Formula 1 above is Chemical Formula 1-5 above.

According to an exemplary embodiment of the present specification, HAr1 is a divalent triazinyl group that is unsubstituted or substituted with one substituent selected from the group consisting of deuterium, a substituted or unsubstituted aryl group having 6 to 60 carbon atoms, and a substituted or unsubstituted heteroaryl group having 2 to 60 carbon atoms; a divalent pyrimidinyl group that is unsubstituted or substituted with one or two substituents each independently selected from the group consisting of deuterium, a substituted or unsubstituted alkyl group having 1 to 60 carbon atoms, a substituted or unsubstituted aryl group having 6 to 60 carbon atoms, and a substituted or unsubstituted heteroaryl group having 2 to 60 carbon atoms; a divalent quinazolinyl group that is unsubstituted or substituted with deuterium; a divalent benzothienopyrimidinyl group that is unsubstituted or substituted with one substituent selected from the group consisting of deuterium, a substituted or unsubstituted aryl group having 6 to 60 carbon atoms, and a substituted or unsubstituted heteroaryl group having 2 to 60 carbon atoms; or a divalent benzofuropyrimidinyl group that is unsubstituted or substituted with one substituent selected from the group consisting of deuterium, a substituted or unsubstituted aryl group having 6 to 60 carbon atoms, and a substituted or unsubstituted heteroaryl group having 2 to 60 carbon atoms.

According to an exemplary embodiment of the present specification, HAr1 is a divalent triazinyl group that is unsubstituted or substituted with one substituent selected from the group consisting of deuterium, a substituted or unsubstituted aryl group having 6 to 60 carbon atoms, and a substituted or unsubstituted heteroaryl group having 2 to 60 carbon atoms; a divalent pyrimidinyl group that is unsubstituted or substituted with one or two substituents each independently selected from the group consisting of deuterium, a substituted or unsubstituted alkyl group having 1 to 60 carbon atoms, a substituted or unsubstituted aryl group having 6 to 60 carbon atoms, and a substituted or unsubstituted heteroaryl group having 2 to 60 carbon atoms; a divalent quinazolinyl group that is unsubstituted or substituted with deuterium; a divalent benzothienopyrimidinyl group that is unsubstituted or substituted with deuterium; or a divalent benzofuropyrimidinyl group that is unsubstituted or substituted with deuterium.

According to an exemplary embodiment of the present specification, HAr1 is a divalent triazinyl group that is unsubstituted or substituted with one substituent selected from the group consisting of deuterium, a substituted or unsubstituted aryl group having 6 to 30 carbon atoms, and a substituted or unsubstituted heteroaryl group having 2 to 30 carbon atoms; a divalent pyrimidinyl group that is unsubstituted or substituted with one or two substituents each independently selected from the group consisting of deuterium, a substituted or unsubstituted alkyl group having 1 to 30 carbon atoms, a substituted or unsubstituted aryl group having 6 to 30 carbon atoms, and a substituted or unsubstituted heteroaryl group having 2 to 30 carbon atoms; a divalent quinazolinyl group that is unsubstituted or substituted with deuterium; a divalent benzothienopyrimidinyl group that is unsubstituted or substituted with one substituent selected from the group consisting of deuterium, a substituted or unsubstituted aryl group having 6 to 30 carbon atoms, and a substituted or unsubstituted heteroaryl group having 2 to 30 carbon atoms; or a divalent benzofuropyrimidinyl group that is unsubstituted or substituted with one substituent selected from the group consisting of deuterium, a substituted or unsubstituted aryl group having 6 to 30 carbon atoms, and a substituted or unsubstituted heteroaryl group having 2 to 30 carbon atoms.

According to an exemplary embodiment of the present specification, HAr1 is a divalent triazinyl group that is unsubstituted or substituted with one substituent selected from the group consisting of deuterium, a substituted or unsubstituted aryl group having 6 to 20 carbon atoms, and a substituted or unsubstituted heteroaryl group having 2 to 20 carbon atoms; a divalent pyrimidinyl group that is unsubstituted or substituted with one or two substituents each independently selected from the group consisting of deuterium, a substituted or unsubstituted alkyl group having 1 to 20 carbon atoms, a substituted or unsubstituted aryl group having 6 to 20 carbon atoms, and a substituted or unsubstituted heteroaryl group having 2 to 20 carbon atoms; a divalent quinazolinyl group that is unsubstituted or substituted with deuterium; a divalent benzothienopyrimidinyl group that is unsubstituted or substituted with one substituent selected from the group consisting of deuterium, a substituted or unsubstituted aryl group having 6 to 20 carbon atoms, and a substituted or unsubstituted heteroaryl group having 2 to 20 carbon atoms; or a divalent benzofuropyrimidinyl group that is unsubstituted or substituted with one substituent selected from the group consisting of deuterium, a substituted or unsubstituted aryl group having 6 to 20 carbon atoms, and a substituted or unsubstituted heteroaryl group having 2 to 20 carbon atoms.

According to an exemplary embodiment of the present specification, HAr1 is a divalent triazinyl group that is unsubstituted or substituted with one substituent selected from the group consisting of deuterium, a substituted or unsubstituted aryl group having 6 to 10 carbon atoms, and a substituted or unsubstituted heteroaryl group having 2 to 10 carbon atoms; a divalent pyrimidinyl group that is unsubstituted or substituted with one or two substituents each independently selected from the group consisting of deuterium, a substituted or unsubstituted alkyl group having 1 to 10 carbon atoms, a substituted or unsubstituted aryl group having 6 to 10 carbon atoms, and a substituted or unsubstituted heteroaryl group having 2 to 10 carbon atoms; a divalent quinazolinyl group that is unsubstituted or substituted with deuterium; a divalent benzothienopyrimidinyl group that is unsubstituted or substituted with one substituent selected from the group consisting of deuterium, a substituted or unsubstituted aryl group having 6 to 10 carbon atoms, and a substituted or unsubstituted heteroaryl group having 2 to 10 carbon atoms; or a divalent benzofuropyrimidinyl group that is unsubstituted or substituted with one substituent selected from the group consisting of deuterium, a substituted or unsubstituted aryl group having 6 to 10 carbon atoms, and a substituted or unsubstituted heteroaryl group having 2 to 10 carbon atoms.

According to an exemplary embodiment of the present specification, HAr1 is a divalent triazinyl group that is unsubstituted or substituted with one substituent selected from the group consisting of deuterium, a substituted or unsubstituted phenyl group, a substituted or unsubstituted biphenyl group, a substituted or unsubstituted naphthyl group, and a substituted or unsubstituted pyrimidinyl group; a divalent pyrimidinyl group that is unsubstituted or substituted with one or two substituents each independently selected from the group consisting of deuterium, a substituted or unsubstituted phenyl group, a substituted or unsubstituted biphenyl group, a substituted or unsubstituted naphthyl group, a substituted or unsubstituted methyl group, a substituted or unsubstituted ethyl group, a substituted or unsubstituted propyl group, and a substituted or unsubstituted pyridinyl group; a divalent quinazolinyl group that is unsubstituted or substituted with deuterium; a divalent benzothienopyrimidinyl group that is unsubstituted or substituted with one substituent selected from the group consisting of deuterium, a substituted or unsubstituted phenyl group, a substituted or unsubstituted biphenyl group, a substituted or unsubstituted naphthyl group, and a substituted or unsubstituted pyrimidinyl group; or a divalent benzofuropyrimidinyl group that is unsubstituted or substituted with one substituent selected from the group consisting of deuterium, a substituted or unsubstituted phenyl group, a substituted or unsubstituted biphenyl group, a substituted or unsubstituted naphthyl group, and a substituted or unsubstituted pyrimidinyl group.

According to an exemplary embodiment of the present specification, HAr1 is a divalent triazinyl group that is unsubstituted or substituted with one substituent selected from the group consisting of deuterium, a phenyl group that is unsubstituted or substituted with deuterium or an alkyl group, a biphenyl group that is unsubstituted or substituted with deuterium or an alkyl group, a naphthyl group that is unsubstituted or substituted with deuterium or an alkyl group, and a pyrimidinyl group that is unsubstituted or substituted with deuterium or an alkyl group; a divalent pyrimidinyl group that is unsubstituted or substituted with one or two substituents each independently selected from the group consisting of deuterium, a phenyl group that is unsubstituted or substituted with deuterium or an alkyl group, a biphenyl group that is unsubstituted or substituted with deuterium or an alkyl group, a naphthyl group that is unsubstituted or substituted with deuterium or an alkyl group, a methyl group that is unsubstituted or substituted with deuterium or an alkyl group, an ethyl group that is unsubstituted or substituted with deuterium or an alkyl group, a propyl group that is unsubstituted or substituted with deuterium or an alkyl group, and a pyridinyl group that is unsubstituted or substituted with deuterium or an alkyl group; a divalent quinazolinyl group that is unsubstituted or substituted with deuterium; a divalent benzothienopyrimidinyl group that is unsubstituted or substituted with one substituent selected from the group consisting of deuterium, a phenyl group that is unsubstituted or substituted with deuterium or an alkyl group, a biphenyl group that is unsubstituted or substituted with deuterium or an alkyl group, a naphthyl group that is unsubstituted or substituted with deuterium or an alkyl group, and pyrimidinyl group that is unsubstituted or substituted with deuterium or an alkyl group; or a divalent benzofuropyrimidinyl group that is unsubstituted or substituted with one substituent selected from the group consisting of deuterium, a phenyl group that is unsubstituted or substituted with deuterium or an alkyl group, a biphenyl group that is unsubstituted or substituted with deuterium or an alkyl group, a naphthyl group that is unsubstituted or substituted with deuterium or an alkyl group, and a pyrimidinyl group that is unsubstituted or substituted with deuterium or an alkyl group.

According to an exemplary embodiment of the present specification, HAr1 is a divalent triazinyl group that is unsubstituted or substituted with one substituent selected from the group consisting of deuterium, a phenyl group that is unsubstituted or substituted with deuterium or an alkyl group, a biphenyl group that is unsubstituted or substituted with deuterium or an alkyl group, a naphthyl group that is unsubstituted or substituted with deuterium or an alkyl group, and a pyrimidinyl group that is unsubstituted or substituted with deuterium or an alkyl group; a divalent pyrimidinyl group that is unsubstituted or substituted with one or two substituents each independently selected from the group consisting of deuterium, a phenyl group that is unsubstituted or substituted with deuterium or an alkyl group, a biphenyl group that is unsubstituted or substituted with deuterium or an alkyl group, a naphthyl group that is unsubstituted or substituted with deuterium or an alkyl group, a methyl group that is unsubstituted or substituted with deuterium or an alkyl group, an ethyl group that is unsubstituted or substituted with deuterium or an alkyl group, a propyl group that is unsubstituted or substituted with deuterium or an alkyl group, and a pyridinyl group that is unsubstituted or substituted with deuterium or an alkyl group; a divalent quinazolinyl group that is unsubstituted or substituted with deuterium; a divalent benzothienopyrimidinyl group that is unsubstituted or substituted with deuterium; or a divalent benzofuropyrimidinyl group that is unsubstituted or substituted with deuterium.

According to an exemplary embodiment of the present specification, HAr2 is a triazinyl group that is unsubstituted or substituted with two substituents each independently selected from the group consisting of deuterium, a substituted or unsubstituted aryl group having 6 to 60 carbon atoms, and a substituted or unsubstituted heteroaryl group having 2 to 60 carbon atoms; a pyrimidinyl group that is unsubstituted or substituted with one to three substituents each independently selected from the group consisting of deuterium, a substituted or unsubstituted alkyl group having 1 to 60 carbon atoms, a substituted or unsubstituted aryl group having 6 to 60 carbon atoms, and a substituted or unsubstituted heteroaryl group having 2 to 60 carbon atoms; a quinazolinyl group that is unsubstituted or substituted with one substituent selected from the group consisting of deuterium, a substituted or unsubstituted aryl group having 6 to 60 carbon atoms, and a substituted or unsubstituted heteroaryl group having 2 to 60 carbon atoms; a benzothienopyrimidinyl group that is unsubstituted or substituted with one substituent selected from the group consisting of deuterium, a substituted or unsubstituted aryl group having 6 to 60 carbon atoms, and a substituted or unsubstituted heteroaryl group having 2 to 60 carbon atoms; or a benzofuropyrimidinyl group that is unsubstituted or substituted with one substituent selected from the group consisting of deuterium, a substituted or unsubstituted aryl group having 6 to 60 carbon atoms, and a substituted or unsubstituted heteroaryl group having 2 to 60 carbon atoms.

According to an exemplary embodiment of the present specification, HAr2 is a triazinyl group that is unsubstituted or substituted with two substituents each independently selected from the group consisting of deuterium, a substituted or unsubstituted aryl group having 6 to 30 carbon atoms, and a substituted or unsubstituted heteroaryl group having 2 to 30 carbon atoms; a pyrimidinyl group that is unsubstituted or substituted with one to three substituents each independently selected from the group consisting of deuterium, a substituted or unsubstituted alkyl group having 1 to 30 carbon atoms, a substituted or unsubstituted aryl group having 6 to 30 carbon atoms, and a substituted or unsubstituted heteroaryl group having 2 to 30 carbon atoms; a quinazolinyl group that is unsubstituted or substituted with one substituent selected from the group consisting of deuterium, a substituted or unsubstituted aryl group having 6 to 30 carbon atoms, and a substituted or unsubstituted heteroaryl group having 2 to 30 carbon atoms; a benzothienopyrimidinyl group that is unsubstituted or substituted with one substituent selected from the group consisting of deuterium, a substituted or unsubstituted aryl group having 6 to 30 carbon atoms, and a substituted or unsubstituted heteroaryl group having 2 to 30 carbon atoms; or a benzofuropyrimidinyl group that is unsubstituted or substituted with one substituent selected from the group consisting of deuterium, a substituted or unsubstituted aryl group having 6 to 30 carbon atoms, and a substituted or unsubstituted heteroaryl group having 2 to 30 carbon atoms.

According to an exemplary embodiment of the present specification, HAr2 is a triazinyl group that is unsubstituted or substituted with two substituents each independently selected from the group consisting of deuterium, a substituted or unsubstituted aryl group having 6 to 20 carbon atoms, and a substituted or unsubstituted heteroaryl group having 2 to 20 carbon atoms; a pyrimidinyl group that is unsubstituted or substituted with one to three substituents each independently selected from the group consisting of deuterium, a substituted or unsubstituted alkyl group having 1 to 20 carbon atoms, a substituted or unsubstituted aryl group having 6 to 20 carbon atoms, and a substituted or unsubstituted heteroaryl group having 2 to 20 carbon atoms; a quinazolinyl group that is unsubstituted or substituted with one substituent selected from the group consisting of deuterium, a substituted or unsubstituted aryl group having 6 to 20 carbon atoms, and a substituted or unsubstituted heteroaryl group having 2 to 20 carbon atoms; a benzothienopyrimidinyl group that is unsubstituted or substituted with one substituent selected from the group consisting of deuterium, a substituted or unsubstituted aryl group having 6 to 20 carbon atoms, and a substituted or unsubstituted heteroaryl group having 2 to 20 carbon atoms; or a benzofuropyrimidinyl group that is unsubstituted or substituted with one substituent selected from the group consisting of deuterium, a substituted or unsubstituted aryl group having 6 to 20 carbon atoms, and a substituted or unsubstituted heteroaryl group having 2 to 20 carbon atoms.

According to an exemplary embodiment of the present specification, HAr2 is a triazinyl group that is unsubstituted or substituted with two substituents each independently selected from the group consisting of deuterium, a substituted or unsubstituted aryl group having 6 to 10 carbon atoms, and a substituted or unsubstituted heteroaryl group having 2 to 10 carbon atoms; a pyrimidinyl group that is unsubstituted or substituted with one to three substituents each independently selected from the group consisting of deuterium, a substituted or unsubstituted alkyl group having 1 to 10 carbon atoms, a substituted or unsubstituted aryl group having 6 to 10 carbon atoms, and a substituted or unsubstituted heteroaryl group having 2 to 10 carbon atoms; a quinazolinyl group that is unsubstituted or substituted with one substituent selected from the group consisting of deuterium, a substituted or unsubstituted aryl group having 6 to 10 carbon atoms, and a substituted or unsubstituted heteroaryl group having 2 to 10 carbon atoms; a benzothienopyrimidinyl group that is unsubstituted or substituted with one substituent selected from the group consisting of deuterium, a substituted or unsubstituted aryl group having 6 to 10 carbon atoms, and a substituted or unsubstituted heteroaryl group having 2 to 10 carbon atoms; or a benzofuropyrimidinyl group that is unsubstituted or substituted with one substituent selected from the group consisting of deuterium, a substituted or unsubstituted aryl group having 6 to 10 carbon atoms, and a substituted or unsubstituted heteroaryl group having 2 to 10 carbon atoms.

According to an exemplary embodiment of the present specification, HAr2 is a triazinyl group that is unsubstituted or substituted with two substituents each independently selected from the group consisting of deuterium, a substituted or unsubstituted phenyl group, a substituted or unsubstituted biphenyl group, a substituted or unsubstituted naphthyl group, and a substituted or unsubstituted pyrimidinyl group; a pyrimidinyl group that is unsubstituted or substituted with one to three substituents each independently selected from the group consisting of deuterium, a substituted or unsubstituted phenyl group, a substituted or unsubstituted biphenyl group, a substituted or unsubstituted naphthyl group, a substituted or unsubstituted methyl group, a substituted or unsubstituted ethyl group, a substituted or unsubstituted propyl group, and a substituted or unsubstituted pyridinyl group; a quinazolinyl group substituted with one substituent selected from the group consisting of deuterium, a substituted or unsubstituted phenyl group, a substituted or unsubstituted biphenyl group, and a substituted or unsubstituted naphthyl group; a benzothienopyrimidinyl group that is unsubstituted or substituted with one substituent each independently selected from the group consisting of deuterium, a substituted or unsubstituted phenyl group, a substituted or unsubstituted biphenyl group, a substituted or unsubstituted naphthyl group, and a substituted or unsubstituted pyrimidinyl group; or a benzofuropyrimidinyl group that is unsubstituted or substituted with one substituent each independently selected from the group consisting of deuterium, a substituted or unsubstituted phenyl group, a substituted or unsubstituted biphenyl group, a substituted or unsubstituted naphthyl group, and a substituted or unsubstituted pyrimidinyl group.

According to an exemplary embodiment of the present specification, HAr2 is a triazinyl group that is unsubstituted or substituted with two substituents each independently selected from the group consisting of deuterium, a phenyl group that is unsubstituted or substituted with deuterium or an alkyl group, a biphenyl group that is unsubstituted or substituted with deuterium or an alkyl group, a naphthyl group that is unsubstituted or substituted with deuterium or an alkyl group, and a pyrimidinyl group that is unsubstituted or substituted with deuterium or an alkyl group; a pyrimidinyl group that is unsubstituted or substituted with one to three substituents each independently selected from the group consisting of deuterium, a phenyl group that is unsubstituted or substituted with deuterium or an alkyl group, a biphenyl group that is unsubstituted or substituted with deuterium or an alkyl group, a naphthyl group that is unsubstituted or substituted with deuterium or an alkyl group, a methyl group that is unsubstituted or substituted with deuterium or an alkyl group, an ethyl group that is unsubstituted or substituted with deuterium or an alkyl group, a propyl group that is unsubstituted or substituted with deuterium or an alkyl group, and a pyridinyl group that is unsubstituted or substituted with deuterium or an alkyl group; a quinazolinyl group that is unsubstituted or substituted with one substituent selected from the group consisting of deuterium, a phenyl group that is unsubstituted or substituted with deuterium or an alkyl group, a biphenyl group that is unsubstituted or substituted with deuterium or an alkyl group, and a naphthyl group that is unsubstituted or substituted with deuterium or an alkyl group; a benzothienopyrimidinyl group that is unsubstituted or substituted with one substituent selected from the group consisting of deuterium, a phenyl group that is unsubstituted or substituted with deuterium or an alkyl group, a biphenyl group that is unsubstituted or substituted with deuterium or an alkyl group, a naphthyl group that is unsubstituted or substituted with deuterium or an alkyl group, a methyl group that is unsubstituted or substituted with deuterium or an alkyl group, an ethyl group that is unsubstituted or substituted with deuterium or an alkyl group, a propyl group that is unsubstituted or substituted with deuterium or an alkyl group, and a pyridinyl group that is unsubstituted or substituted with deuterium or an alkyl group; or a benzofuropyrimidinyl group that is unsubstituted or substituted with one substituent selected from the group consisting of deuterium, a phenyl group that is unsubstituted or substituted with deuterium or an alkyl group, a biphenyl group that is unsubstituted or substituted with deuterium or an alkyl group, a naphthyl group that is unsubstituted or substituted with deuterium or an alkyl group, a methyl group that is unsubstituted or substituted with deuterium or an alkyl group, an ethyl group that is unsubstituted or substituted with deuterium or an alkyl group, a propyl group that is unsubstituted or substituted with deuterium or an alkyl group, and a pyridinyl group that is unsubstituted or substituted with deuterium or an alkyl group.

According to an exemplary embodiment of the present specification, L1 is a substituted or unsubstituted arylene group having 6 to 60 carbon atoms.

According to an exemplary embodiment of the present specification, L1 is a substituted or unsubstituted arylene group having 6 to 30 carbon atoms.

According to an exemplary embodiment of the present specification, L1 is a substituted or unsubstituted arylene group having 6 to 20 carbon atoms.

According to an exemplary embodiment of the present specification, L1 is a substituted or unsubstituted phenylene group; a substituted or unsubstituted divalent biphenyl group; a substituted or unsubstituted divalent terphenyl group; a substituted or unsubstituted divalent naphthyl group; a substituted or unsubstituted divalent anthracenyl group; or a substituted or unsubstituted divalent phenanthrenyl group.

According to an exemplary embodiment of the present specification, L1 is a phenylene group that is unsubstituted or substituted with deuterium or an alkyl group; a divalent biphenyl group that is unsubstituted or substituted with deuterium or an alkyl group; a divalent terphenyl group that is unsubstituted or substituted with deuterium or an alkyl group; a divalent naphthyl group that is unsubstituted or substituted with deuterium or an alkyl group; a divalent anthracenyl group that is unsubstituted or substituted with deuterium or an alkyl group; or a divalent phenanthrenyl group that is unsubstituted or substituted with deuterium or an alkyl group.

According to an exemplary embodiment of the present specification, L1 is a phenylene group that is unsubstituted or substituted with deuterium, an alkyl group or an aryl group; a divalent biphenyl group that is unsubstituted or substituted with deuterium, an alkyl group or an aryl group; a divalent terphenyl group that is unsubstituted or substituted with deuterium, an alkyl group or an aryl group; a divalent naphthyl group that is unsubstituted or substituted with deuterium, an alkyl group or an aryl group; a divalent anthracenyl group that is unsubstituted or substituted with deuterium, an alkyl group or an aryl group; or a divalent phenanthrenyl group that is unsubstituted or substituted with deuterium, an alkyl group or an aryl group.

In an exemplary embodiment of the present specification, L1 is represented by any one of the following structural formulae.

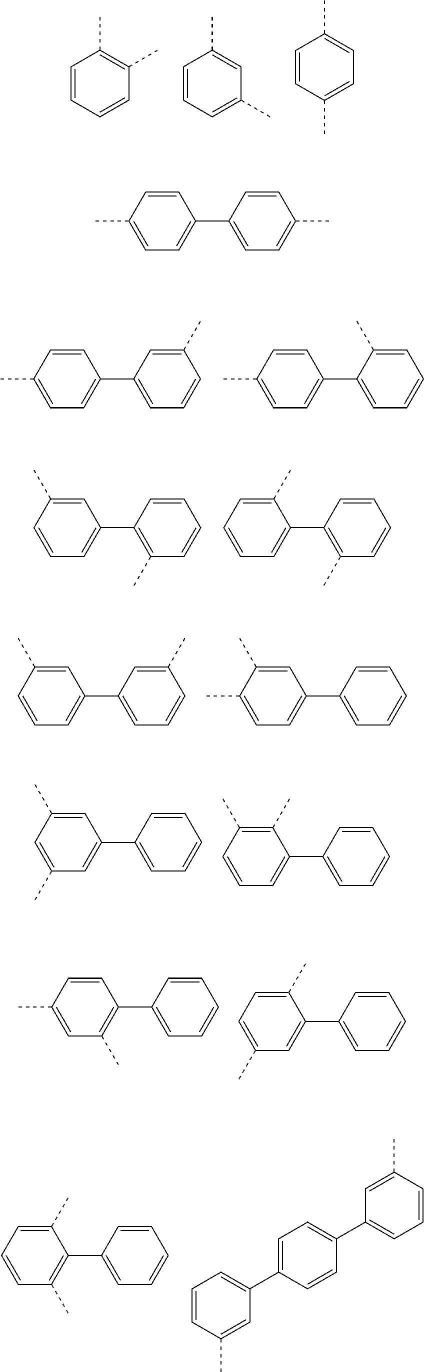

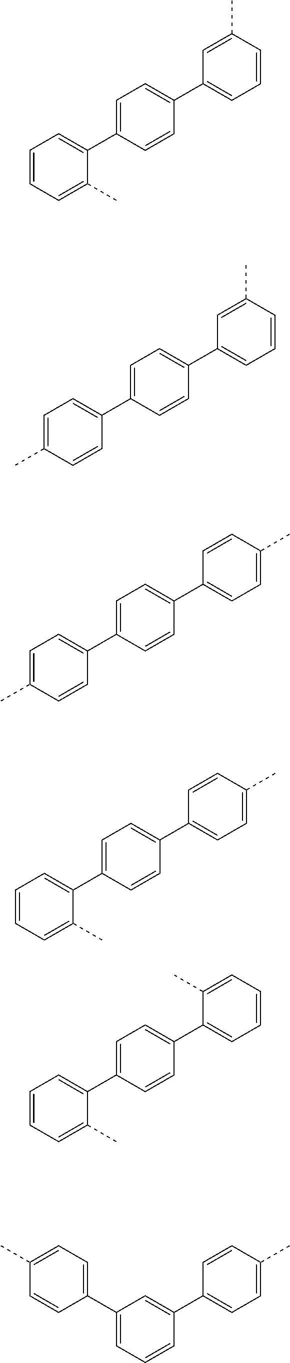

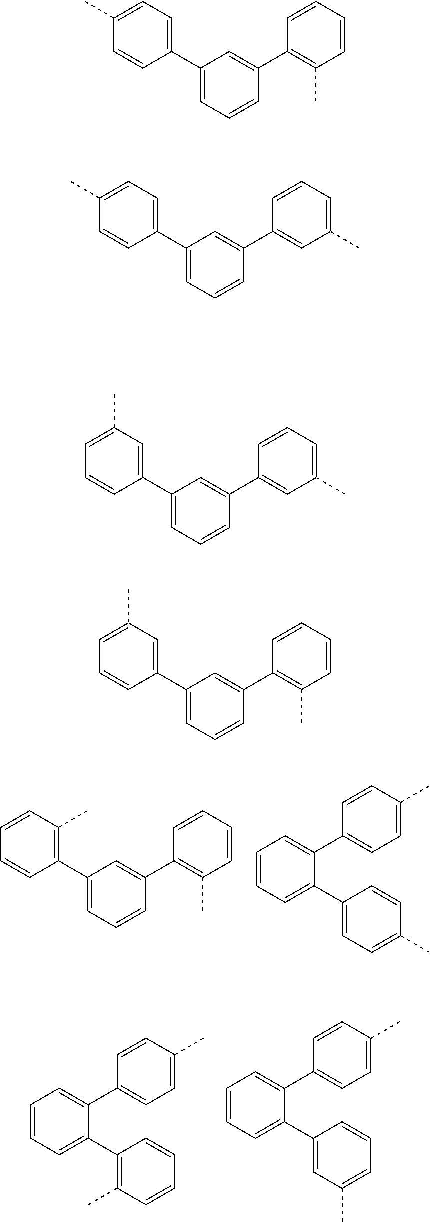

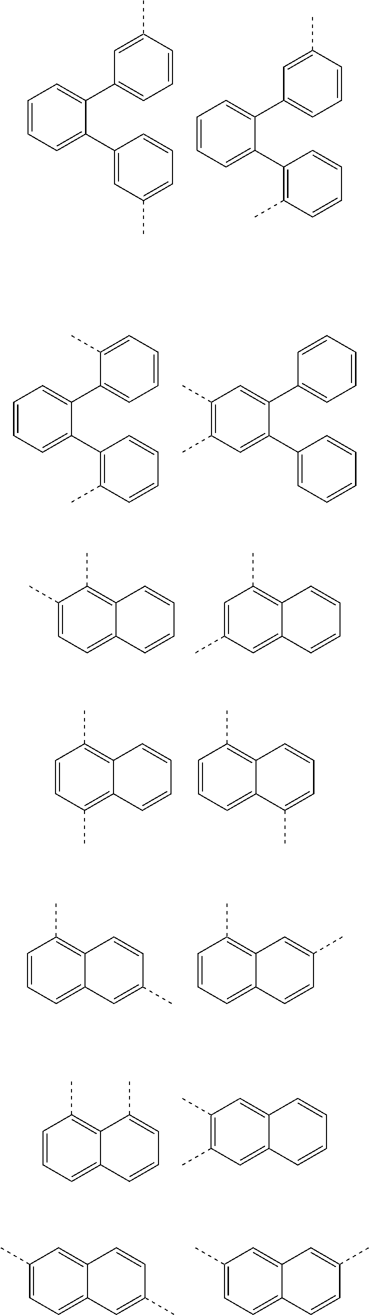

The structural formulae may be unsubstituted or substituted with deuterium, and

in the structural formulae, a dotted line means a bonding position.

According to an exemplary embodiment of the present specification, Ar1 is an aryl group having 6 to 60 carbon atoms that is substituted with one or more cyano groups, and is optionally further substituted with deuterium.

According to an exemplary embodiment of the present specification, Ar1 is an aryl group having 6 to 60 carbon atoms that is substituted with one cyano group, and is optionally further substituted with deuterium.

According to an exemplary embodiment of the present specification, Ar1 is an aryl group having 6 to 60 carbon atoms that is substituted with two cyano groups, and is optionally further substituted with deuterium.

According to an exemplary embodiment of the present specification, Ar1 is an aryl group having 6 to 60 carbon atoms that is substituted with one or two cyano groups, and is optionally further substituted with deuterium.

According to an exemplary embodiment of the present specification, Ar1 is an aryl group having 6 to 60 carbon atoms that is substituted with three cyano groups, and is optionally further substituted with deuterium.

According to an exemplary embodiment of the present specification, Ar1 is an aryl group having 6 to 30 carbon atoms that is substituted with one or more cyano groups, and is optionally further substituted with deuterium.

According to an exemplary embodiment of the present specification, Ar1 is an aryl group having 6 to 20 carbon atoms that is substituted with one or more cyano groups, and is optionally further substituted with deuterium.

According to an exemplary embodiment of the present specification, Ar1 is an aryl group having 6 to 10 carbon atoms that is substituted with one or more cyano groups, and is optionally further substituted with deuterium.

According to an exemplary embodiment of the present specification, Ar1 is a phenyl group that is substituted with one or more cyano groups, and is optionally further substituted with deuterium; a naphthyl group that is substituted with one or more cyano groups, and is optionally further substituted with deuterium; a biphenyl group that is substituted with one or more cyano groups, and is optionally further substituted with deuterium; or a terphenyl group that is substituted with one or more cyano groups, and is optionally further substituted with deuterium.

According to an exemplary embodiment of the present specification, Ar1 is represented by any one of the following structural formulae.

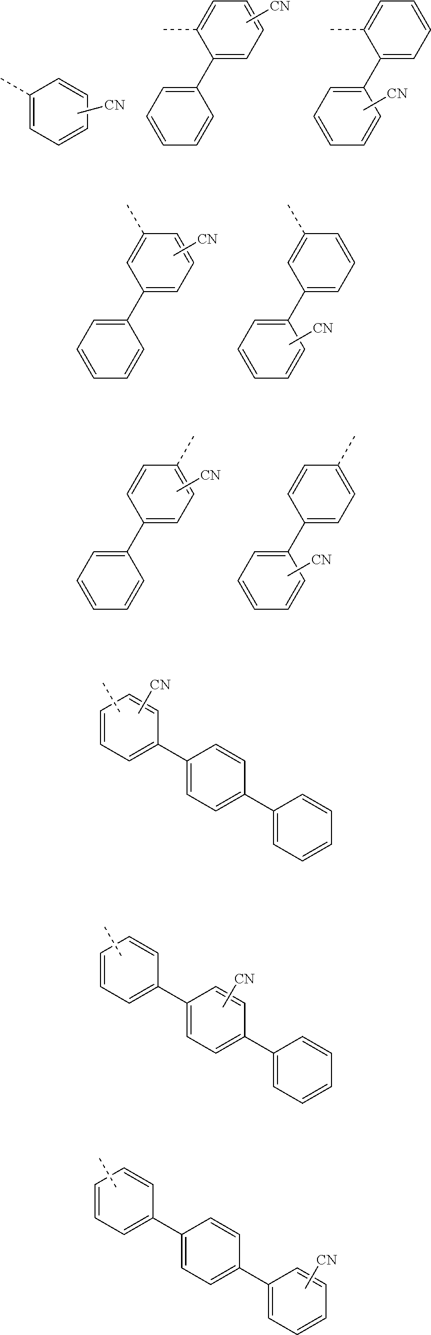

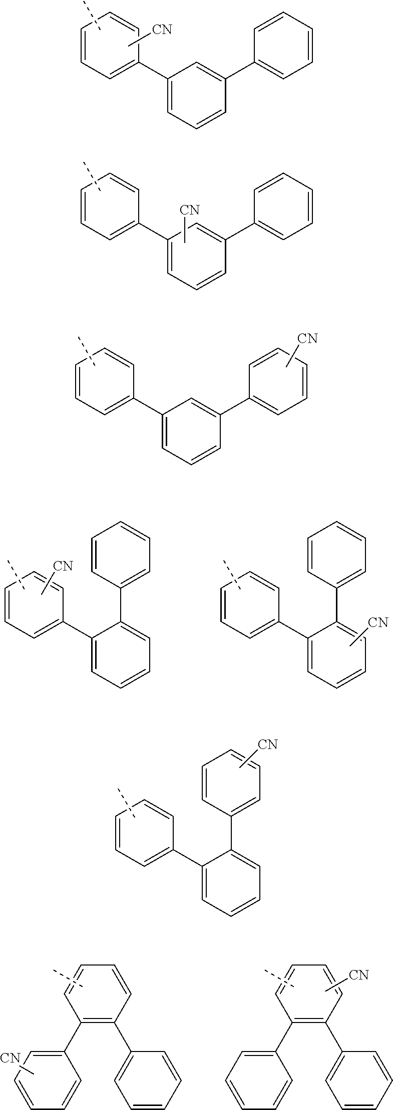

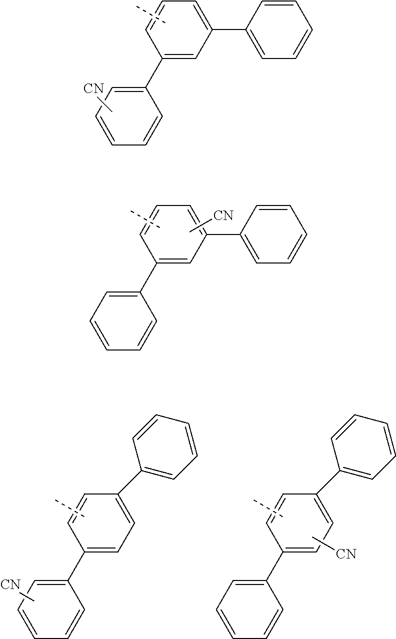

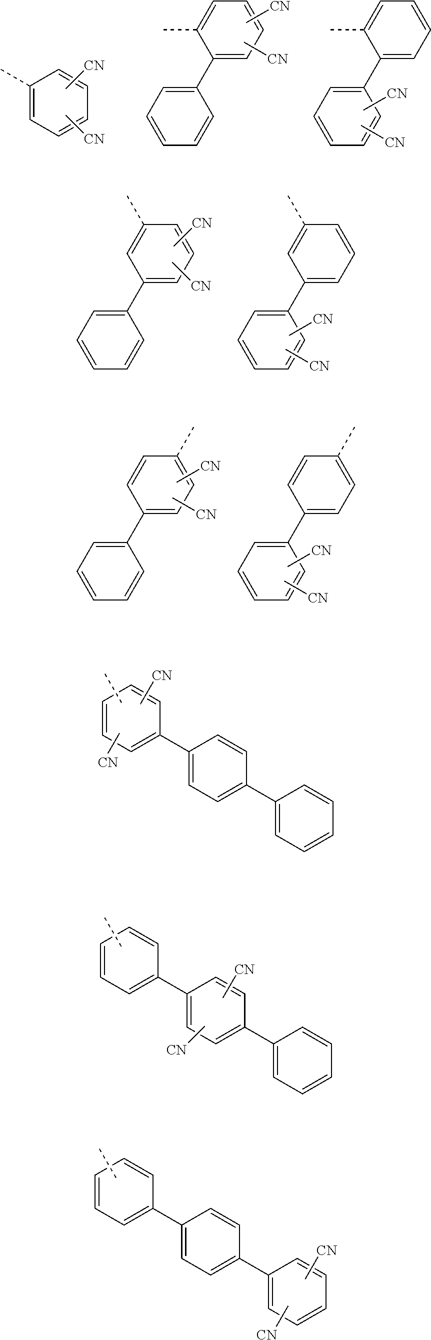

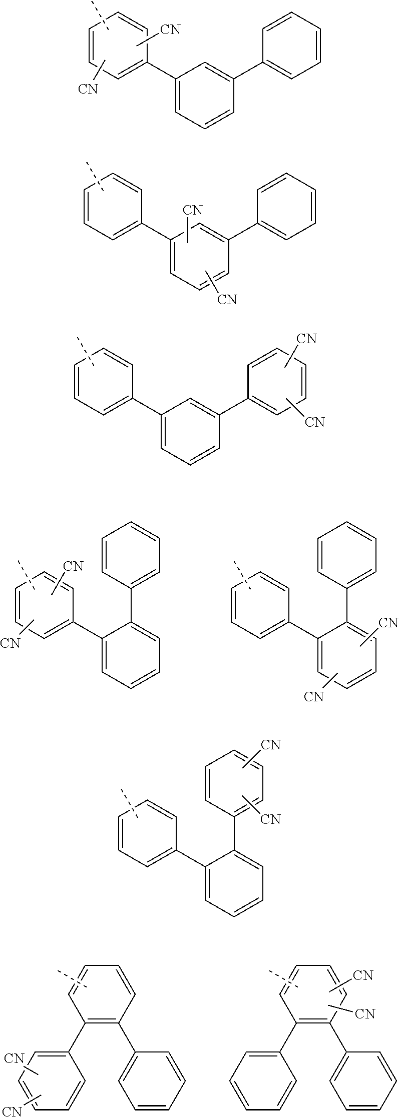

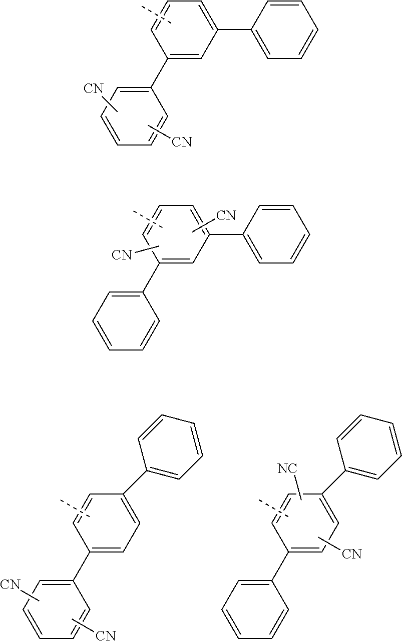

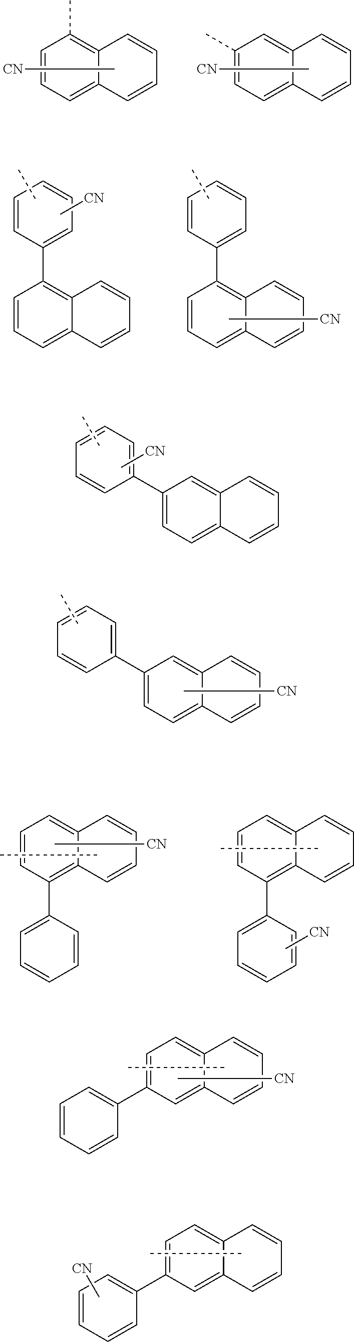

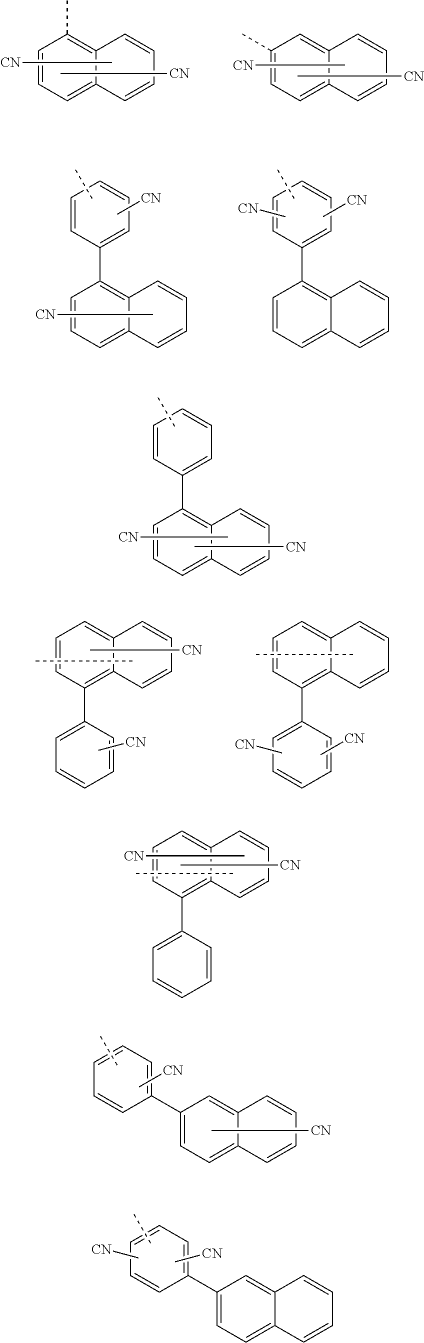

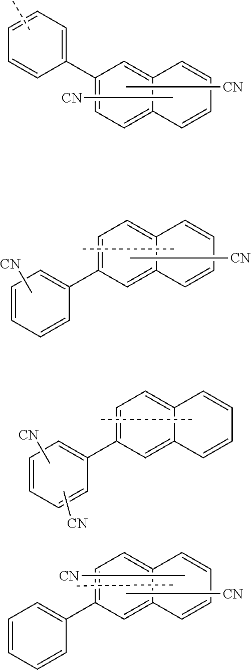

The structural formulae may be unsubstituted or substituted with deuterium, and
in the structural formulae, a dotted line means a bonding position.

According to an exemplary embodiment of the present specification, Ar21 to Ar23 are the same as or different from each other, and are each independently a substituted or unsubstituted aryl group having 6 to 60 carbon atoms.

According to an exemplary embodiment of the present specification, Ar21 to Ar23 are the same as or different from each other, and are each independently a substituted or unsubstituted aryl group having 6 to 30 carbon atoms.

According to an exemplary embodiment of the present specification, Ar21 to Ar23 are the same as or different from each other, and are each independently a substituted or unsubstituted aryl group having 6 to 20 carbon atoms.

According to an exemplary embodiment of the present specification, Ar21 to Ar23 are the same as or different from each other, and are each independently a substituted or unsubstituted phenyl group; a substituted or unsubstituted biphenyl group; a substituted or unsubstituted terphenyl group; a substituted or unsubstituted naphthyl group; a substituted or unsubstituted phenanthrenyl group; or a substituted or unsubstituted anthracenyl group.

According to an exemplary embodiment of the present specification, Ar21 to Ar23 are the same as or different from each other, and are each independently a phenyl group that is unsubstituted or substituted with deuterium or an alkyl group; a biphenyl group that is unsubstituted or substituted with deuterium or an alkyl group; a terphenyl group that is unsubstituted or substituted with deuterium or an alkyl group; a naphthyl group that is unsubstituted or substituted with deuterium or an alkyl group; a phenanthrenyl group that is unsubstituted or substituted with deuterium or an alkyl group; or an anthracenyl group that is unsubstituted or substituted with deuterium or an alkyl group.

According to an exemplary embodiment of the present specification, Ar21 to Ar23 are the same as or different from each other, and are each independently a phenyl group that is unsubstituted or substituted with deuterium, an alkyl group or an aryl group; a biphenyl group that is unsubstituted or substituted with deuterium, an alkyl group or an aryl group; a terphenyl group that is unsubstituted or substituted with deuterium, an alkyl group or an aryl group; a naphthyl group that is unsubstituted or substituted with deuterium, an alkyl group or an aryl group; a phenanthrenyl group that is unsubstituted or substituted with deuterium, an alkyl group or an aryl group; or an anthracenyl group that is unsubstituted or substituted with deuterium, an alkyl group or an aryl group.

According to an exemplary embodiment of the present specification, R1 and R2 are the same as or different from each other, and are each independently hydrogen; deuterium; or a substituted or unsubstituted alkyl group having 1 to 30 carbon atoms.

According to an exemplary embodiment of the present specification, R1 and R2 are the same as or different from each other, and are each independently hydrogen or deuterium.

According to an exemplary embodiment of the present specification, R1 and R2 are all deuterium.

According to an exemplary embodiment of the present specification, R1 and R2 are all hydrogen.

According to an exemplary embodiment of the present specification, Chemical Formula 1 is any one of the following Chemical Formulae 1-A to 1-C.

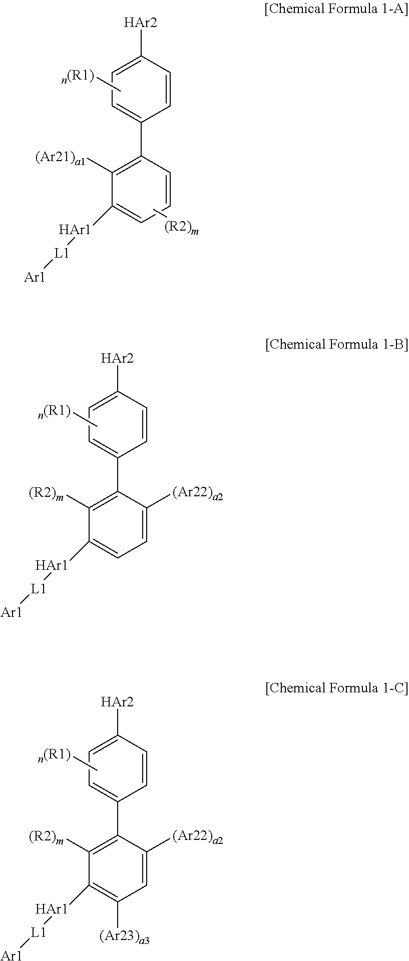

In Chemical Formulae 1-A to 1-C,

HAr1, HAr2, L1, Ar1, Ar21 to Ar23, R1, R2, n, and m are the same as those defined in Chemical formula 1, and
a1 to a3 are 1.

According to an exemplary embodiment of the present specification, Chemical Formula 1-3 is the following Chemical Formula 1-3-1 or 1-3-2.

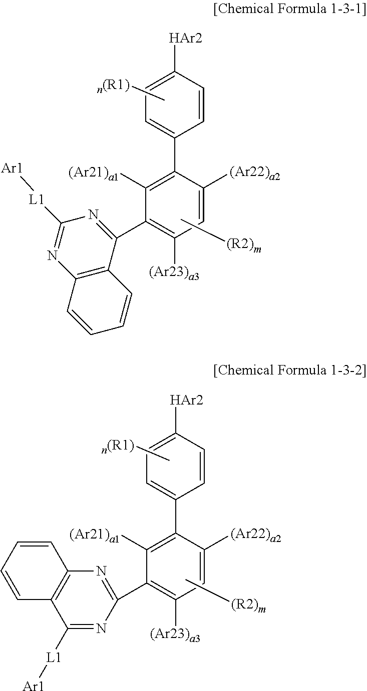

In Chemical Formulae 1-3-1 and 1-3-2,
Ar1, Ar21 to Ar23, HAr2, L1, R1, R2, a1 to a3, m, and n are the same as those defined in Chemical Formula 1-3.

According to an exemplary embodiment of the present specification, Chemical Formula 1-4 is the following Chemical Formula 1-4-1 or 1-4-2.

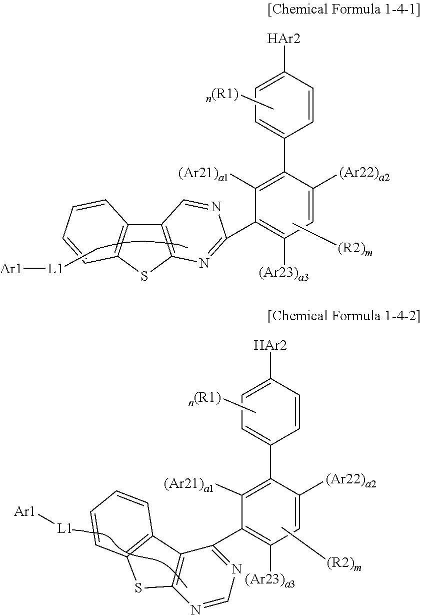

In Chemical Formulae 1-4-1 and 1-4-2,
Ar1, Ar21 to Ar23, HAr2, L1, R1, R2, a1 to a3, m, and n are the same as those defined in Chemical Formula 1-4.

According to an exemplary embodiment of the present specification, Chemical Formula 1-5 is the following Chemical Formula 1-5-1 or 1-5-2.

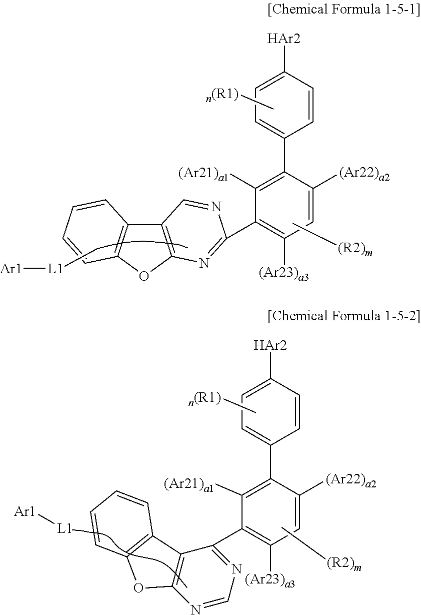

In Chemical Formulae 1-5-1 and 1-5-2,
Ar1, Ar21 to Ar23, HAr2, L1, R1, R2, a1 to a3, m, and n are the same as those defined in Chemical Formula 1-5.

In the present specification, Chemical Formula 1-3 is Chemical Formula 1-3-1 above.

In the present specification, Chemical Formula 1-3 is Chemical Formula 1-3-2 above.

In the present specification, Chemical Formula 1-4 is Chemical Formula 1-4-1 above.

In the present specification, Chemical Formula 1-4 is Chemical Formula 1-4-2 above.

In the present specification, Chemical Formula 1-5 is Chemical Formula 1-5-1 above.

In the present specification, Chemical Formula 1-5 is Chemical Formula 1-5-2 above.

According to an exemplary embodiment of the present specification, R3 is deuterium; a substituted or unsubstituted aryl group having 6 to 60 carbon atoms; or a substituted or unsubstituted heteroaryl group having 2 to 60 carbon atoms.

According to an exemplary embodiment of the present specification, R3 is deuterium; a substituted or unsubstituted aryl group having 6 to 30 carbon atoms; or a substituted or unsubstituted heteroaryl group having 2 to 30 carbon atoms.

According to an exemplary embodiment of the present specification, R3 is deuterium; a substituted or unsubstituted aryl group having 6 to 20 carbon atoms; or a substituted or unsubstituted heteroaryl group having 2 to 20 carbon atoms.

According to an exemplary embodiment of the present specification, R3 is deuterium; a substituted or unsubstituted aryl group having 6 to 10 carbon atoms; or a substituted or unsubstituted heteroaryl group having 2 to 10 carbon atoms.

According to an exemplary embodiment of the present specification, R3 is deuterium; a substituted or unsubstituted phenyl group; a substituted or unsubstituted biphenyl group; a substituted or unsubstituted naphthyl group; or a substituted or unsubstituted pyridinyl group.

According to an exemplary embodiment of the present specification, R3 is deuterium; a phenyl group that is unsubstituted or substituted with deuterium, a methyl group, or a pyrimidinyl group; a biphenyl group that is unsubstituted or substituted with deuterium, a methyl group, or a pyrimidinyl group; a naphthyl group that is unsubstituted or substituted with deuterium, a methyl group, or a pyrimidinyl group; or a pyridinyl group that is unsubstituted or substituted with deuterium, a methyl group, or a pyrimidinyl group.

According to an exemplary embodiment of the present specification, R4 is hydrogen; deuterium; a substituted or unsubstituted alkyl group having 1 to 60 carbon atoms; a substituted or unsubstituted aryl group having 6 to 60 carbon atoms; or a substituted or unsubstituted heteroaryl group having 2 to 60 carbon atoms.

According to an exemplary embodiment of the present specification, R4 is hydrogen; deuterium; a substituted or unsubstituted alkyl group having 1 to 30 carbon atoms; a substituted or unsubstituted aryl group having 6 to 30 carbon atoms; or a substituted or unsubstituted heteroaryl group having 2 to 30 carbon atoms.

According to an exemplary embodiment of the present specification, R4 is hydrogen; deuterium; a substituted or unsubstituted alkyl group having 1 to 20 carbon atoms; a substituted or unsubstituted aryl group having 6 to 20 carbon atoms; or a substituted or unsubstituted heteroaryl group having 2 to 20 carbon atoms.

According to an exemplary embodiment of the present specification, R4 is hydrogen; deuterium; a substituted or unsubstituted alkyl group having 1 to 10 carbon atoms; a substituted or unsubstituted aryl group having 6 to 10 carbon atoms; or a substituted or unsubstituted heteroaryl group having 2 to 10 carbon atoms.

According to an exemplary embodiment of the present specification, R4 is hydrogen; deuterium; a methyl group; an ethyl group; a propyl group; a substituted or unsubstituted phenyl group; a substituted or unsubstituted biphenyl group; a substituted or unsubstituted naphthyl group; or a substituted or unsubstituted pyridinyl group.

According to an exemplary embodiment of the present specification, R4 is hydrogen; deuterium; a methyl group; an ethyl group; a propyl group; a phenyl group that is unsubstituted or substituted with deuterium or an alkyl group; a biphenyl group that is unsubstituted or substituted with deuterium or an alkyl group; a naphthyl group that is unsubstituted or substituted with deuterium or an alkyl group; or a pyridinyl group that is unsubstituted or substituted with deuterium or an alkyl group.

According to an exemplary embodiment of the present specification, n is an integer from 1 to 4.

According to an exemplary embodiment of the present specification, n is 1.

According to an exemplary embodiment of the present specification, n is 2.

According to an exemplary embodiment of the present specification, n is 3.

According to an exemplary embodiment of the present specification, n is 4.

According to an exemplary embodiment of the present specification, m is an integer from 1 to 3.

According to an exemplary embodiment of the present specification, m is 1.

According to an exemplary embodiment of the present specification, m is 2.

According to an exemplary embodiment of the present specification, m is 3.

According to an exemplary embodiment of the present specification, o is 1 or 2.

According to an exemplary embodiment of the present specification, o is 1.

According to an exemplary embodiment of the present specification, o is 2.

According to an exemplary embodiment of the present specification, a1 is 0 or 1.

According to an exemplary embodiment of the present specification, a1 is 0.

According to an exemplary embodiment of the present specification, a1 is 1.

According to an exemplary embodiment of the present specification, a2 is 0 or 1.

According to an exemplary embodiment of the present specification, a2 is 0.

According to an exemplary embodiment of the present specification, a2 is 1.

According to an exemplary embodiment of the present specification, a3 is 0 or 1.

According to an exemplary embodiment of the present specification, a3 is 0.

According to an exemplary embodiment of the present specification, a3 is 1.

According to an exemplary embodiment of the present specification, Chemical Formula 1 is any one of the following structural formulae.

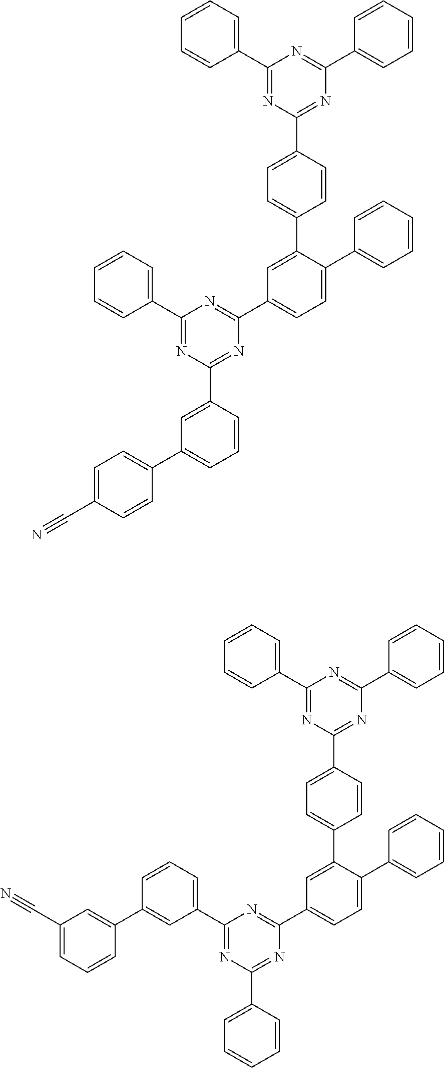 [SMILES: N#Cc1ccc(-c2cccc(-c3nc(-c4ccccc4)nc(-c4ccc(-c5ccccc5)c(-c5ccc(-c6nc(-c7ccccc7)nc(-c7ccccc7)n6)cc5)c4)n3)c2)cc1 ; N#Cc1cccc(-c2cccc(-c3nc(-c4ccccc4)nc(-c4ccc(-c5ccccc5)c(-c5ccc(-c6nc(-c7ccccc7)nc(-c7ccccc7)n6)cc5)c4)n3)c2)c1]

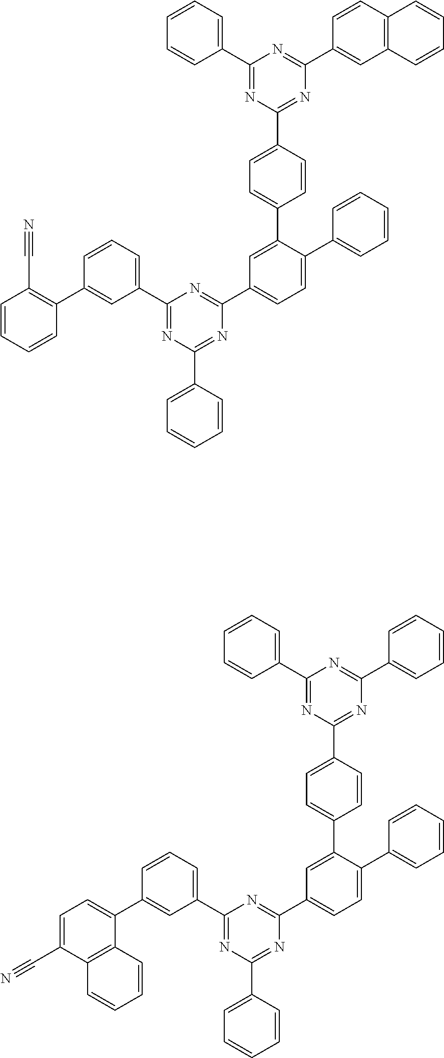 [SMILES: N#Cc1ccccc1-c1cccc(-c2nc(-c3ccccc3)nc(-c3ccc(-c4ccccc4)c(-c4ccc(-c5nc(-c6ccccc6)nc(-c6ccc7ccccc7c6)n5)cc4)c3)n2)c1 ; N#Cc1ccc(-c2cccc(-c3nc(-c4ccccc4)nc(-c4ccc(-c5ccccc5)c(-c5ccc(-c6nc(-c7ccccc7)nc(-c7ccccc7)n6)cc5)c4)n3)c2)c2ccccc12]

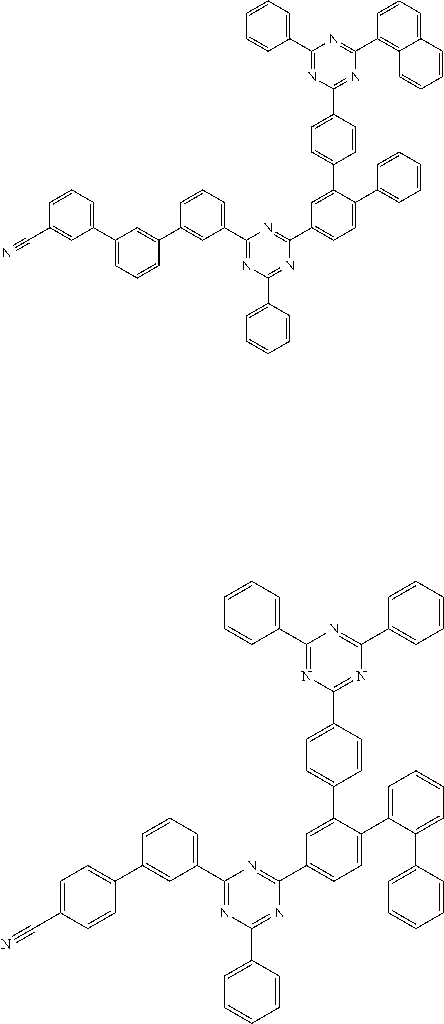 [SMILES: N#Cc1cccc(-c2cccc(-c3cccc(-c4nc(-c5ccccc5)nc(-c5ccc(-c6ccccc6)c(-c6ccc(-c7nc(-c8ccccc8)nc(-c8cccc9ccccc89)n7)cc6)c5)n4)c3)c2)c1 ; N#Cc1ccc(-c2cccc(-c3nc(-c4ccccc4)nc(-c4ccc(-c5ccccc5-c5ccccc5)c(-c5ccc(-c6nc(-c7ccccc7)nc(-c7ccccc7)n6)cc5)c4)n3)c2)cc1]

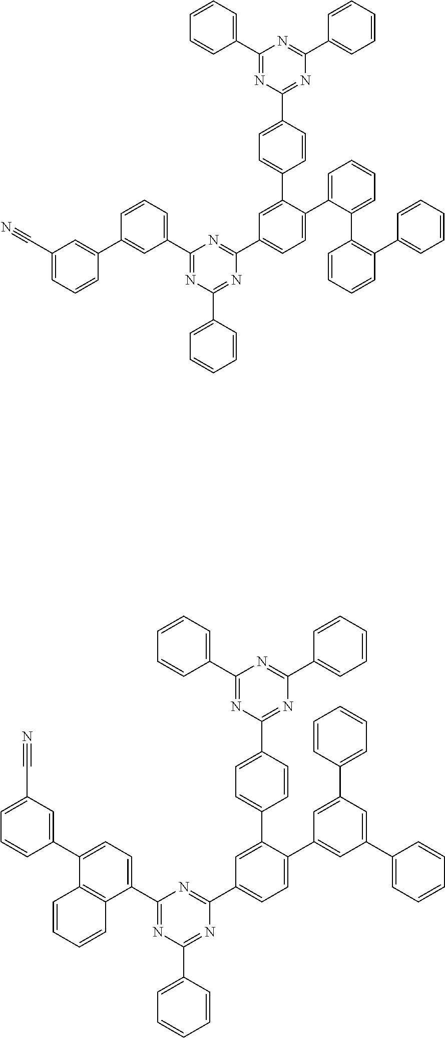 [SMILES: N#Cc1cccc(-c2cccc(-c3nc(-c4ccccc4)nc(-c4ccc(-c5ccccc5-c5ccccc5-c5ccccc5)c(-c5ccc(-c6nc(-c7ccccc7)nc(-c7ccccc7)n6)cc5)c4)n3)c2)c1 ; N#Cc1cccc(-c2ccc(-c3nc(-c4ccccc4)nc(-c4ccc(-c5cc(-c6ccccc6)cc(-c6ccccc6)c5)c(-c5ccc(-c6nc(-c7ccccc7)nc(-c7ccccc7)n6)cc5)c4)n3)c3ccccc23)c1]

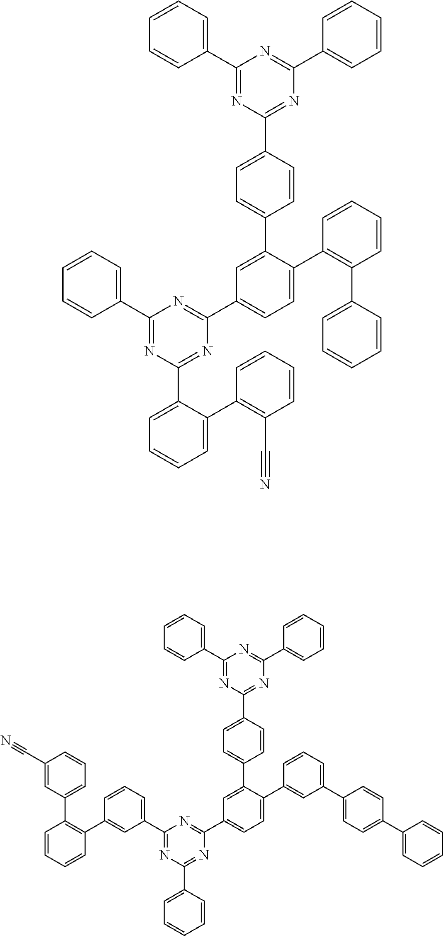 [SMILES: N#Cc1ccccc1-c1ccccc1-c1nc(-c2ccccc2)nc(-c2ccc(-c3ccccc3-c3ccccc3)c(-c3ccc(-c4nc(-c5ccccc5)nc(-c5ccccc5)n4)cc3)c2)n1 ; N#Cc1cccc(-c2ccccc2-c2cccc(-c3nc(-c4ccccc4)nc(-c4ccc(-c5cccc(-c6ccc(-c7ccccc7)cc6)c5)c(-c5ccc(-c6nc(-c7ccccc7)nc(-c7ccccc7)n6)cc5)c4)n3)c2)c1]

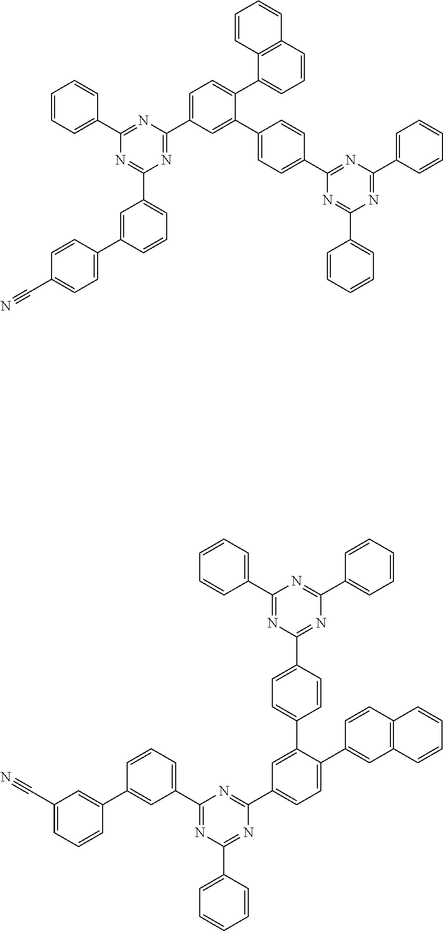 [SMILES: N#Cc1ccc(-c2cccc(-c3nc(-c4ccccc4)nc(-c4ccc(-c5cccc6ccccc56)c(-c5ccc(-c6nc(-c7ccccc7)nc(-c7ccccc7)n6)cc5)c4)n3)c2)cc1 ; N#Cc1cccc(-c2cccc(-c3nc(-c4ccccc4)nc(-c4ccc(-c5ccc6ccccc6c5)c(-c5ccc(-c6nc(-c7ccccc7)nc(-c7ccccc7)n6)cc5)c4)n3)c2)c1]

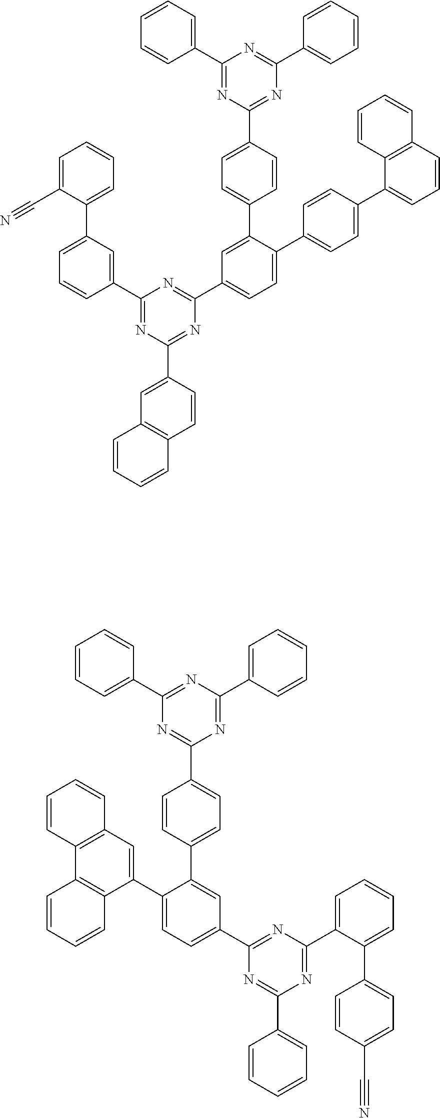 [SMILES: N#Cc1ccccc1-c1cccc(-c2nc(-c3ccc(-c4ccc(-c5cccc6ccccc56)cc4)c(-c4ccc(-c5nc(-c6ccccc6)nc(-c6ccccc6)n5)cc4)c3)nc(-c3ccc4ccccc4c3)n2)c1 ; N#Cc1ccc(-c2ccccc2-c2nc(-c3ccccc3)nc(-c3ccc(-c4cc5ccccc5c5ccccc45)c(-c4ccc(-c5nc(-c6ccccc6)nc(-c6ccccc6)n5)cc4)c3)n2)cc1]

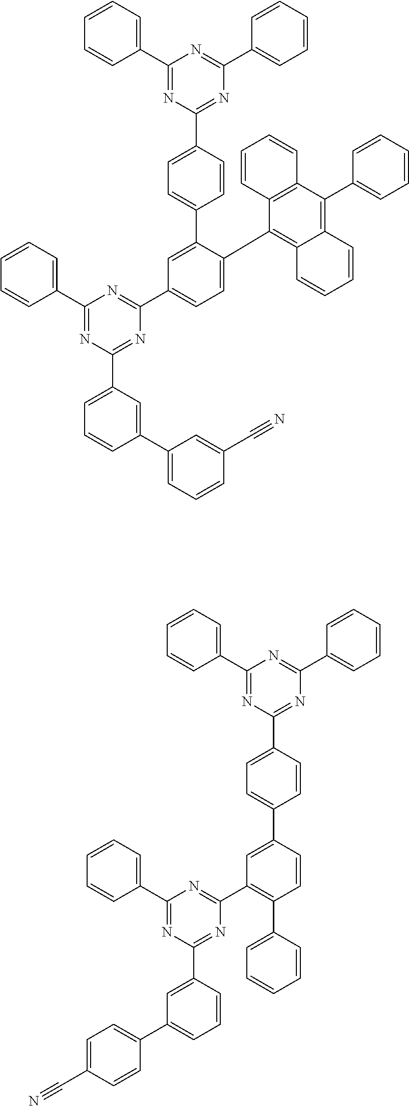 [SMILES: N#Cc1cccc(-c2cccc(-c3nc(-c4ccccc4)nc(-c4ccc(-c5c6ccccc6c(-c6ccccc6)c6ccccc56)c(-c5ccc(-c6nc(-c7ccccc7)nc(-c7ccccc7)n6)cc5)c4)n3)c2)c1 ; N#Cc1ccc(-c2cccc(-c3nc(-c4ccccc4)nc(-c4cc(-c5ccc(-c6nc(-c7ccccc7)nc(-c7ccccc7)n6)cc5)ccc4-c4ccccc4)n3)c2)cc1]

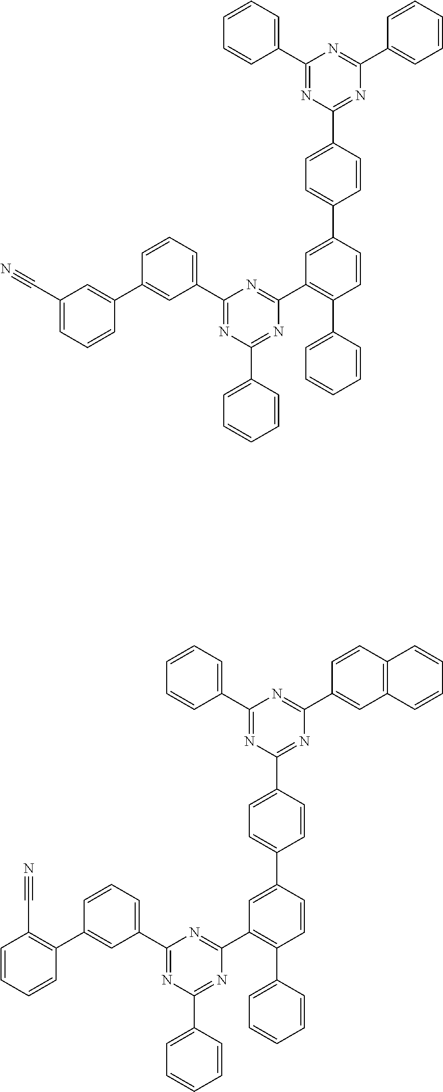 [SMILES: N#Cc1cccc(-c2cccc(-c3nc(-c4ccccc4)nc(-c4cc(-c5ccc(-c6nc(-c7ccccc7)nc(-c7ccccc7)n6)cc5)ccc4-c4ccccc4)n3)c2)c1 ; N#Cc1ccccc1-c1cccc(-c2nc(-c3ccccc3)nc(-c3cc(-c4ccc(-c5nc(-c6ccccc6)nc(-c6ccc7ccccc7c6)n5)cc4)ccc3-c3ccccc3)n2)c1]

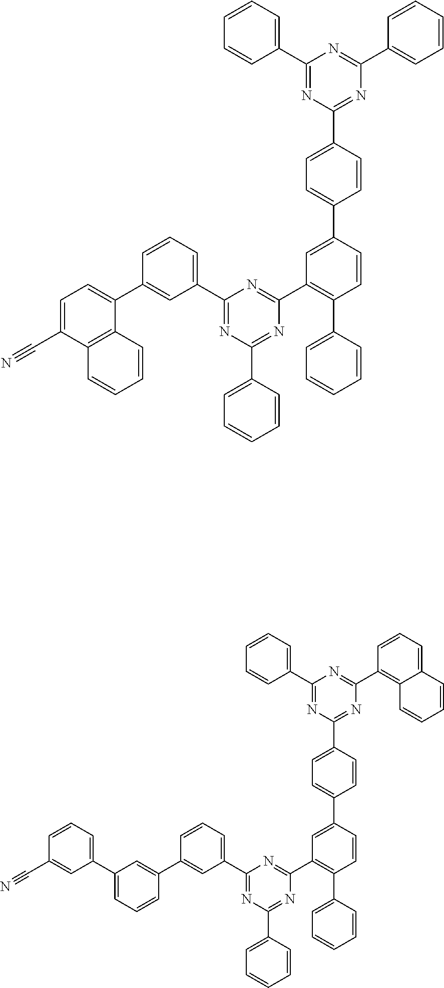 [SMILES: N#Cc1ccc(-c2cccc(-c3nc(-c4ccccc4)nc(-c4cc(-c5ccc(-c6nc(-c7ccccc7)nc(-c7ccccc7)n6)cc5)ccc4-c4ccccc4)n3)c2)c2ccccc12 ; N#Cc1cccc(-c2cccc(-c3cccc(-c4nc(-c5ccccc5)nc(-c5cc(-c6ccc(-c7nc(-c8ccccc8)nc(-c8cccc9ccccc89)n7)cc6)ccc5-c5ccccc5)n4)c3)c2)c1]

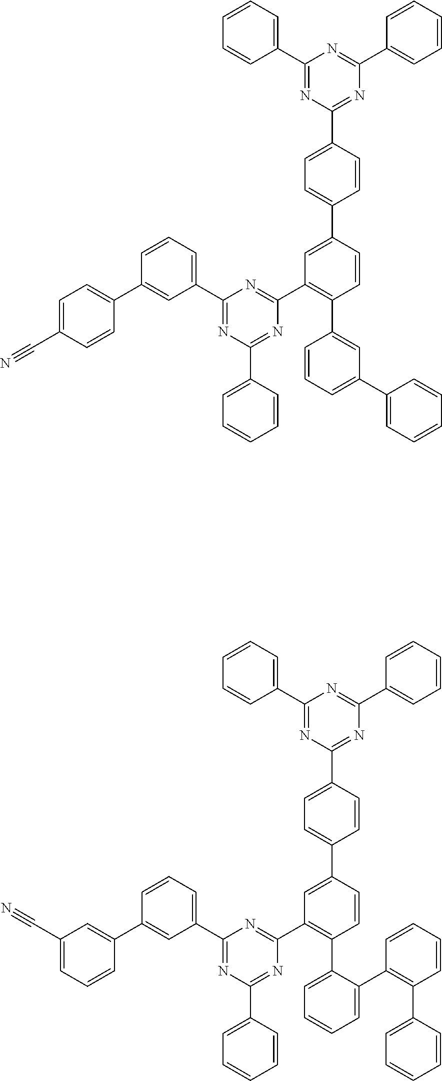 [SMILES: N#Cc1ccc(-c2cccc(-c3nc(-c4ccccc4)nc(-c4cc(-c5ccc(-c6nc(-c7ccccc7)nc(-c7ccccc7)n6)cc5)ccc4-c4cccc(-c5ccccc5)c4)n3)c2)cc1 ; N#Cc1cccc(-c2cccc(-c3nc(-c4ccccc4)nc(-c4cc(-c5ccc(-c6nc(-c7ccccc7)nc(-c7ccccc7)n6)cc5)ccc4-c4ccccc4-c4ccccc4-c4ccccc4)n3)c2)c1]

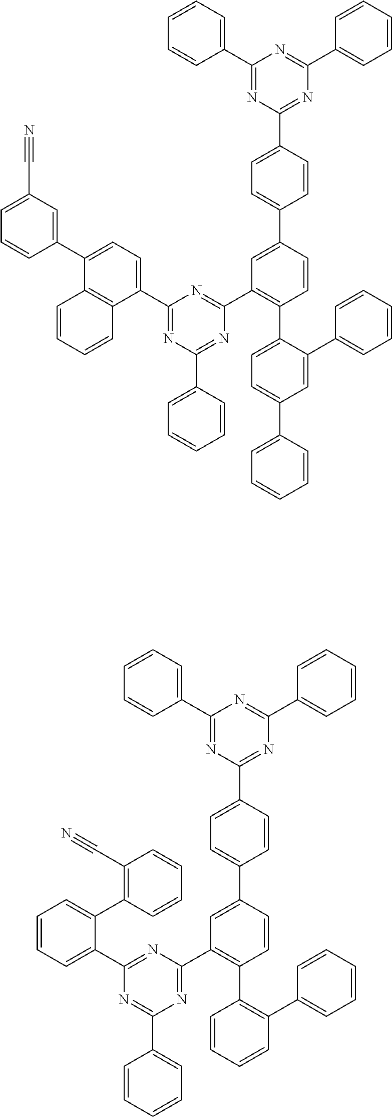 [SMILES: N#Cc1cccc(-c2ccc(-c3nc(-c4ccccc4)nc(-c4cc(-c5ccc(-c6nc(-c7ccccc7)nc(-c7ccccc7)n6)cc5)ccc4-c4ccc(-c5ccccc5)cc4-c4ccccc4)n3)c3ccccc23)c1 ; N#Cc1ccccc1-c1ccccc1-c1nc(-c2ccccc2)nc(-c2cc(-c3ccc(-c4nc(-c5ccccc5)nc(-c5ccccc5)n4)cc3)ccc2-c2ccccc2-c2ccccc2)n1]

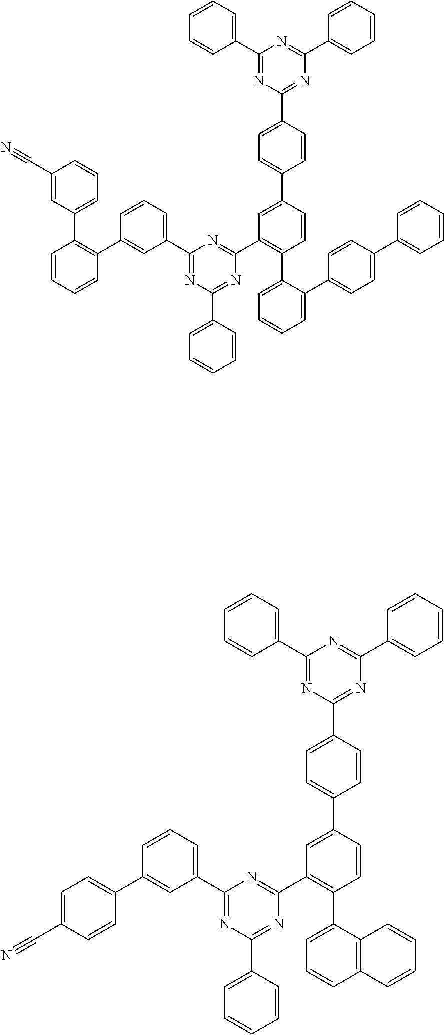 [SMILES: N#Cc1cccc(-c2ccccc2-c2cccc(-c3nc(-c4ccccc4)nc(-c4cc(-c5ccc(-c6nc(-c7ccccc7)nc(-c7ccccc7)n6)cc5)ccc4-c4ccccc4-c4ccc(-c5ccccc5)cc4)n3)c2)c1 ; N#Cc1ccc(-c2cccc(-c3nc(-c4ccccc4)nc(-c4cc(-c5ccc(-c6nc(-c7ccccc7)nc(-c7ccccc7)n6)cc5)ccc4-c4cccc5ccccc45)n3)c2)cc1]

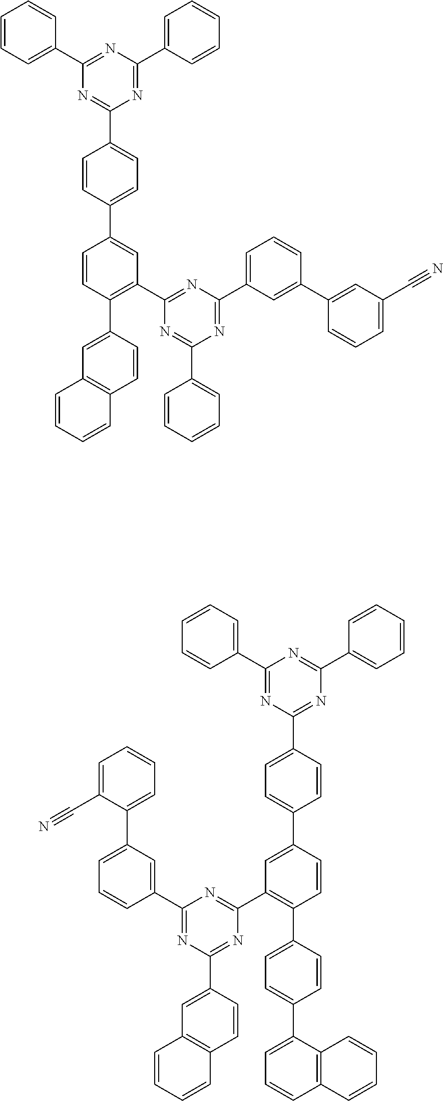 [SMILES: N#Cc1cccc(-c2cccc(-c3nc(-c4ccccc4)nc(-c4cc(-c5ccc(-c6nc(-c7ccccc7)nc(-c7ccccc7)n6)cc5)ccc4-c4ccc5ccccc5c4)n3)c2)c1 ; N#Cc1ccccc1-c1cccc(-c2nc(-c3ccc4ccccc4c3)nc(-c3cc(-c4ccc(-c5nc(-c6ccccc6)nc(-c6ccccc6)n5)cc4)ccc3-c3ccc(-c4cccc5ccccc45)cc3)n2)c1]

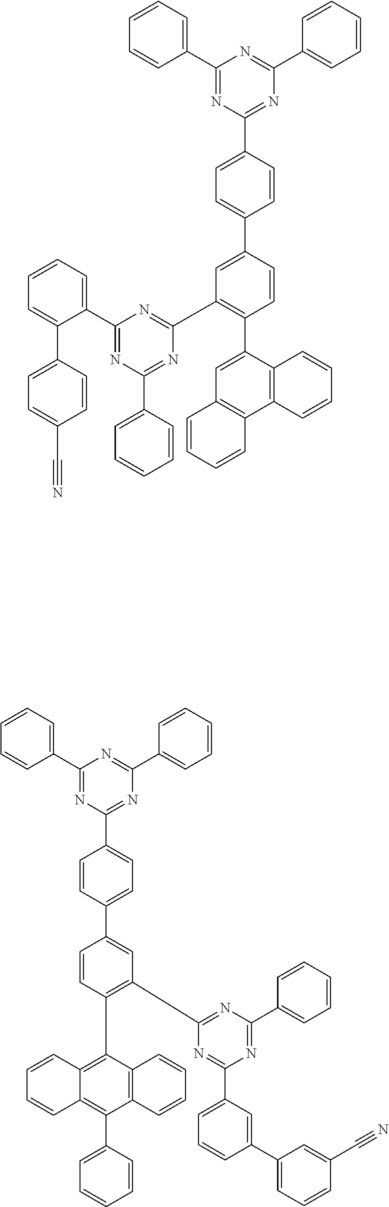 [SMILES: N#Cc1ccc(-c2ccccc2-c2nc(-c3ccccc3)nc(-c3cc(-c4ccc(-c5nc(-c6ccccc6)nc(-c6ccccc6)n5)cc4)ccc3-c3cc4ccccc4c4ccccc34)n2)cc1 ; N#Cc1cccc(-c2cccc(-c3nc(-c4ccccc4)nc(-c4cc(-c5ccc(-c6nc(-c7ccccc7)nc(-c7ccccc7)n6)cc5)ccc4-c4c5ccccc5c(-c5ccccc5)c5ccccc45)n3)c2)c1]

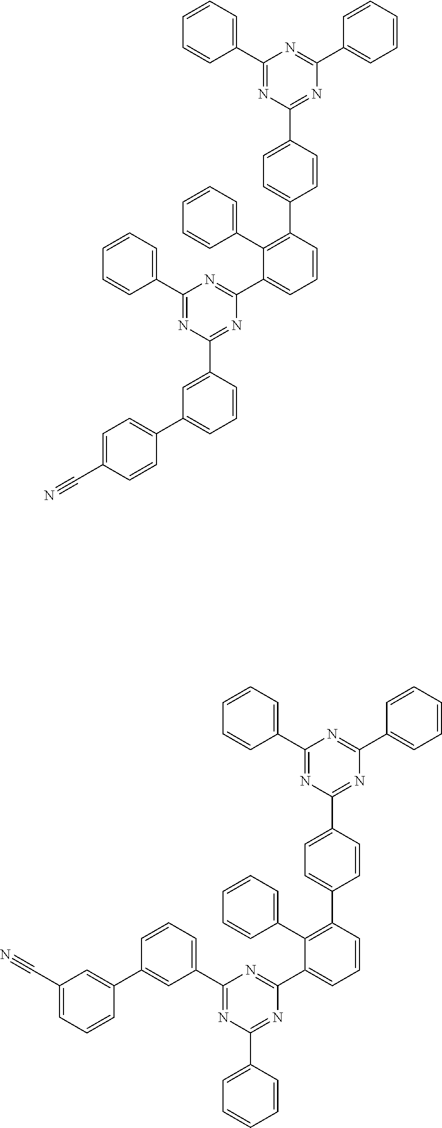 [SMILES: N#Cc1ccc(-c2cccc(-c3nc(-c4ccccc4)nc(-c4cccc(-c5ccc(-c6nc(-c7ccccc7)nc(-c7ccccc7)n6)cc5)c4-c4ccccc4)n3)c2)cc1 ; N#Cc1cccc(-c2cccc(-c3nc(-c4ccccc4)nc(-c4cccc(-c5ccc(-c6nc(-c7ccccc7)nc(-c7ccccc7)n6)cc5)c4-c4ccccc4)n3)c2)c1]

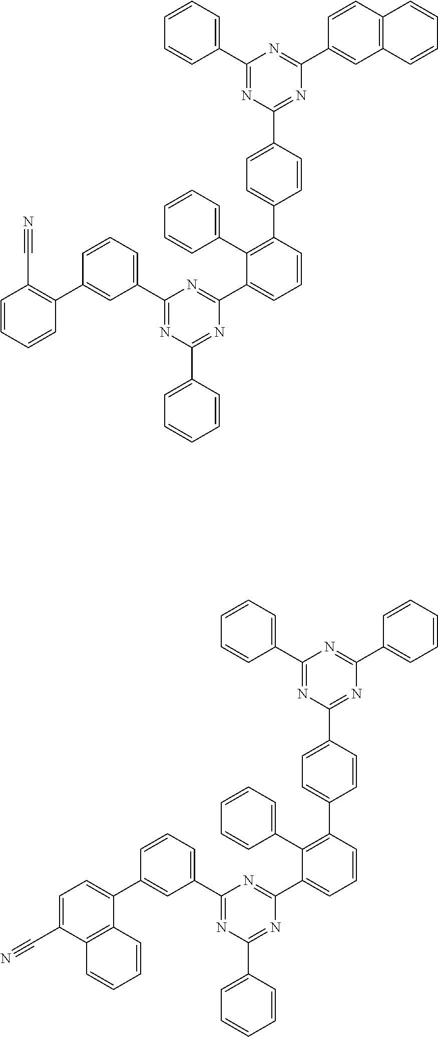 [SMILES: N#Cc1ccccc1-c1cccc(-c2nc(-c3ccccc3)nc(-c3cccc(-c4ccc(-c5nc(-c6ccccc6)nc(-c6ccc7ccccc7c6)n5)cc4)c3-c3ccccc3)n2)c1 ; N#Cc1ccc(-c2cccc(-c3nc(-c4ccccc4)nc(-c4cccc(-c5ccc(-c6nc(-c7ccccc7)nc(-c7ccccc7)n6)cc5)c4-c4ccccc4)n3)c2)c2ccccc12]

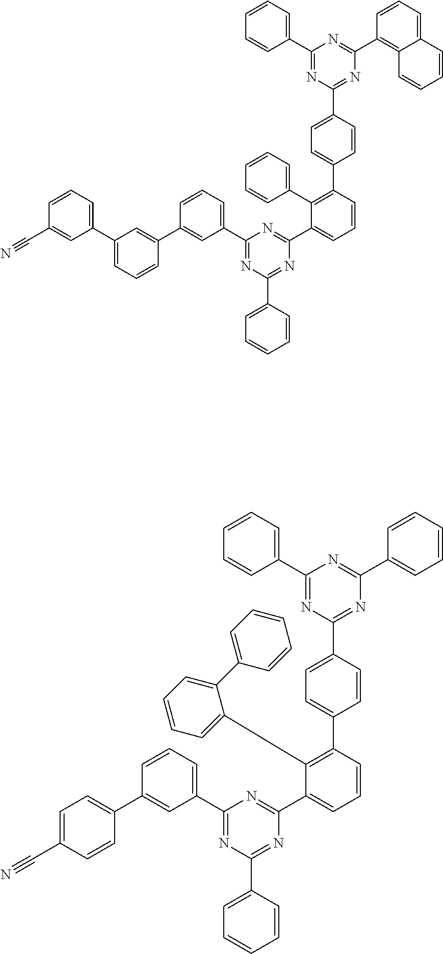 [SMILES: N#Cc1cccc(-c2cccc(-c3cccc(-c4nc(-c5ccccc5)nc(-c5cccc(-c6ccc(-c7nc(-c8ccccc8)nc(-c8cccc9ccccc89)n7)cc6)c5-c5ccccc5)n4)c3)c2)c1 ; N#Cc1ccc(-c2cccc(-c3nc(-c4ccccc4)nc(-c4cccc(-c5ccc(-c6nc(-c7ccccc7)nc(-c7ccccc7)n6)cc5)c4-c4ccccc4-c4ccccc4)n3)c2)cc1]

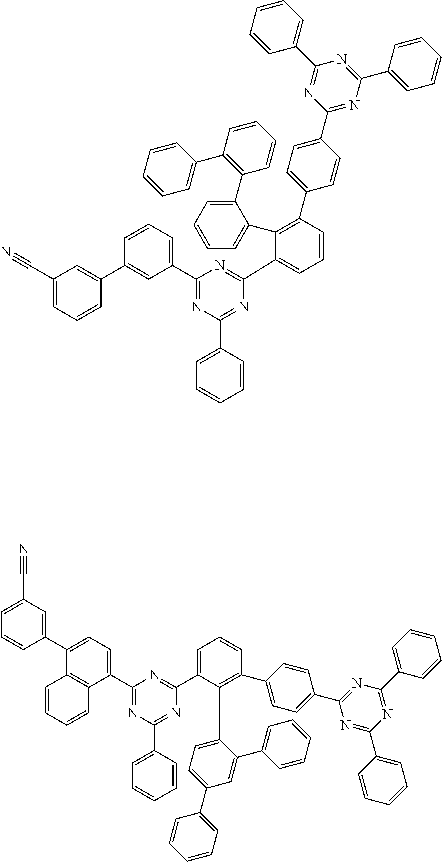 [SMILES: N#Cc1cccc(-c2cccc(-c3nc(-c4ccccc4)nc(-c4cccc(-c5ccc(-c6nc(-c7ccccc7)nc(-c7ccccc7)n6)cc5)c4-c4ccccc4-c4ccccc4-c4ccccc4)n3)c2)c1 ; N#Cc1cccc(-c2ccc(-c3nc(-c4ccccc4)nc(-c4cccc(-c5ccc(-c6nc(-c7ccccc7)nc(-c7ccccc7)n6)cc5)c4-c4ccc(-c5ccccc5)cc4-c4ccccc4)n3)c3ccccc23)c1]

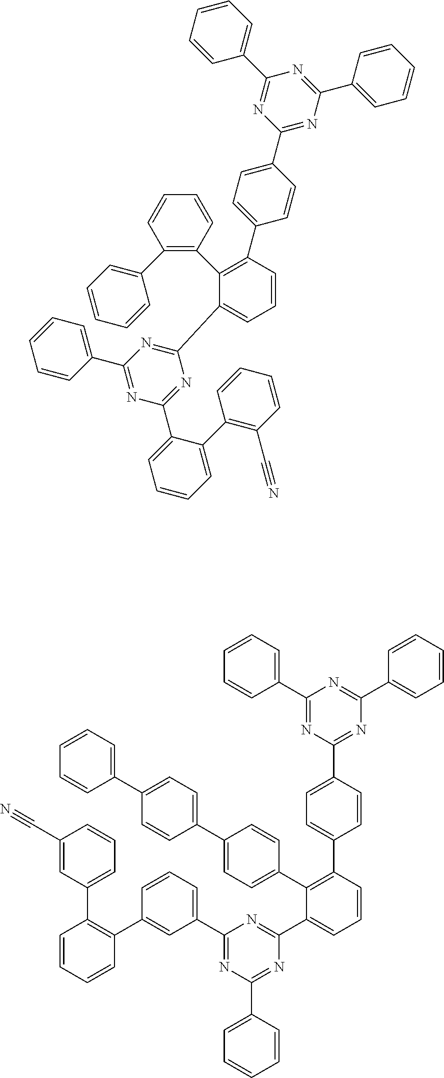 [SMILES: N#Cc1ccccc1-c1ccccc1-c1nc(-c2ccccc2)nc(-c2cccc(-c3ccc(-c4nc(-c5ccccc5)nc(-c5ccccc5)n4)cc3)c2-c2ccccc2-c2ccccc2)n1 ; N#Cc1cccc(-c2ccccc2-c2cccc(-c3nc(-c4ccccc4)nc(-c4cccc(-c5ccc(-c6nc(-c7ccccc7)nc(-c7ccccc7)n6)cc5)c4-c4ccc(-c5ccc(-c6ccccc6)cc5)cc4)n3)c2)c1]

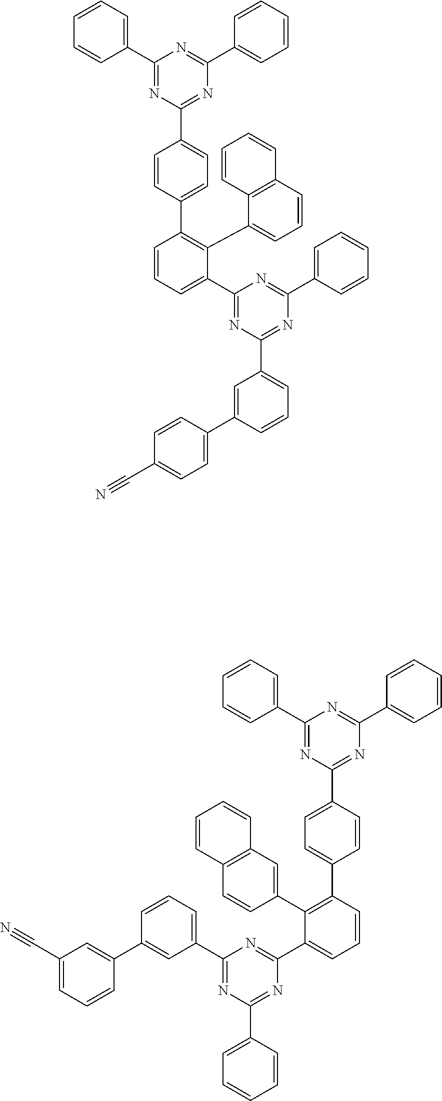 [SMILES: N#Cc1ccc(-c2cccc(-c3nc(-c4ccccc4)nc(-c4cccc(-c5ccc(-c6nc(-c7ccccc7)nc(-c7ccccc7)n6)cc5)c4-c4cccc5ccccc45)n3)c2)cc1 ; N#Cc1cccc(-c2cccc(-c3nc(-c4ccccc4)nc(-c4cccc(-c5ccc(-c6nc(-c7ccccc7)nc(-c7ccccc7)n6)cc5)c4-c4ccc5ccccc5c4)n3)c2)c1]

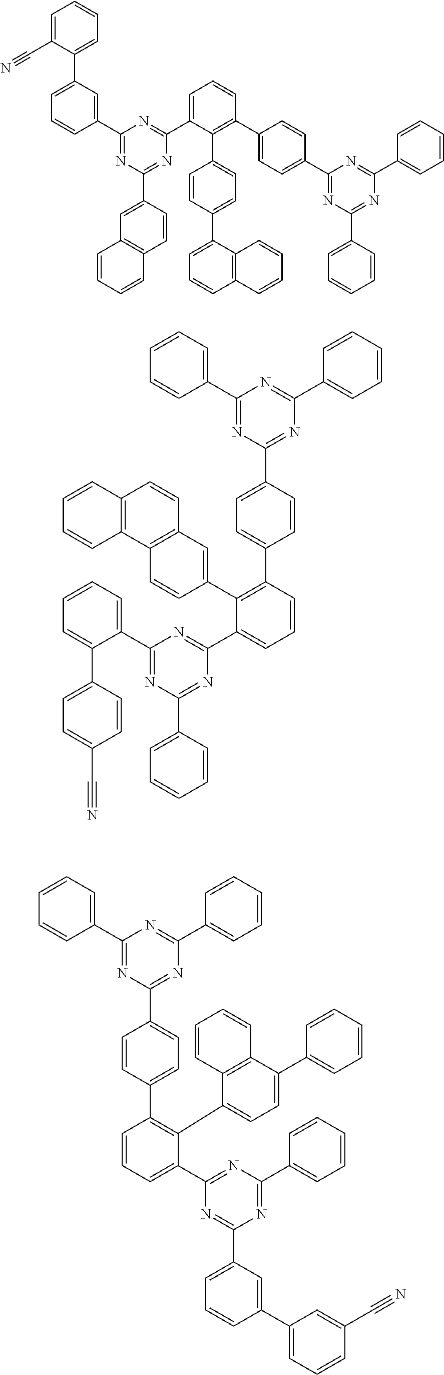

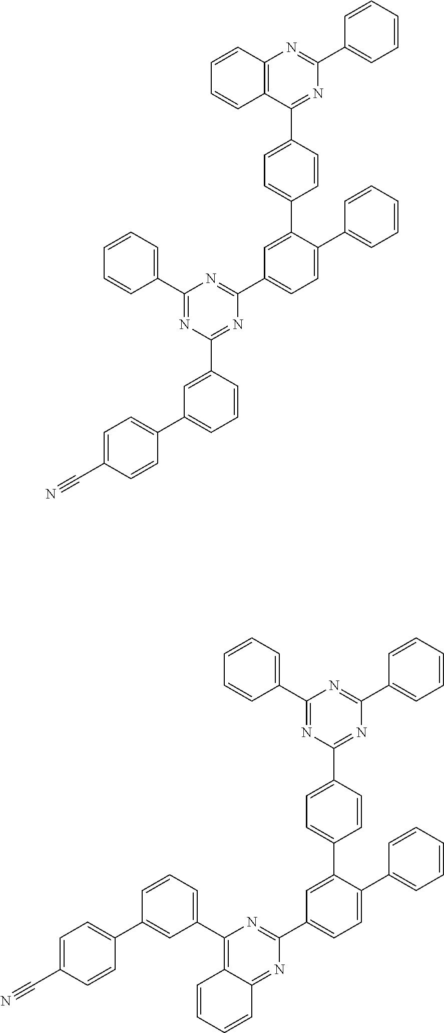 [SMILES: N#Cc1ccc(-c2cccc(-c3nc(-c4ccccc4)nc(-c4ccc(-c5ccccc5)c(-c5ccc(-c6nc(-c7ccccc7)nc7ccccc67)cc5)c4)n3)c2)cc1 ; N#Cc1ccc(-c2cccc(-c3nc(-c4ccc(-c5ccccc5)c(-c5ccc(-c6nc(-c7ccccc7)nc(-c7ccccc7)n6)cc5)c4)nc4ccccc34)c2)cc1]

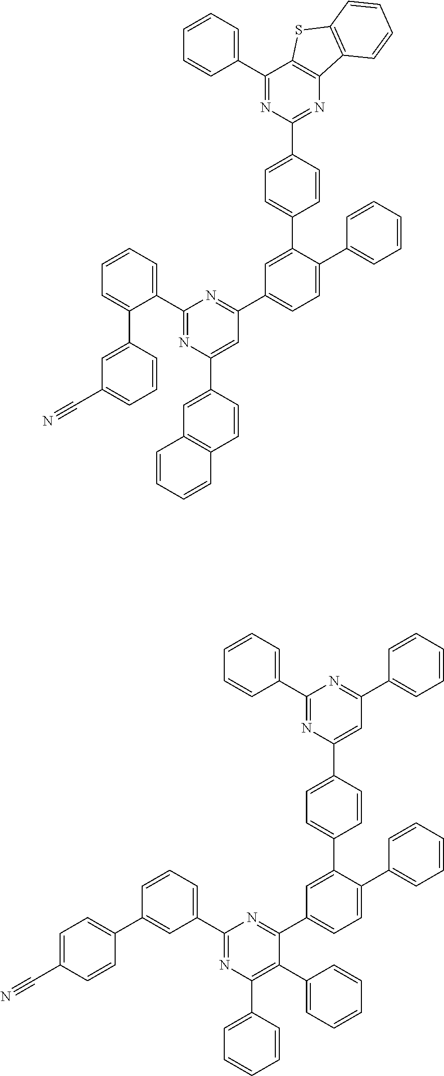 [SMILES: N#Cc1cccc(-c2ccccc2-c2nc(-c3ccc(-c4ccccc4)c(-c4ccc(-c5nc(-c6ccccc6)c6sc7ccccc7c6n5)cc4)c3)cc(-c3ccc4ccccc4c3)n2)c1 ; N#Cc1ccc(-c2cccc(-c3nc(-c4ccccc4)c(-c4ccccc4)c(-c4ccc(-c5ccccc5)c(-c5ccc(-c6cc(-c7ccccc7)nc(-c7ccccc7)n6)cc5)c4)n3)c2)cc1]

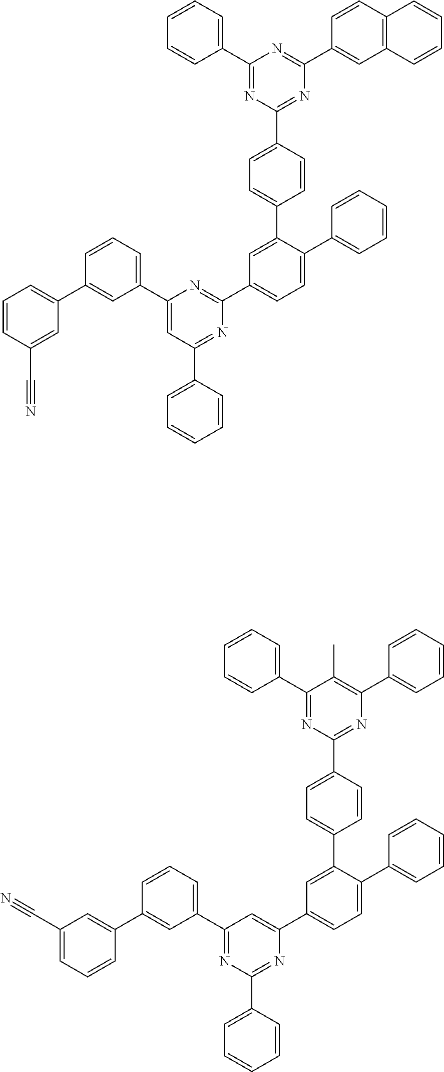 [SMILES: N#Cc1cccc(-c2cccc(-c3cc(-c4ccccc4)nc(-c4ccc(-c5ccccc5)c(-c5ccc(-c6nc(-c7ccccc7)nc(-c7ccc8ccccc8c7)n6)cc5)c4)n3)c2)c1 ; Cc1c(-c2ccccc2)nc(-c2ccc(-c3cc(-c4cc(-c5cccc(-c6cccc(C#N)c6)c5)nc(-c5ccccc5)n4)ccc3-c3ccccc3)cc2)nc1-c1ccccc1]

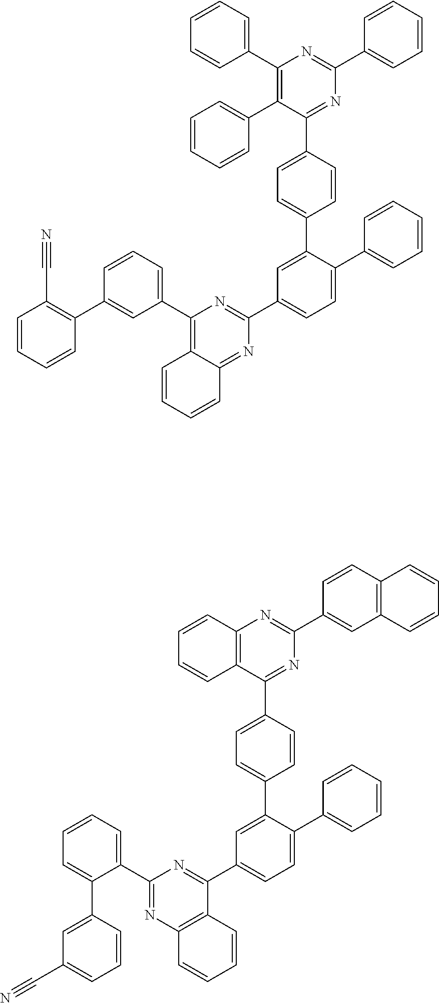 [SMILES: N#Cc1ccccc1-c1cccc(-c2nc(-c3ccc(-c4ccccc4)c(-c4ccc(-c5nc(-c6ccccc6)nc(-c6ccccc6)c5-c5ccccc5)cc4)c3)nc3ccccc23)c1 ; N#Cc1cccc(-c2ccccc2-c2nc(-c3ccc(-c4ccccc4)c(-c4ccc(-c5nc(-c6ccc7ccccc7c6)nc6ccccc56)cc4)c3)c3ccccc3n2)c1]

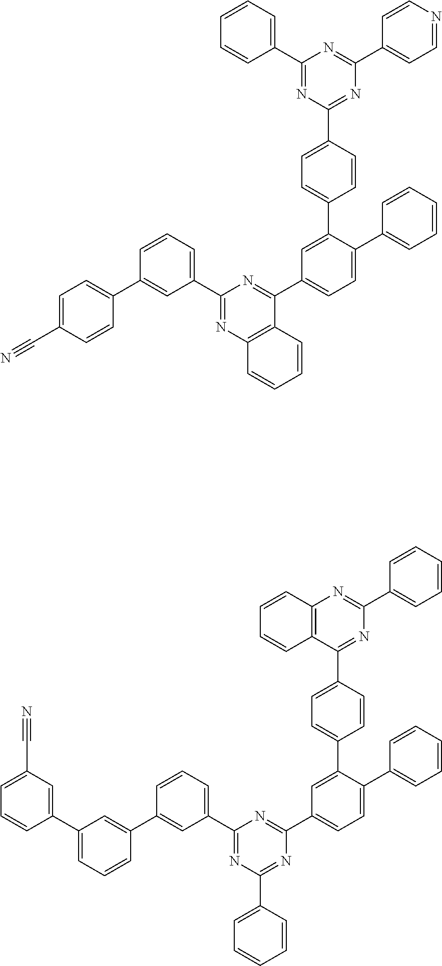 [SMILES: N#Cc1ccc(-c2cccc(-c3nc(-c4ccc(-c5ccccc5)c(-c5ccc(-c6nc(-c7ccccc7)nc(-c7ccncc7)n6)cc5)c4)c4ccccc4n3)c2)cc1 ; N#Cc1cccc(-c2cccc(-c3cccc(-c4nc(-c5ccccc5)nc(-c5ccc(-c6ccccc6)c(-c6ccc(-c7nc(-c8ccccc8)nc8ccccc78)cc6)c5)n4)c3)c2)c1]

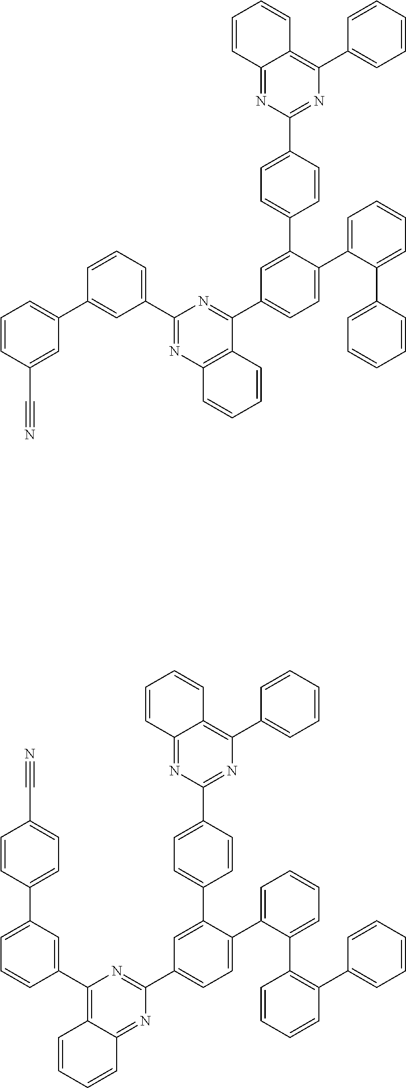 [SMILES: N#Cc1cccc(-c2cccc(-c3nc(-c4ccc(-c5ccccc5-c5ccccc5)c(-c5ccc(-c6nc(-c7ccccc7)c7ccccc7n6)cc5)c4)c4ccccc4n3)c2)c1 ; N#Cc1ccc(-c2cccc(-c3nc(-c4ccc(-c5ccccc5-c5ccccc5-c5ccccc5)c(-c5ccc(-c6nc(-c7ccccc7)c7ccccc7n6)cc5)c4)nc4ccccc34)c2)cc1]

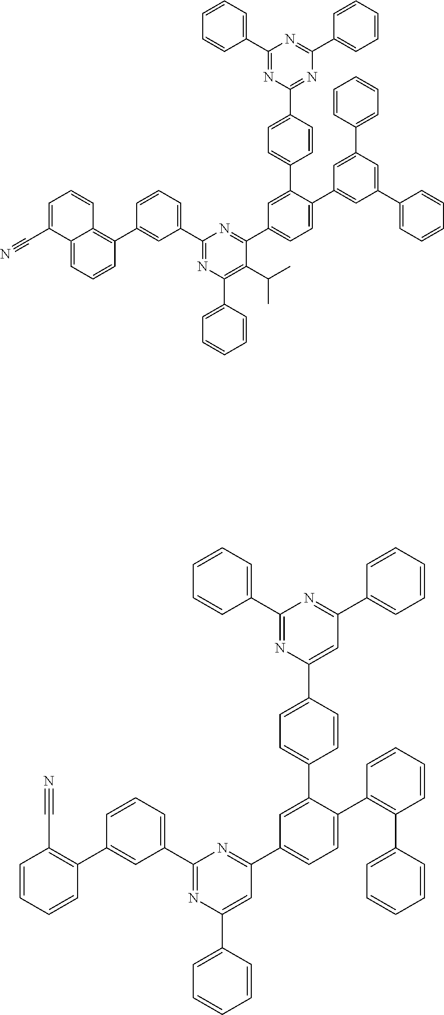 [SMILES: CC(C)c1c(-c2ccccc2)nc(-c2cccc(-c3cccc4c(C#N)cccc34)c2)nc1-c1ccc(-c2cc(-c3ccccc3)cc(-c3ccccc3)c2)c(-c2ccc(-c3nc(-c4ccccc4)nc(-c4ccccc4)n3)cc2)c1 ; N#Cc1ccccc1-c1cccc(-c2nc(-c3ccccc3)cc(-c3ccc(-c4ccccc4-c4ccccc4)c(-c4ccc(-c5cc(-c6ccccc6)nc(-c6ccccc6)n5)cc4)c3)n2)c1]

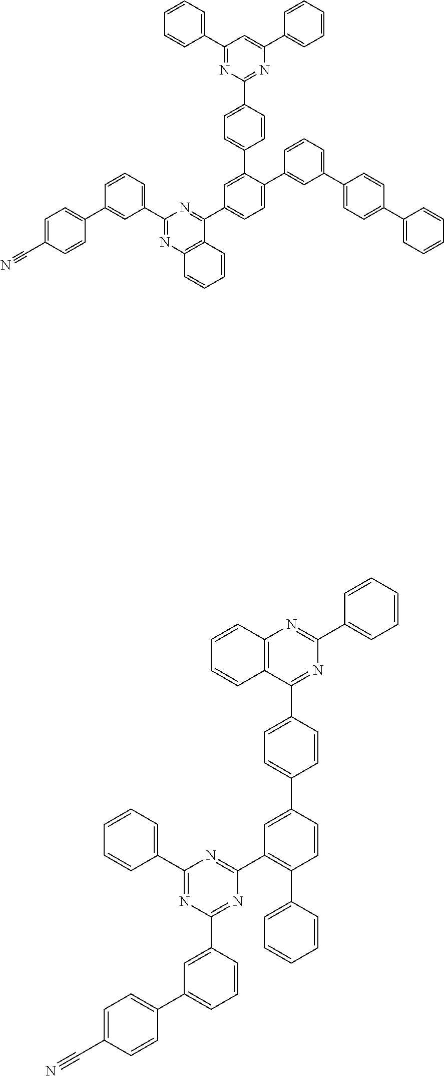 [SMILES: N#Cc1ccc(-c2cccc(-c3nc(-c4ccc(-c5cccc(-c6ccc(-c7ccccc7)cc6)c5)c(-c5ccc(-c6nc(-c7ccccc7)cc(-c7ccccc7)n6)cc5)c4)c4ccccc4n3)c2)cc1 ; N#Cc1ccc(-c2cccc(-c3nc(-c4ccccc4)nc(-c4cc(-c5ccc(-c6nc(-c7ccccc7)nc7ccccc67)cc5)ccc4-c4ccccc4)n3)c2)cc1]

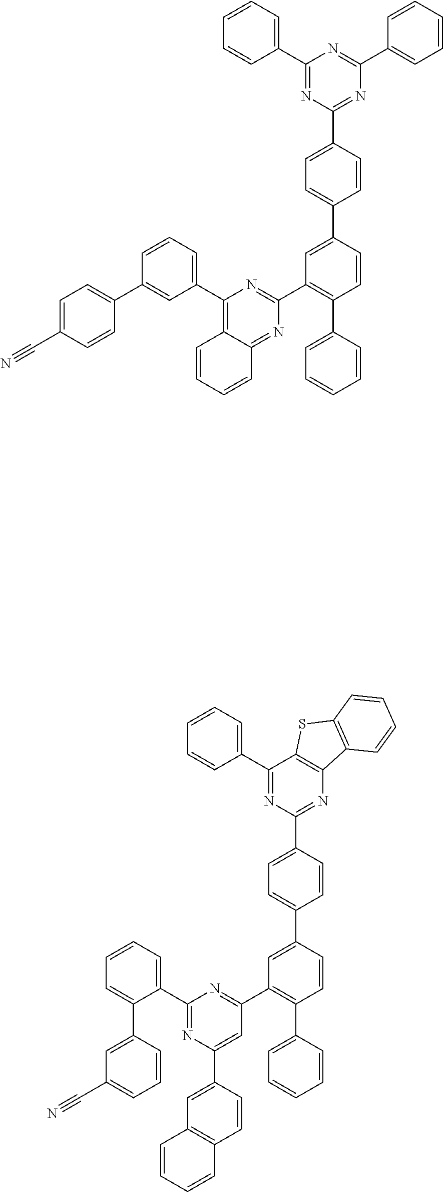 [SMILES: N#Cc1ccc(-c2cccc(-c3nc(-c4cc(-c5ccc(-c6nc(-c7ccccc7)nc(-c7ccccc7)n6)cc5)ccc4-c4ccccc4)nc4ccccc34)c2)cc1 ; N#Cc1cccc(-c2ccccc2-c2nc(-c3ccc4ccccc4c3)cc(-c3cc(-c4ccc(-c5nc(-c6ccccc6)c6sc7ccccc7c6n5)cc4)ccc3-c3ccccc3)n2)c1]

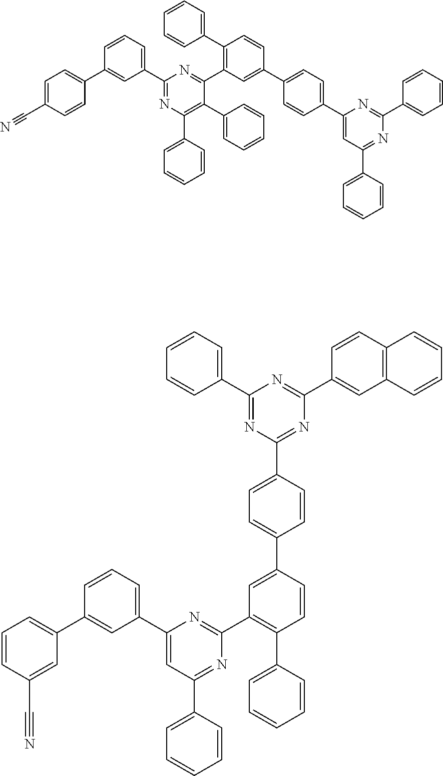 [SMILES: N#Cc1ccc(-c2cccc(-c3nc(-c4ccccc4)c(-c4ccccc4)c(-c4cc(-c5ccc(-c6cc(-c7ccccc7)nc(-c7ccccc7)n6)cc5)ccc4-c4ccccc4)n3)c2)cc1 ; N#Cc1cccc(-c2cccc(-c3cc(-c4ccccc4)nc(-c4cc(-c5ccc(-c6nc(-c7ccccc7)nc(-c7ccc8ccccc8c7)n6)cc5)ccc4-c4ccccc4)n3)c2)c1]

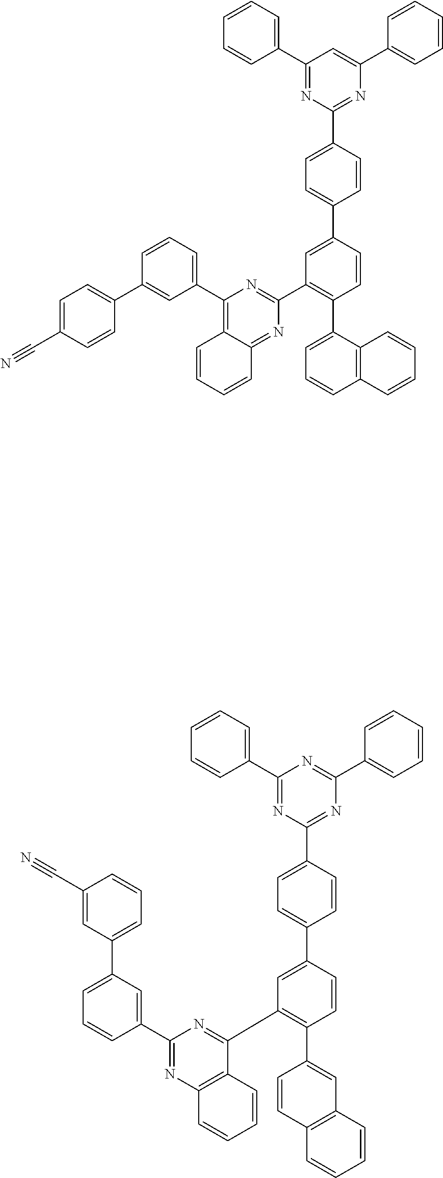 [SMILES: N#Cc1ccc(-c2cccc(-c3nc(-c4cc(-c5ccc(-c6nc(-c7ccccc7)cc(-c7ccccc7)n6)cc5)ccc4-c4cccc5ccccc45)nc4ccccc34)c2)cc1 ; N#Cc1cccc(-c2cccc(-c3nc(-c4cc(-c5ccc(-c6nc(-c7ccccc7)nc(-c7ccccc7)n6)cc5)ccc4-c4ccc5ccccc5c4)c4ccccc4n3)c2)c1]

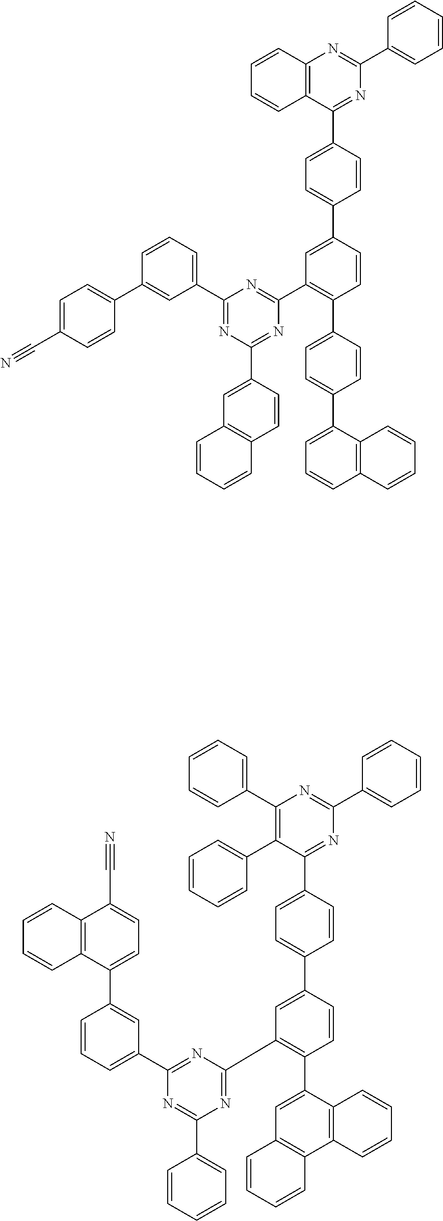 [SMILES: N#Cc1ccc(-c2cccc(-c3nc(-c4ccc5ccccc5c4)nc(-c4cc(-c5ccc(-c6nc(-c7ccccc7)nc7ccccc67)cc5)ccc4-c4ccc(-c5cccc6ccccc56)cc4)n3)c2)cc1 ; N#Cc1ccc(-c2cccc(-c3nc(-c4ccccc4)nc(-c4cc(-c5ccc(-c6nc(-c7ccccc7)nc(-c7ccccc7)c6-c6ccccc6)cc5)ccc4-c4cc5ccccc5c5ccccc45)n3)c2)c2ccccc12]

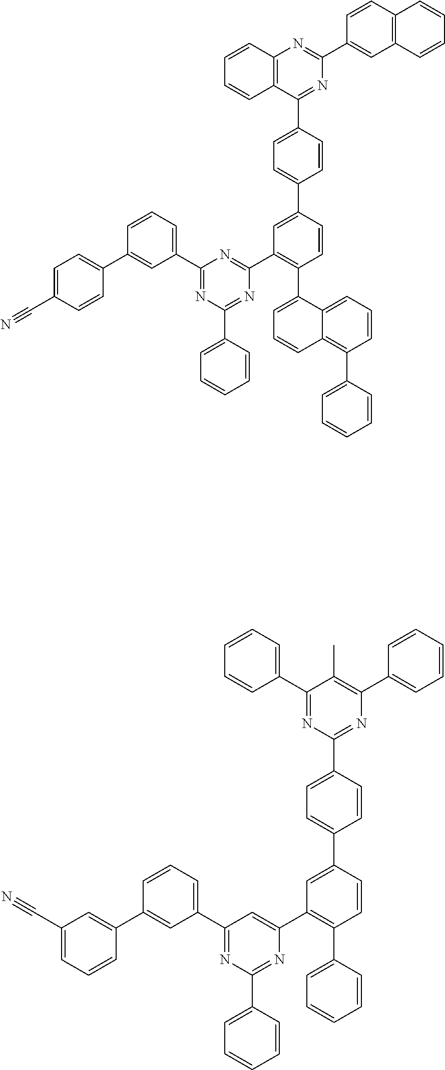 [SMILES: N#Cc1ccc(-c2cccc(-c3nc(-c4ccccc4)nc(-c4cc(-c5ccc(-c6nc(-c7ccc8ccccc8c7)nc7ccccc67)cc5)ccc4-c4cccc5c(-c6ccccc6)cccc45)n3)c2)cc1 ; Cc1c(-c2ccccc2)nc(-c2ccc(-c3ccc(-c4ccccc4)c(-c4cc(-c5cccc(-c6cccc(C#N)c6)c5)nc(-c5ccccc5)n4)c3)cc2)nc1-c1ccccc1]

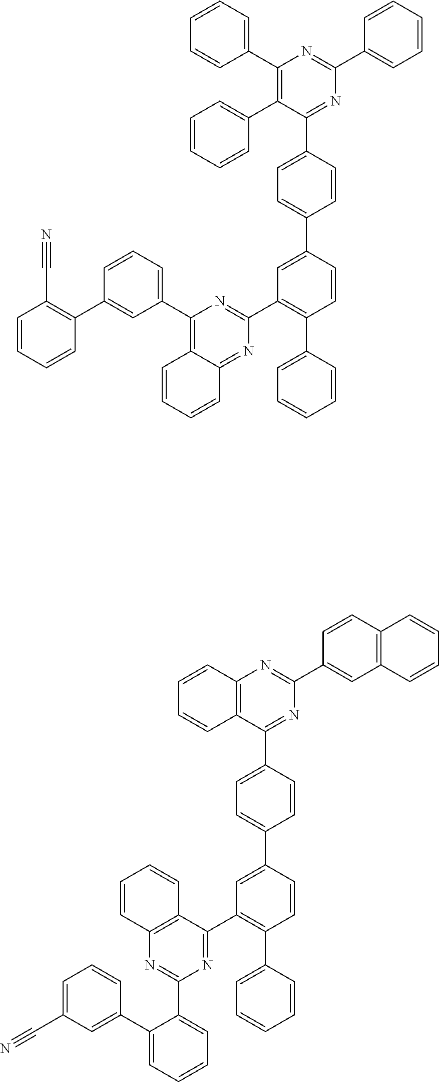 [SMILES: N#Cc1ccccc1-c1cccc(-c2nc(-c3cc(-c4ccc(-c5nc(-c6ccccc6)nc(-c6ccccc6)c5-c5ccccc5)cc4)ccc3-c3ccccc3)nc3ccccc23)c1 ; N#Cc1cccc(-c2ccccc2-c2nc(-c3cc(-c4ccc(-c5nc(-c6ccc7ccccc7c6)nc6ccccc56)cc4)ccc3-c3ccccc3)c3ccccc3n2)c1]

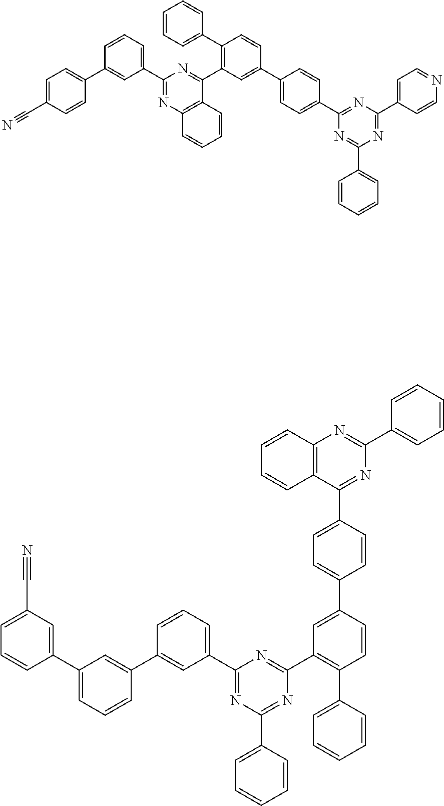 [SMILES: N#Cc1ccc(-c2cccc(-c3nc(-c4cc(-c5ccc(-c6nc(-c7ccccc7)nc(-c7ccncc7)n6)cc5)ccc4-c4ccccc4)c4ccccc4n3)c2)cc1 ; N#Cc1cccc(-c2cccc(-c3cccc(-c4nc(-c5ccccc5)nc(-c5cc(-c6ccc(-c7nc(-c8ccccc8)nc8ccccc78)cc6)ccc5-c5ccccc5)n4)c3)c2)c1]

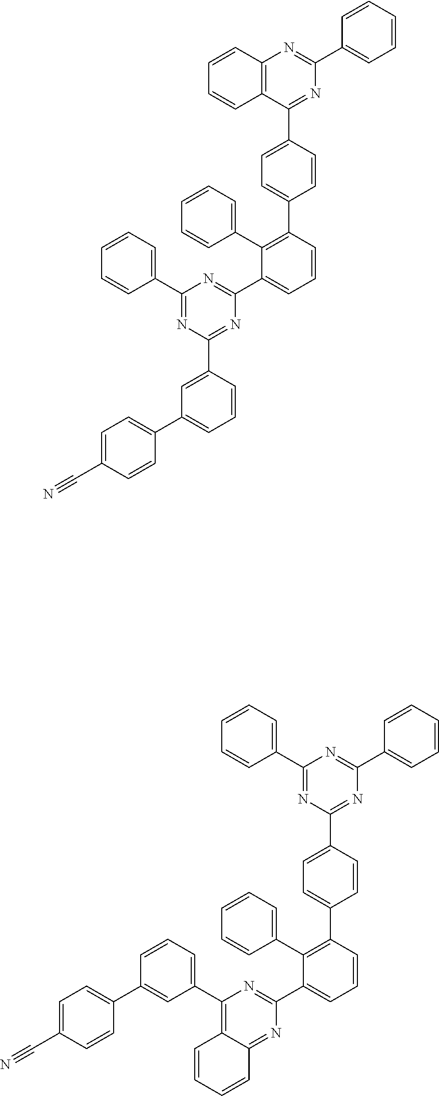 [SMILES: N#Cc1ccc(-c2cccc(-c3nc(-c4ccccc4)nc(-c4cccc(-c5ccc(-c6nc(-c7ccccc7)nc7ccccc67)cc5)c4-c4ccccc4)n3)c2)cc1 ; N#Cc1ccc(-c2cccc(-c3nc(-c4cccc(-c5ccc(-c6nc(-c7ccccc7)nc(-c7ccccc7)n6)cc5)c4-c4ccccc4)nc4ccccc34)c2)cc1]

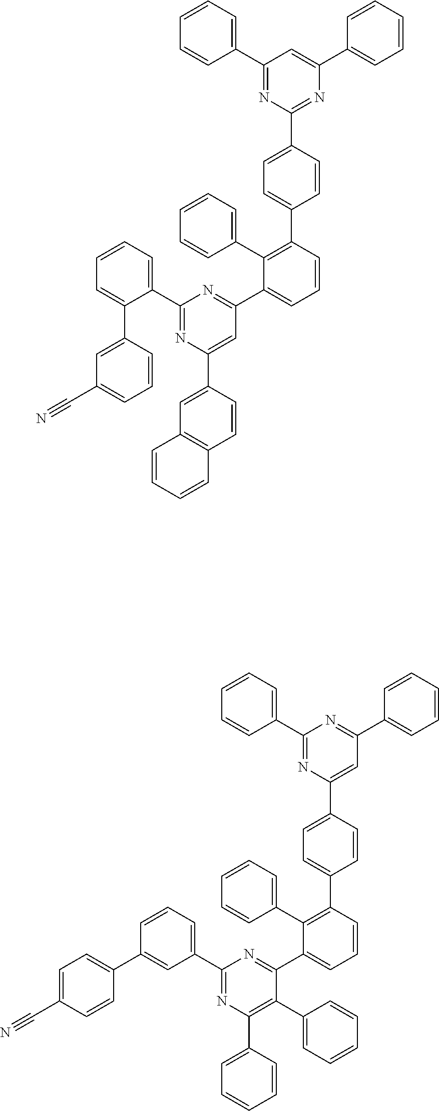 [SMILES: N#Cc1cccc(-c2ccccc2-c2nc(-c3ccc4ccccc4c3)cc(-c3cccc(-c4ccc(-c5nc(-c6ccccc6)cc(-c6ccccc6)n5)cc4)c3-c3ccccc3)n2)c1 ; N#Cc1ccc(-c2cccc(-c3nc(-c4ccccc4)c(-c4ccccc4)c(-c4cccc(-c5ccc(-c6cc(-c7ccccc7)nc(-c7ccccc7)n6)cc5)c4-c4ccccc4)n3)c2)cc1]

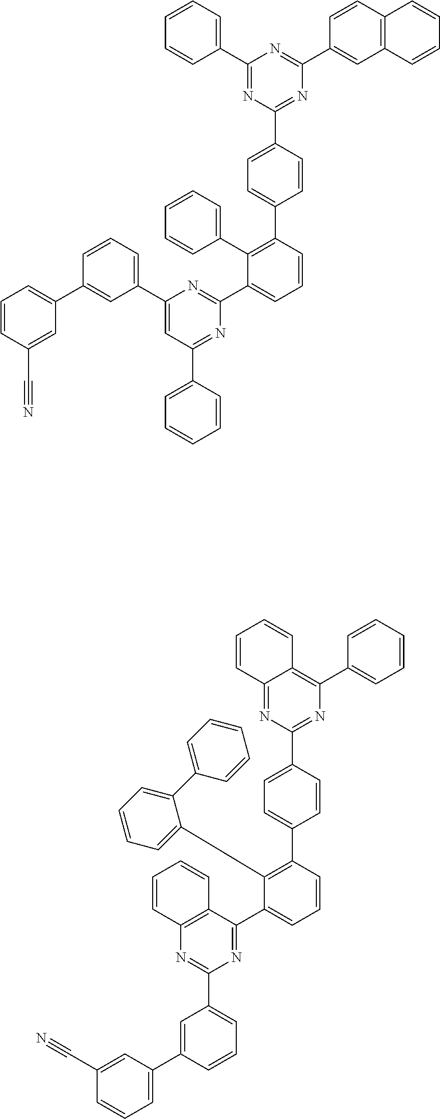 [SMILES: N#Cc1cccc(-c2cccc(-c3cc(-c4ccccc4)nc(-c4cccc(-c5ccc(-c6nc(-c7ccccc7)nc(-c7ccc8ccccc8c7)n6)cc5)c4-c4ccccc4)n3)c2)c1 ; N#Cc1cccc(-c2cccc(-c3nc(-c4cccc(-c5ccc(-c6nc(-c7ccccc7)c7ccccc7n6)cc5)c4-c4ccccc4-c4ccccc4)c4ccccc4n3)c2)c1]

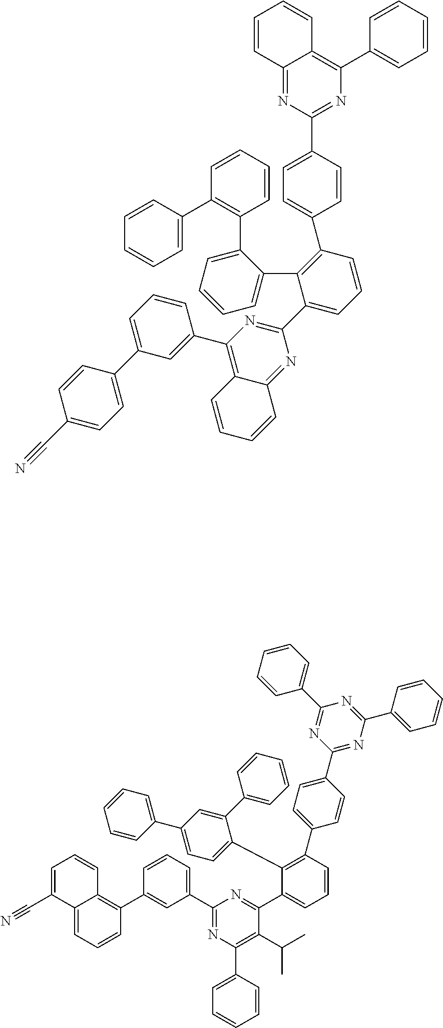 [SMILES: N#Cc1ccc(-c2cccc(-c3nc(-c4cccc(-c5ccc(-c6nc(-c7ccccc7)c7ccccc7n6)cc5)c4-c4ccccc4-c4ccccc4-c4ccccc4)nc4ccccc34)c2)cc1 ; CC(C)c1c(-c2ccccc2)nc(-c2cccc(-c3cccc4c(C#N)cccc34)c2)nc1-c1cccc(-c2ccc(-c3nc(-c4ccccc4)nc(-c4ccccc4)n3)cc2)c1-c1ccc(-c2ccccc2)cc1-c1ccccc1]

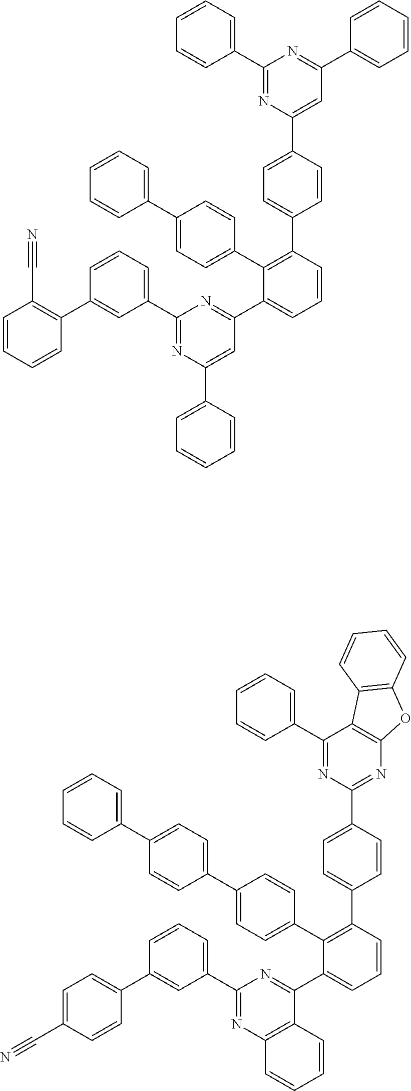 [SMILES: N#Cc1ccccc1-c1cccc(-c2nc(-c3ccccc3)cc(-c3cccc(-c4ccc(-c5cc(-c6ccccc6)nc(-c6ccccc6)n5)cc4)c3-c3ccc(-c4ccccc4)cc3)n2)c1 ; N#Cc1ccc(-c2cccc(-c3nc(-c4cccc(-c5ccc(-c6nc(-c7ccccc7)c7c(n6)oc6ccccc67)cc5)c4-c4ccc(-c5ccc(-c6ccccc6)cc5)cc4)c4ccccc4n3)c2)cc1]

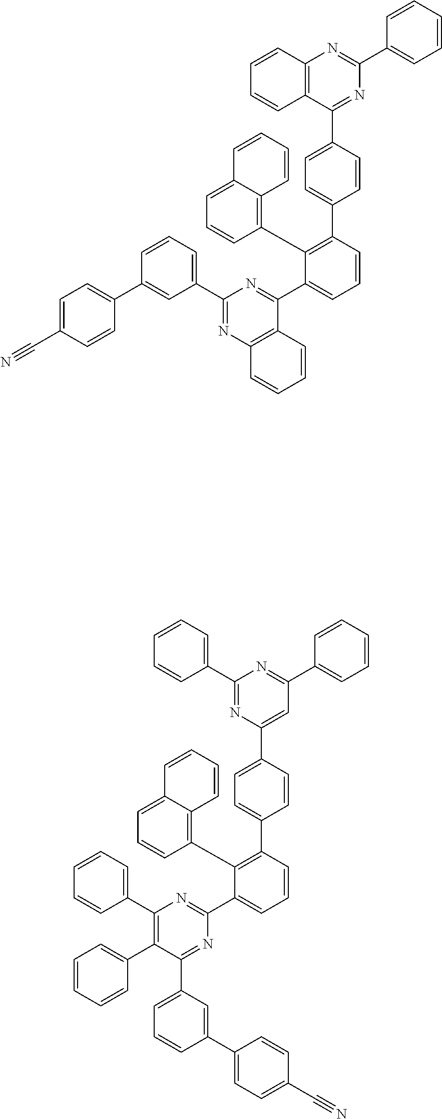 [SMILES: N#Cc1ccc(-c2cccc(-c3nc(-c4cccc(-c5ccc(-c6nc(-c7ccccc7)nc7ccccc67)cc5)c4-c4cccc5ccccc45)c4ccccc4n3)c2)cc1 ; N#Cc1ccc(-c2cccc(-c3nc(-c4cccc(-c5ccc(-c6cc(-c7ccccc7)nc(-c7ccccc7)n6)cc5)c4-c4cccc5ccccc45)nc(-c4ccccc4)c3-c3ccccc3)c2)cc1]

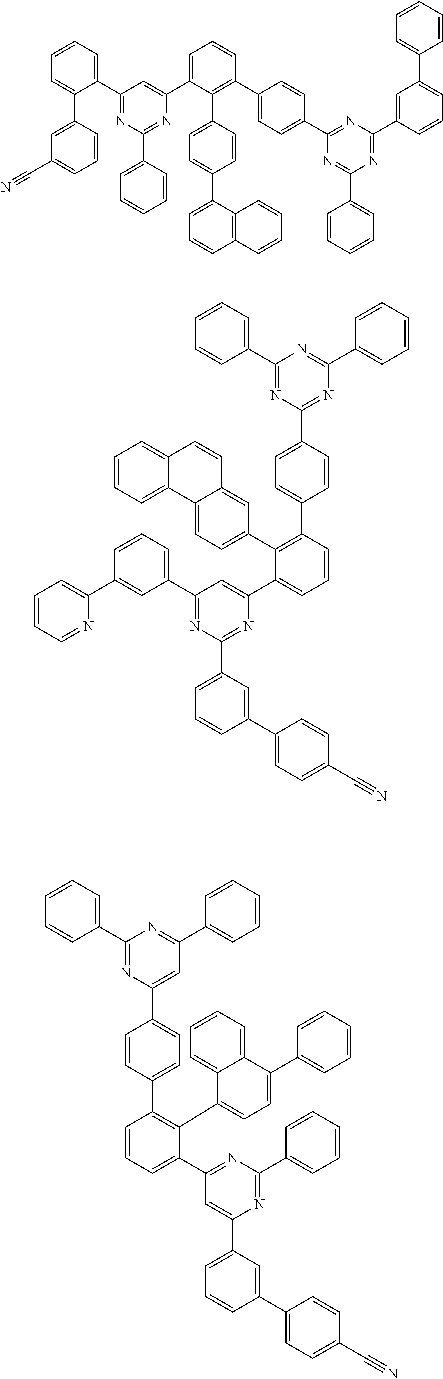 [SMILES: N#Cc1cccc(-c2ccccc2-c2cc(-c3cccc(-c4ccc(-c5nc(-c6ccccc6)nc(-c6cccc(-c7ccccc7)c6)n5)cc4)c3-c3ccc(-c4cccc5ccccc45)cc3)nc(-c3ccccc3)n2)c1 ; N#Cc1ccc(-c2cccc(-c3nc(-c4cccc(-c5ccccn5)c4)cc(-c4cccc(-c5ccc(-c6nc(-c7ccccc7)nc(-c7ccccc7)n6)cc5)c4-c4ccc5c(ccc6ccccc65)c4)n3)c2)cc1 ; N#Cc1ccc(-c2cccc(-c3cc(-c4cccc(-c5ccc(-c6cc(-c7ccccc7)nc(-c7ccccc7)n6)cc5)c4-c4ccc(-c5ccccc5)c5ccccc45)nc(-c4ccccc4)n3)c2)cc1]

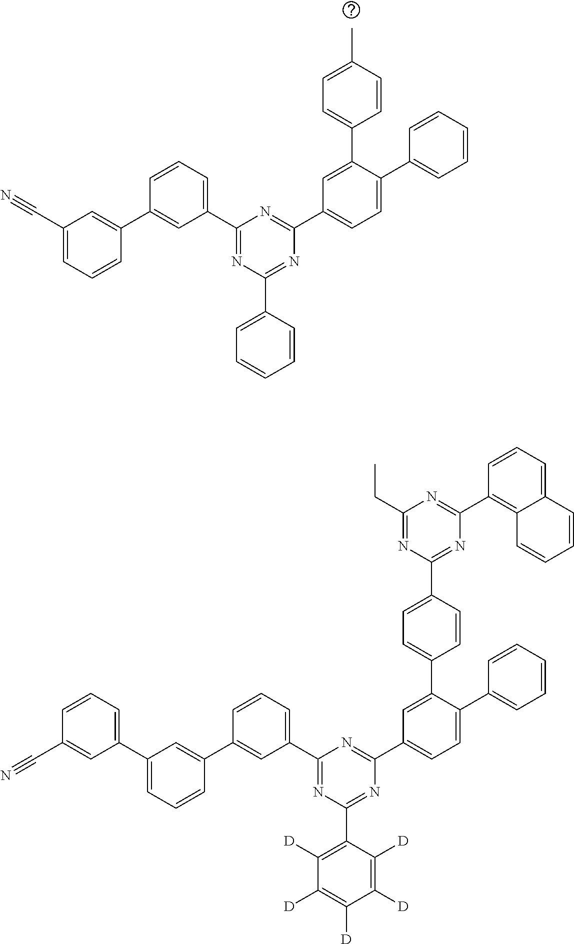 [SMILES: Cc1ccc(-c2cc(-c3nc(-c4ccccc4)nc(-c4cccc(-c5cccc(C#N)c5)c4)n3)ccc2-c2ccccc2)cc1 ; [2H]c1c([2H])c([2H])c(-c2nc(-c3cccc(-c4cccc(-c5cccc(C#N)c5)c4)c3)nc(-c3ccc(-c4ccccc4)c(-c4ccc(-c5nc(CC)nc(-c6cccc7ccccc67)n5)cc4)c3)n2)c([2H])c1[2H]]

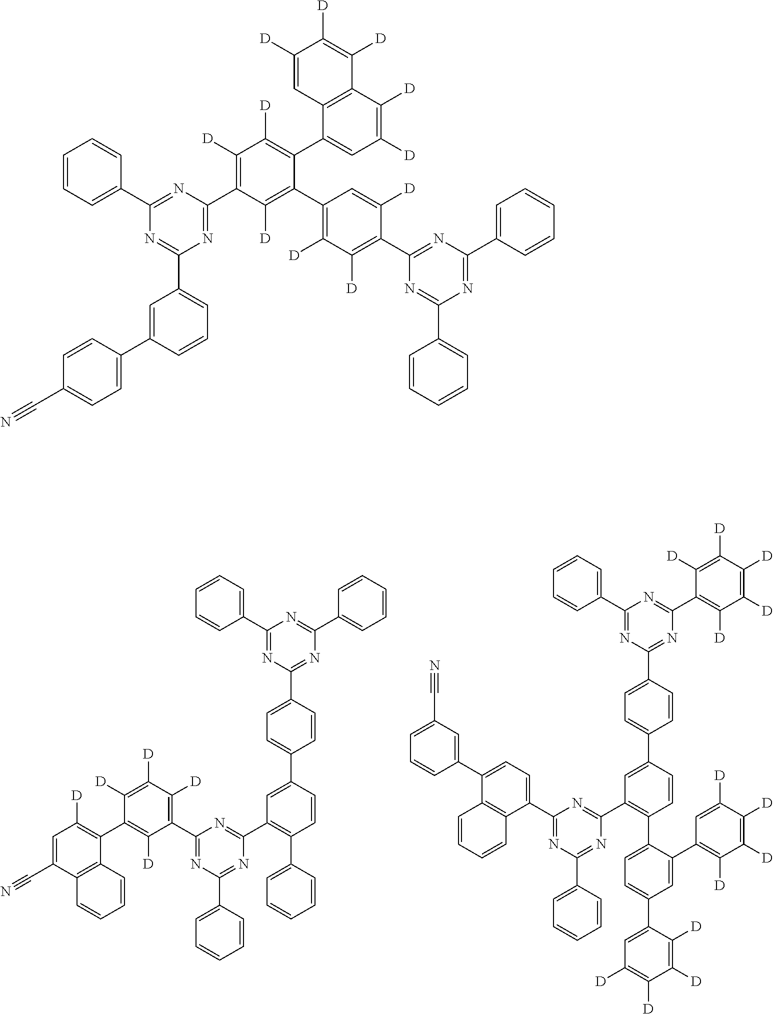

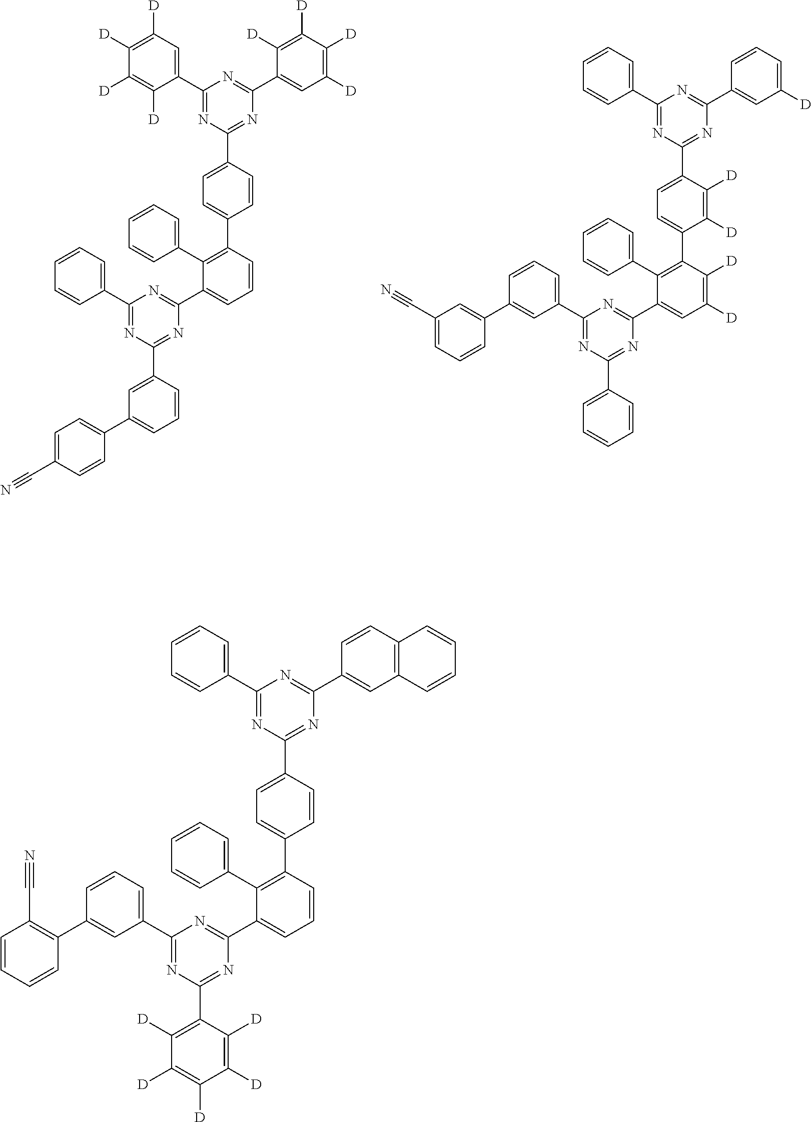 [SMILES: [2H]c1cc(-c2nc(-c3ccc(-c4cccc(-c5nc(-c6ccccc6)nc(-c6cccc(-c7ccc(C#N)cc7)c6)n5)c4-c4ccccc4)cc3)nc(-c3cc([2H])c([2H])c([2H])c3[2H])n2)c([2H])c([2H])c1[2H] ; [2H]c1cccc(-c2nc(-c3ccccc3)nc(-c3ccc(-c4c([2H])c([2H])cc(-c5nc(-c6ccccc6)nc(-c6cccc(-c7cccc(C#N)c7)c6)n5)c4-c4ccccc4)c([2H])c3[2H])n2)c1 ; [2H]c1c([2H])c([2H])c(-c2nc(-c3cccc(-c4ccccc4C#N)c3)nc(-c3cccc(-c4ccc(-c5nc(-c6ccccc6)nc(-c6ccc7ccccc7c6)n5)cc4)c3-c3ccccc3)n2)c([2H])c1[2H]]

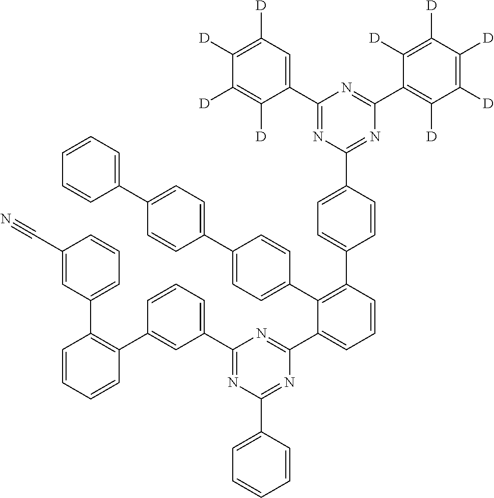 [SMILES: [2H]c1cc(-c2nc(-c3ccc(-c4cccc(-c5nc(-c6ccccc6)nc(-c6cccc(-c7ccccc7-c7cccc(C#N)c7)c6)n5)c4-c4ccc(-c5ccc(-c6ccccc6)cc5)cc4)cc3)nc(-c3c([2H])c([2H])c([2H])c([2H])c3[2H])n2)c([2H])c([2H])c1[2H]]

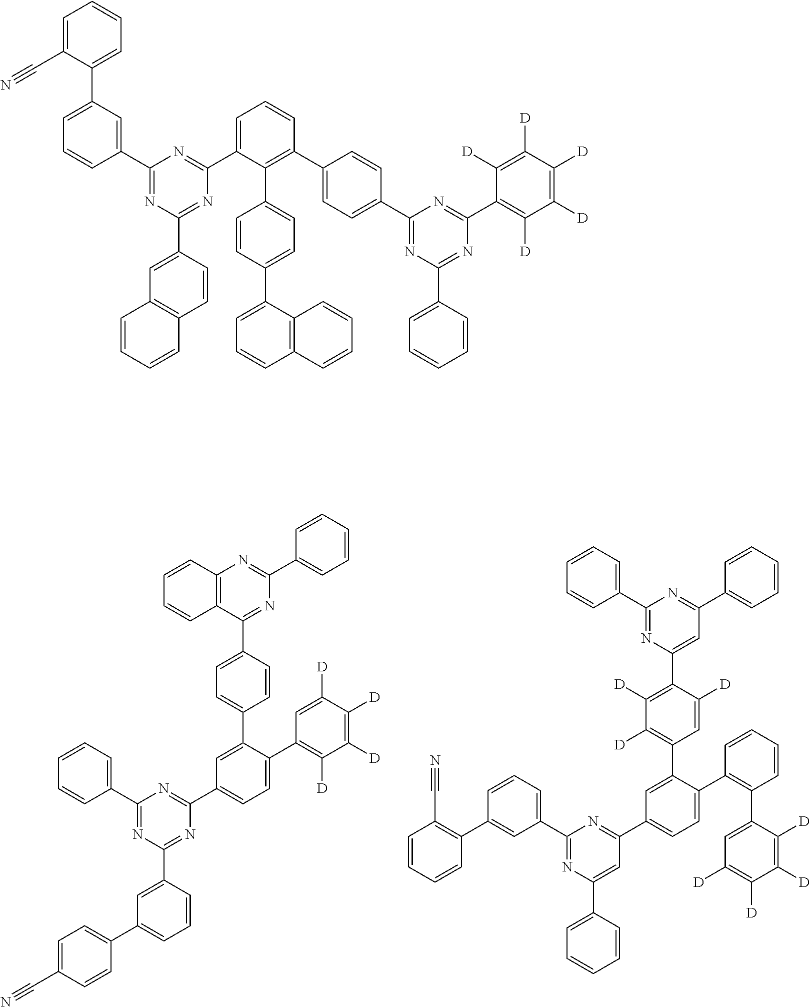 [SMILES: [2H]c1c([2H])c([2H])c(-c2nc(-c3ccccc3)nc(-c3ccc(-c4cccc(-c5nc(-c6cccc(-c7ccccc7C#N)c6)nc(-c6ccc7ccccc7c6)n5)c4-c4ccc(-c5cccc6ccccc56)cc4)cc3)n2)c([2H])c1[2H] ; [2H]c1cc(-c2ccc(-c3nc(-c4ccccc4)nc(-c4cccc(-c5ccc(C#N)cc5)c4)n3)cc2-c2ccc(-c3nc(-c4ccccc4)nc4ccccc34)cc2)c([2H])c([2H])c1[2H] ; [2H]c1cc(-c2ccccc2-c2ccc(-c3cc(-c4ccccc4)nc(-c4cccc(-c5ccccc5C#N)c4)n3)cc2-c2cc([2H])c(-c3cc(-c4ccccc4)nc(-c4ccccc4)n3)c([2H])c2[2H])c([2H])c([2H])c1[2H]]

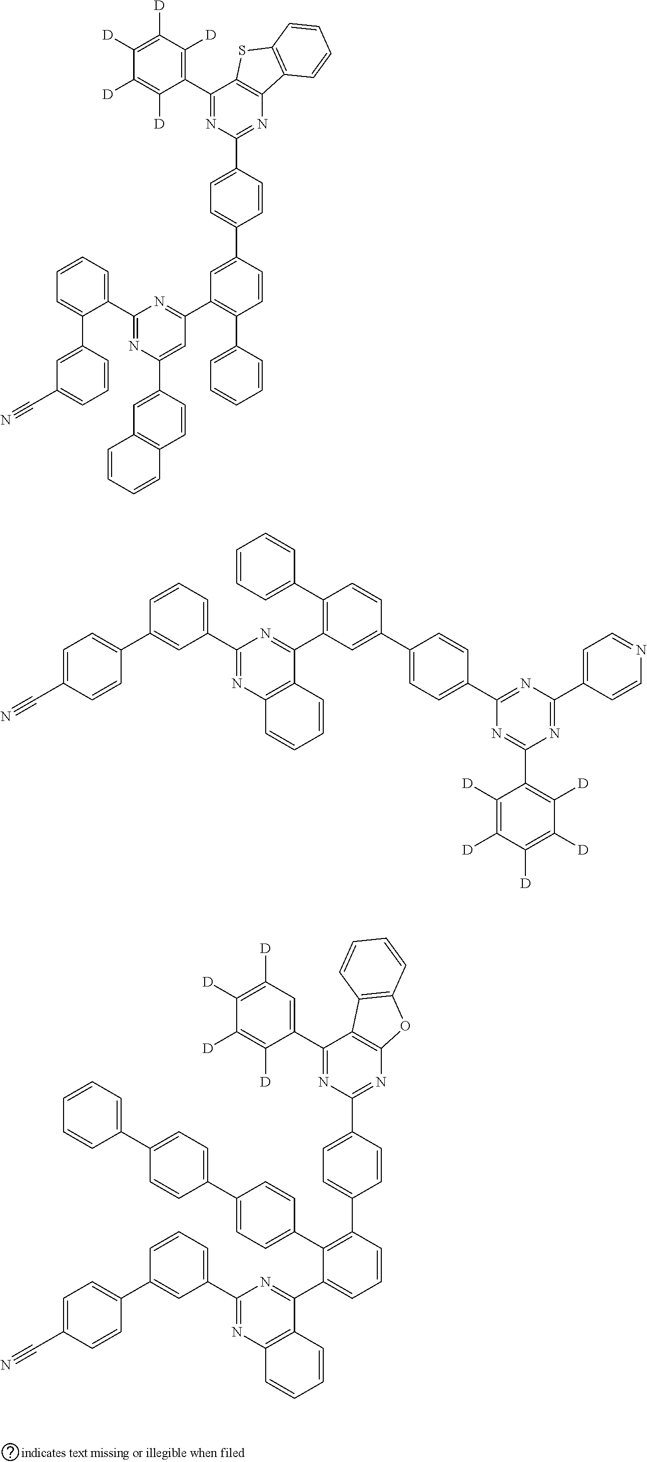 [SMILES: [2H]c1c([2H])c([2H])c(-c2nc(-c3ccc(-c4ccc(-c5ccccc5)c(-c5cc(-c6ccc7ccccc7c6)nc(-c6ccccc6-c6cccc(C#N)c6)n5)c4)cc3)nc3c2sc2ccccc23)c([2H])c1[2H] ; [2H]c1c([2H])c([2H])c(-c2nc(-c3ccncc3)nc(-c3ccc(-c4ccc(-c5ccccc5)c(-c5nc(-c6cccc(-c7ccc(C#N)cc7)c6)nc6ccccc56)c4)cc3)n2)c([2H])c1[2H] ; [2H]c1cc(-c2nc(-c3ccc(-c4cccc(-c5nc(-c6cccc(-c7ccc(C#N)cc7)c6)nc6ccccc56)c4-c4ccc(-c5ccc(-c6ccccc6)cc5)cc4)cc3)nc3oc4ccccc4c23)c([2H])c([2H])c1[2H]]

According to an exemplary embodiment of the present specification, the deuterium substitution rate of Chemical Formula 1 is 300 or more.

According to an exemplary embodiment of the present specification, the deuterium substitution rate of Chemical Formula 1 is 40% or more.

According to an exemplary embodiment of the present specification, the deuterium substitution rate of Chemical Formula 1 is 50% or more.

According to an exemplary embodiment of the present specification, the deuterium substitution rate of Chemical Formula 1 is 60% or more.

According to an exemplary embodiment of the present specification, the deuterium substitution rate of Chemical Formula 1 is 70% or more.

According to an exemplary embodiment of the present specification, the deuterium substitution rate of Chemical Formula 1 is 80% or more.

According to an exemplary embodiment of the present specification, the deuterium substitution rate of Chemical Formula 1 is 90% or more.

According to an exemplary embodiment of the present specification, the deuterium substitution rate of Chemical Formula 1 is 100%.

The substituent of the compound of Chemical Formula 1 may be bonded by a method known in the art, and the type and position of the substituent or the number of substituents may be changed according to the technology known in the art.

In addition, various substituents may be introduced into the core structure having the structure described above to synthesize compounds having inherent characteristics of the introduced substituents. For example, a substituent usually used for a hole injection layer material, a material for transporting holes, a light emitting layer material, and an electron transport layer material, which are used for manufacturing an organic light emitting device, may be introduced into the core structure to synthesize a material which satisfies conditions required for each organic material layer.

Furthermore, the organic light emitting device according to the present invention is an organic light emitting device including: a first electrode; a second electrode provided to face the first electrode; and an organic material layer having one or more layers provided between the first electrode and the second electrode, in which one or more layers of the organic material layer include the above-described compound.

The organic light emitting device of the present invention may be manufactured using typical manufacturing methods and materials of an organic light emitting device, except that the above-described compound is used to form an organic material layer having one or more layers.

The compound may be formed as an organic material layer by not only a vacuum deposition method, but also a solution application method when an organic light emitting device is manufactured. Herein, the solution coating method means spin coating, dip coating, inkjet printing, screen printing, a spray method, roll coating, and the like, but is not limited thereto.

The organic material layer of the organic light emitting device of the present invention may be composed of a single-layered structure, but may be composed of a multi-layered structure in which two or more organic material layers are stacked. For example, the organic light emitting device of the present invention may have a structure including a hole injection layer, a hole transport layer, a layer which injects and transports holes simultaneously, a light emitting layer, an electron transport layer, an electron injection layer, and the like as organic material layers. However, the structure of the organic light emitting device is not limited thereto, and may include a fewer or greater number of organic material layers.

In the organic light emitting device of the present invention, the organic material layer may include one or more layers of an electron transport layer, an electron injection layer, and an electron injection and transport layer, and one or more layers of the layers may include the compound represented by Chemical Formula 1.

In another organic light emitting device, the organic material layer may include an electron transport layer or an electron injection layer, and the electron transport layer or the electron injection layer may include the compound represented by Chemical Formula 1.

In the organic light emitting device of the present invention, the electron injection and transport layer includes the compound of Chemical Formula 1 and a metal complex.

In the organic light emitting device of the present invention, the organic material layer may include one or more layers of a hole injection layer, a hole transport layer, and a layer which injects and transports holes simultaneously, and one or more layers of the layers may include the compound represented by Chemical Formula 1.

In still another organic light emitting device, the organic material layer may include a hole injection layer or a hole transport layer, and the hole transport layer or the hole injection layer may include the compound represented by Chemical Formula 1.

In an exemplary embodiment of the present specification, the first electrode is an anode, and the second electrode is a cathode.

According to another exemplary embodiment, the first electrode is a cathode, and the second electrode is an anode.
(1) Anode/Hole transport layer/Light emitting layer/Cathode
(2) Anode/Hole injection layer/Hole transport layer/Light emitting layer/Cathode
(3) Anode/Hole injection layer/Hole buffer layer/Hole transport layer/Light emitting layer/Cathode
(4) Anode/Hole transport layer/Light emitting layer/Electron transport layer/Cathode
(5) Anode/Hole transport layer/Light emitting layer/Electron transport layer/Electron injection layer/Cathode
(6) Anode/Hole injection layer/Hole transport layer/Light emitting layer/Electron transport layer/Cathode
(7) Anode/Hole injection layer/Hole transport layer/Light emitting layer/Electron transport layer/Electron injection layer/Cathode
(8) Anode/Hole injection layer/Hole buffer layer/Hole transport layer/Light emitting layer/Electron transport layer/Cathode
(9) Anode/Hole injection layer/Hole buffer layer/Hole transport layer/Light emitting layer/Electron transport layer/Electron injection layer/Cathode
(10) Anode/Hole transport layer/Electron blocking layer/Light emitting layer/Electron transport layer/Cathode
(11) Anode/Hole transport layer/Electron blocking layer/Light emitting layer/Electron transport layer/Electron injection layer/Cathode
(12) Anode/Hole injection layer/Hole transport layer/Electron blocking layer/Light emitting layer/Electron transport layer/Cathode
(13) Anode/Hole injection layer/Hole transport layer/Electron blocking layer/Light emitting layer/Electron transport layer/Electron injection layer/Cathode
(14) Anode/Hole transport layer/Light emitting layer/Hole blocking layer/Electron transport layer/Cathode
(15) Anode/Hole transport layer/Light emitting layer/Hole blocking layer/Electron transport layer/Electron injection layer/Cathode
(16) Anode/Hole injection layer/Hole transport layer/Light emitting layer/Hole blocking layer/Electron transport layer/Cathode
(17) Anode/Hole injection layer/Hole transport layer/Light emitting layer/Hole blocking layer/Electron transport layer/Electron injection layer/Cathode
(18) Anode/Hole injection layer/Hole transport layer/Electron blocking layer/Light emitting layer/Hole blocking layer/Electron injection and transport layer/Cathode
(19) Anode/Hole injection layer/Hole transport layer/Light emitting layer/Electron injection and transport layer/Cathode
(20) Anode/Hole injection layer/First hole transport layer/Second hole transport layer/Light emitting layer/Electron injection and transport layer/Cathode

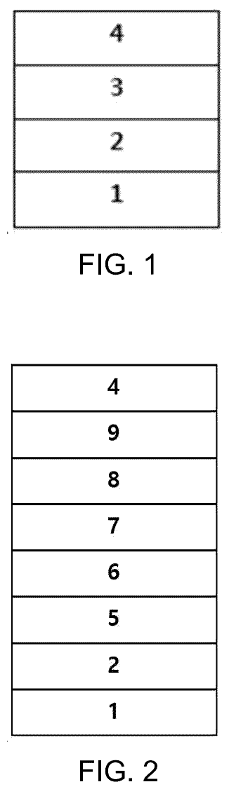

The structure of the organic light emitting device of the present invention may have a structure as illustrated in FIGS. 1 to 3, but is not limited thereto.

FIG. 1 exemplifies the structure of an organic light emitting device in which an anode 2, a light emitting layer 3, and a cathode 4 are sequentially stacked on a substrate 1. In the structure described above, the compound represented by Chemical Formula 1 may be included in the organic material layer 3.

FIG. 2 exemplifies the structure of an organic light emitting device in which an anode 2, a hole injection layer 5, a hole transport layer 6, an electron blocking layer 7, a light emitting layer 8, an electron injection and transport layer 9, and a cathode 4 are sequentially stacked on a substrate 1. The compound represented by Chemical Formula 1 may be included in the electron injection and transport layer 9.

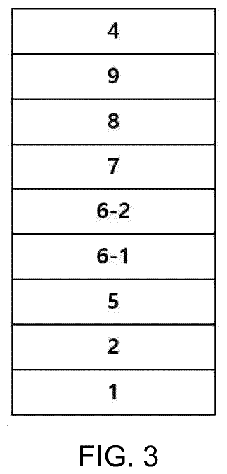

FIG. 3 exemplifies the structure of an organic light emitting device in which an anode 2, a hole injection layer 5, a first hole transport layer 6-1, a second hole transport layer 6-2, an electron blocking layer 7, a light emitting layer 8, an electron injection and transport layer 9, and a cathode 4 are sequentially stacked on a substrate 1. The compound represented by Chemical Formula 1 may be included in the electron injection and transport layer 9.

For example, the organic light emitting device according to the present invention may be manufactured by depositing a metal or a metal oxide having conductivity, or an alloy thereof on a substrate to form an anode, forming an organic material layer having one or more layers selected from the group consisting of a hole injection layer, a hole transport layer, a layer which transports and injects holes simultaneously, a light emitting layer, an electron transport layer, an electron injection layer, and a layer which transports and injects electrons simultaneously, thereon, and then depositing a material, which may be used as a cathode, thereon, by using a physical vapor deposition (PVD) method such as sputtering or e-beam evaporation. In addition to the method described above, an organic light emitting device may be made by sequentially depositing a cathode material, an organic material layer, and an anode material on a substrate.

The organic material layer may have a multi-layered structure including a hole injection layer, a hole transport layer, a light emitting layer, and an electron transport layer, and the like, but is not limited thereto and may have a single-layered structure. Further, the organic material layer may be manufactured to include a fewer number of layers by a method such as a solvent process, for example, spin coating, dip coating, doctor blading, screen printing, inkjet printing, or a thermal transfer method, using various polymer materials, instead of a deposition method.

The anode is an electrode which injects holes, and as an anode material, materials having a high work function are usually preferred so as to facilitate the injection of holes into an organic material layer. Specific examples of the anode material which may be used in the present invention include: a metal, such as vanadium, chromium, copper, zinc, and gold, or an alloy thereof; a metal oxide, such as zinc oxide, indium oxide, indium tin oxide (ITO), and indium zinc oxide (IZO); a combination of a metal and an oxide, such as ZnO:Al or SnO₂:Sb; a conductive polymer, such as poly(3-methylthiophene), poly[3,4-(ethylene-1,2-dioxy)thiophene](PEDOT), polypyrrole, and polyaniline; and the like, but are not limited thereto.

The cathode is an electrode which injects electrons, and as a cathode material, materials having a low work function are usually preferred so as to facilitate the injection of electrons into an organic material layer. Specific examples of the cathode material include: a metal such as magnesium, calcium, sodium, potassium, titanium, indium, yttrium, lithium, gadolinium, aluminum, silver, tin, and lead, or an alloy thereof; a multi-layer structured material, such as LiF/Al or LiO₂/Al; and the like, but are not limited thereto.

The hole injection layer is a layer which serves to facilitate the injection of holes from an anode to a light emitting layer, and a hole injection material is preferably a material which may proficiently accept holes from an anode at a low voltage, and the highest occupied molecular orbital (HOMO) of the hole injection material is preferably a value between the work function of the anode material and the HOMO of the neighboring organic material layer. Specific examples of the hole injection material include metal porphyrin, oligothiophene, arylamine-based organic materials, hexanitrile hexaazatriphenylene-based organic materials, quinacridone-based organic materials, perylene-based organic materials, anthraquinone, polyaniline-based and polythiophene-based conductive polymers, and the like, but are not limited thereto. The hole injection layer may have a thickness of 1 to 150 nm. When the hole injection layer has a thickness of 1 nm or more, there is an advantage in that it is possible to prevent hole injection characteristics from deteriorating, and when the hole injection layer has a thickness of 150 nm or less, there is an advantage in that it is possible to prevent the driving voltage from being increased in order to improve the movement of holes due to the too thick hole injection layer.

According to an exemplary embodiment of the present specification, the hole injection layer may be a compound of the following Chemical Formula HI-1.

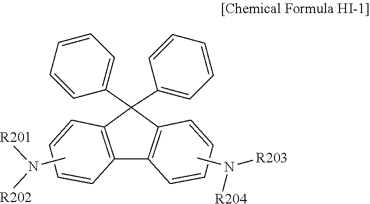

In Chemical Formula HI-1,
R201 to R204 are the same as or different from each other, and are each independently hydrogen; deuterium; a substituted or unsubstituted alkyl group; a substituted or unsubstituted aryl group; or a substituted or unsubstituted heteroaryl group, or are bonded to an adjacent group to form a substituted or unsubstituted ring, and
in an exemplary embodiment of the present specification, R201 to R204 are the same as or different from each other, and are a substituted or unsubstituted alkyl group; a substituted or unsubstituted aryl group; or a substituted or unsubstituted heteroaryl group, or are bonded to an adjacent group to form a substituted or unsubstituted ring.

In an exemplary embodiment of the present specification, R201 to R204 are the same as or different from each other, and are each independently hydrogen; deuterium; a substituted or unsubstituted alkyl group; a substituted or unsubstituted aryl group; or a substituted or unsubstituted heteroaryl group.

In an exemplary embodiment of the present specification, R201 to R204 are the same as or different from each other, and are each independently a substituted or unsubstituted alkyl group; a substituted or unsubstituted aryl group; or a substituted or unsubstituted heteroaryl group.

In an exemplary embodiment of the present specification, R201 to R204 are the same as or different from each other, and are each independently a substituted or unsubstituted aryl group; or a substituted or unsubstituted heteroaryl group.

In an exemplary embodiment of the present specification, R202 and R204 are the same as or different from each other, and are each independently a substituted or unsubstituted aryl group.

In an exemplary embodiment of the present specification, R201 and R203 are the same as or different from each other, and are each independently a substituted or unsubstituted aryl group having 6 to 30 carbon atoms.

In an exemplary embodiment of the present specification, R201 and R203 are the same as or different from each other, and are each independently a substituted or unsubstituted aryl group having 6 to 20 carbon atoms.

In an exemplary embodiment of the present specification, R201 and R203 are the same as or different from each other, and are each independently a substituted or unsubstituted phenyl group.

In an exemplary embodiment of the present specification, R202 and R204 are the same as or different from each other, and are each independently a substituted or unsubstituted heteroaryl group.

In an exemplary embodiment of the present specification, R202 and R204 are the same as or different from each other, and are each independently a substituted or unsubstituted heteroaryl group having 2 to 20 carbon atoms.

In an exemplary embodiment of the present specification, R202 and R204 are the same as or different from each other, and are each independently a substituted or unsubstituted heteroaryl group having 2 to 10 carbon atoms.

In an exemplary embodiment of the present specification, R202 and R204 are the same as or different from each other, and are each independently a substituted or unsubstituted dibenzofuranyl group; a substituted or unsubstituted dibenzothiophenyl group; or a substituted or unsubstituted carbazolyl group.

In an exemplary embodiment of the present specification, R202 and R204 are the same as or different from each other, and are each independently a carbazolyl group that is unsubstituted or substituted with an aryl group.

In an exemplary embodiment of the present specification, R202 and R204 are the same as or different from each other, and are each independently a carbazolyl group that is unsubstituted or substituted with a phenyl group.

In an exemplary embodiment of the present specification, Chemical Formula HI-1 is represented by the following compound.

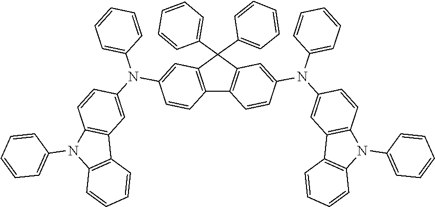 [SMILES: c1ccc(N(c2ccc3c(c2)C(c2ccccc2)(c2ccccc2)c2cc(N(c4ccccc4)c4ccc5c(c4)c4ccccc4n5-c4ccccc4)ccc2-3)c2ccc3c(c2)c2ccccc2n3-c2ccccc2)cc1]

The hole transport layer may perform a role of smoothly transporting holes. A hole transport material is suitably a material having high hole mobility which may accept holes from an anode or a hole injection layer and transfer the holes to a light emitting layer. Specific examples thereof include arylamine-based organic materials, conductive polymers, block copolymers having both conjugated portions and non-conjugated portions, and the like, but are not limited thereto.

According to an exemplary embodiment of the present specification, the hole transport layer includes a compound of the following Chemical Formula HT-1, but is not limited thereto.

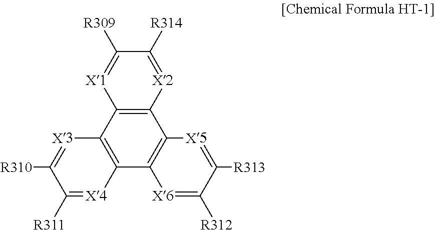

In Chemical Formula HT-1,
at least one of X′1 to X′6 is N, and the others are CH, and
R309 to R314 are the same as or different from each other, and are each independently hydrogen; deuterium; a cyano group; a substituted or unsubstituted alkyl group; a substituted or unsubstituted amine group; a substituted or unsubstituted aryl group; or a substituted or unsubstituted heteroaryl group, or are bonded to an adjacent group to form a substituted or unsubstituted ring.

According to an exemplary embodiment of the present specification, X′1 to X′6 are N.

According to an exemplary embodiment of the present specification, R309 to R314 are a cyano group.

According to an exemplary embodiment of the present specification, Chemical Formula HT-1 may include the following compound.

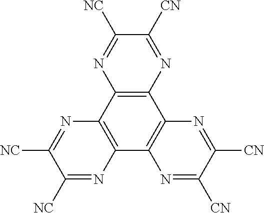 [SMILES: N#Cc1nc2c3nc(C#N)c(C#N)nc3c3nc(C#N)c(C#N)nc3c2nc1C#N]

According to an exemplary embodiment of the present specification, the hole transport layer includes a compound represented by the following Chemical Formula HT-2, but is not limited thereto.

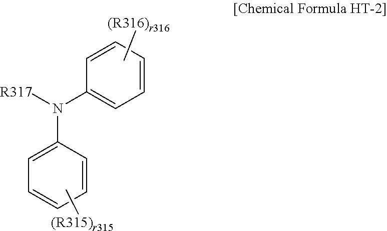

In Chemical Formula HT-2,
R315 to R317 are the same as or different from each other, and are each independently any one selected from the group consisting of hydrogen; deuterium; a substituted or unsubstituted alkyl group; a substituted or unsubstituted aryl group; a substituted or unsubstituted heteroaryl group; and a combination thereof, or are bonded to an adjacent group to form a substituted or unsubstituted ring,
r315 is an integer from 1 to 5, and when r315 is 2 or higher, two or more R315's are the same as or different from each other, and
r316 is an integer from 1 to 5, and when r316 is 2 or higher, two or more R316's are the same as or different from each other.

According to an exemplary embodiment of the present specification, R317 is any one selected from the group consisting of a substituted or unsubstituted aryl group; a substituted or unsubstituted heteroaryl group; and a combination thereof.

According to an exemplary embodiment of the present specification, R317 is a substituted or unsubstituted phenyl group; or a substituted or unsubstituted fluorene group, or adjacent groups are bonded to each other to form a substituted or unsubstituted ring.

According to an exemplary embodiment of the present specification, R317 is a fluorene group that is unsubstituted or substituted with an alkyl group or an aryl group.

According to an exemplary embodiment of the present specification, R315 and R316 are the same as or different from each other, and are each independently hydrogen; deuterium; or a substituted or unsubstituted carbazole group.

According to an exemplary embodiment of the present specification, R315 and R316 are the same as or different from each other, and are each independently hydrogen; deuterium; a phenyl group; or a carbazole group that is unsubstituted or substituted with a biphenyl group.

According to an exemplary embodiment of the present specification, Chemical Formula HT-2 is represented by the following compound.

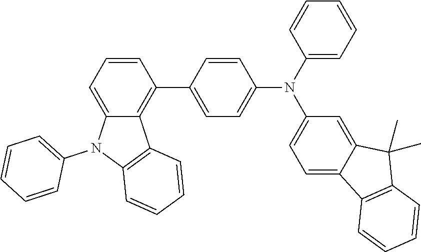 [SMILES: CC1(C)c2ccccc2-c2ccc(N(c3ccccc3)c3ccc(-c4cccc5c4c4ccccc4n5-c4ccccc4)cc3)cc21]

According to an exemplary embodiment of the present specification, the hole transport layer may include the compound of Chemical Formula HT-1 and/or the compound of Chemical Formula HT-2.

According to an exemplary embodiment of the present specification, the hole transport layer may consist of a first hole transport layer and a second hole transport layer.

According to an exemplary embodiment of the present specification, the first hole transport layer may include the compound of Chemical Formula HT-1.

According to an exemplary embodiment of the present specification, the second hole transport layer may include the compound of Chemical Formula HT-2.

An electron blocking layer may be provided between a hole transport layer and a light emitting layer. As the electron blocking layer, the above-described spiro compound or a material known in the art may be used.

The light emitting layer may emit red, green, or blue light, and may be composed of a phosphorescent material or a fluorescent material. The light emitting material is a material which may receive holes and electrons from a hole transport layer and an electron transport layer, respectively, and combine the holes and the electrons to emit light in a visible ray region, and is preferably a material having high quantum efficiency for fluorescence or phosphorescence. Specific examples thereof include: 8-hydroxy-quinoline aluminum complexes (Alq₃); carbazole-based compounds; dimerized styryl compounds; BAlq; 10-hydroxybenzoquinoline-metal compounds; benzoxazole-based, benzothiazole-based and benzimidazole-based compounds; poly(p-phenylenevinylene) (PPV)-based polymers; spiro compounds; polyfluorene, rubrene, and the like, but are not limited thereto.

Examples of the host material for the light emitting layer include condensed aromatic ring derivatives, or hetero ring-containing compounds, and the like. Specifically, examples of the condensed aromatic ring derivative include anthracene derivatives, pyrene derivatives, naphthalene derivatives, pentacene derivatives, phenanthrene compounds, fluoranthene compounds, and the like, and examples of the hetero ring-containing compound include carbazole derivatives, dibenzofuran derivatives, ladder-type furan compounds, pyrimidine derivatives, and the like, but the examples thereof are not limited thereto.

According to an exemplary embodiment of the present specification, the host includes a compound of the following Chemical Formula H-1, but is not limited thereto.

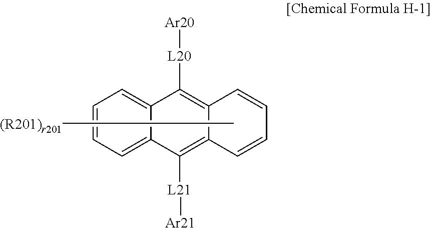

In Chemical Formula H-1,
L20 and L21 are the same as or different from each other, and are each independently a direct bond; a substituted or unsubstituted arylene group; or a substituted or unsubstituted divalent heterocyclic group,
Ar20 and Ar21 are the same as or different from each other, and are each independently hydrogen; deuterium; a substituted or unsubstituted aryl group; or a substituted or unsubstituted heterocyclic group,
R201 is hydrogen; deuterium; a halogen group; a substituted or unsubstituted alkyl group; a substituted or unsubstituted cycloalkyl group; a substituted or unsubstituted aryl group; or a substituted or unsubstituted heterocyclic group, and
r201 is an integer from 1 to 8, and when r201 is 2 or higher, two or more R201's are the same as or different from each other.

In an exemplary embodiment of the present specification, L20 and L21 are the same as or different from each other, and are each independently a direct bond; a monocyclic or polycyclic arylene group having 6 to 30 carbon atoms; or a monocyclic or polycyclic divalent heterocyclic group having 2 to 30 carbon atoms.

In an exemplary embodiment of the present specification, L20 and L21 are the same as or different from each other, and are each independently a direct bond; a phenylene group which is unsubstituted or substituted with deuterium; a biphenylylene group which is unsubstituted or substituted with deuterium; a naphthylene group which is unsubstituted or substituted with deuterium; a divalent dibenzofuran group; or a divalent dibenzothiophene group.

In an exemplary embodiment of the present specification, Ar20 is a substituted or unsubstituted heterocyclic group, and Ar21 is a substituted or unsubstituted aryl group.

In an exemplary embodiment of the present specification, Ar20 and Ar21 are the same as or different from each other, and are each independently a substituted or unsubstituted monocyclic or polycyclic aryl group having 6 to 30 carbon atoms; or a substituted or unsubstituted monocyclic or polycyclic heterocyclic group having 2 to 30 carbon atoms.

In an exemplary embodiment of the present specification, Ar20 and Ar21 are the same as or different from each other, and are each independently a substituted or unsubstituted monocyclic to tetracyclic aryl group having 6 to 20 carbon atoms; or a substituted or unsubstituted monocyclic to tetracyclic heterocyclic group having 6 to 20 carbon atoms.

In an exemplary embodiment of the present specification, Ar20 and Ar21 are the same as or different from each other, and are each independently a phenyl group which is unsubstituted or substituted with deuterium or a monocyclic or polycyclic aryl group having 6 to 20 carbon atoms; a biphenyl group which is unsubstituted or substituted with deuterium or a monocyclic or polycyclic aryl group having 6 to 20 carbon atoms; a naphthyl group which is unsubstituted or substituted with a monocyclic or polycyclic aryl group having 6 to 20 carbon atoms; a thiophene group which is unsubstituted or substituted with a monocyclic or polycyclic aryl group having 6 to 30 carbon atoms; a dibenzofuran group which is unsubstituted or substituted with a monocyclic or polycyclic aryl group having 6 to 20 carbon atoms; a naphthobenzofuran group which is unsubstituted or substituted with a monocyclic or polycyclic aryl group having 6 to 20 carbon atoms; a dibenzothiophene group which is unsubstituted or substituted with a monocyclic or polycyclic aryl group having 6 to 20 carbon atoms; or a naphthobenzothiophene group which is unsubstituted or substituted with a monocyclic or polycyclic aryl group having 6 to 20 carbon atoms.

In an exemplary embodiment of the present specification, Ar20 and Ar21 are the same as or different from each other, and are each independently a phenyl group which is unsubstituted or substituted with deuterium; a biphenyl group which is unsubstituted or substituted with deuterium; a terphenyl group; a naphthyl group which is unsubstituted or substituted with deuterium; a thiophene group which is unsubstituted or substituted with a phenyl group; a phenanthrene group; a dibenzofuran group; a naphthobenzofuran group; a dibenzothiophene group; or a naphthobenzothiophene group.

In an exemplary embodiment of the present specification, Ar20 and Ar21 are the same as or different from each other, and are each independently a phenyl group, a 1-naphthyl group, or a 2-naphthyl group.

According to an exemplary embodiment of the present specification, R201 is hydrogen or a naphthyl group.

According to an exemplary embodiment of the present specification, Chemical Formula H-1 may include the following compound.

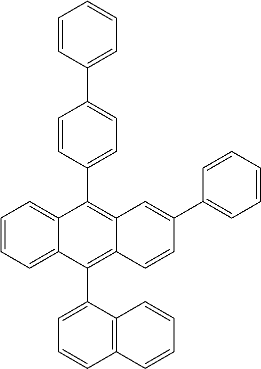 [SMILES: c1ccc(-c2ccc(-c3c4ccccc4c(-c4cccc5ccccc45)c4ccc(-c5ccccc5)cc34)cc2)cc1]

When the light emitting layer emits red light, phosphorescent materials such as bis(1-phenylisoquinoline)acetylacetonate iridium (PIQIr(acac)), bis(1-phenylquinoline)acetylacetonate iridium (PQIr(acac)), tris(1-phenylquinoline)iridium (PQIr) or octaethylporphyrin platinum (PtOEP), or fluorescent materials such as tris(8-hydroxyquinolino)aluminum (Alq₃) may be used as the light emitting dopant, however, the light emitting dopant is not limited thereto. When the light emitting layer emits green light, it is possible to use a phosphorescent material such as fac tris(2-phenylpyridine)iridium (Ir(ppy)₃), or a fluorescent material such as tris(8-hydroxyquinolino)aluminum (Alq₃), as the light emitting dopant, but the light emitting dopant is not limited thereto. When the light emitting layer emits blue light, phosphorescent materials such as (4,6-F2ppy)₂Irpic, or fluorescent materials such as spiro-DPVBi, spiro-6P, distyrylbenzene (DSB), distyrylarylene (DSA), PFO-based polymers or PPV-based polymers may be used as the light emitting dopant, however, the light emitting dopant is not limited thereto.

In an exemplary embodiment of the present specification, the dopant includes a compound of the following Chemical Formula D-1.

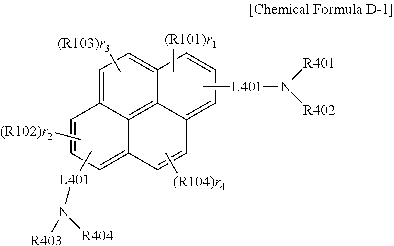

In Chemical Formula D-1,
R101 to R104 are the same as or different from each other, and are each independently hydrogen; deuterium; a substituted or unsubstituted alkyl group; a substituted or unsubstituted aryl group; a substituted or unsubstituted heterocyclic group; or a substituted or unsubstituted silyl group,
L401 and L402 are the same as or different from each other, and are each independently a direct bond; or a substituted or unsubstituted arylene group,
R401 to R404 are the same as or different from each other, and are each independently hydrogen; deuterium; a substituted or unsubstituted alkyl group; a substituted or unsubstituted amine group; a substituted or unsubstituted aryl group; or a substituted or unsubstituted heterocyclic group,
r1 to r4 are an integer from 0 to 2, and
when r1 to r4 are 2 or higher, R101 to R104 in each parenthesis are the same as or different from each other.

In an exemplary embodiment of the present specification, R101 to R104 are the same as or different from each other, and are each independently hydrogen; deuterium; a substituted or unsubstituted alkyl group; or a substituted or unsubstituted silyl group.

In an exemplary embodiment of the present specification, R101 and R102 are the same as or different from each other, and are each independently hydrogen; deuterium; a substituted or unsubstituted alkyl group; or a substituted or unsubstituted silyl group.

In an exemplary embodiment of the present specification, R101 and R102 are the same as or different from each other, and are each independently hydrogen; deuterium; a substituted or unsubstituted alkyl group; or a silyl group that is unsubstituted or substituted with an alkyl group.

In an exemplary embodiment of the present specification, R101 and R102 are the same as or different from each other, and are each independently hydrogen; deuterium; or a silyl group that is unsubstituted or substituted with a methyl group.

In an exemplary embodiment of the present specification, R103 and R104 are the same as or different from each other, and are each independently hydrogen; or deuterium.

In an exemplary embodiment of the present specification, L401 and L402 are each a direct bond.

In an exemplary embodiment of the present specification, R401 to R404 are the same as or different from each other, and are each independently a substituted or unsubstituted aryl group; or a substituted or unsubstituted heterocyclic group.

In an exemplary embodiment of the present specification, R401 to R404 are the same as or different from each other, and are each independently a substituted or unsubstituted monocyclic aryl group; or a substituted or unsubstituted polycyclic aryl group; or a substituted or unsubstituted heterocyclic group.

In an exemplary embodiment of the present specification, R401 to R404 are the same as or different from each other, and are each independently a substituted or unsubstituted phenyl group; a substituted or unsubstituted biphenyl group; a substituted or unsubstituted terphenyl group; a substituted or unsubstituted naphthyl group; a substituted or unsubstituted anthracenyl group; a substituted or unsubstituted phenanthrene group; a substituted or unsubstituted triphenylene group; a substituted or unsubstituted pyrene group; or a substituted or unsubstituted fluorenyl group; a substituted or unsubstituted dibenzofuran group; or a substituted or unsubstituted dibenzothiophene group.

In an exemplary embodiment of the present specification, R401 to R404 are the same as or different from each other, and are each independently a substituted or unsubstituted phenyl group; or a substituted or unsubstituted dibenzofuran group.

In an exemplary embodiment of the present specification, R401 to R404 are the same as or different from each other, and are each independently a phenyl group that is unsubstituted or substituted with one or more of a substituted or unsubstituted alkyl group, a substituted or unsubstituted silyl group, a cyano group; or a substituted or unsubstituted aryl group.

In an exemplary embodiment of the present specification, R401 to R404 are the same as or different from each other, and are each independently a phenyl group that is unsubstituted or substituted with one or more substituents selected from the group consisting of a methyl group, a tert-butyl group, a propyl group, an isopropyl group, a trimethylsilyl group, a cyano group, and a phenyl group that is unsubstituted or substituted with an alkyl group. In an exemplary embodiment of the present specification, Chemical Formula D-1 is represented by the following compound.

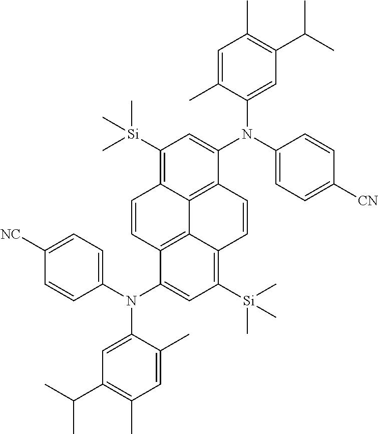 [SMILES: Cc1cc(C)c(N(c2ccc(C#N)cc2)c2cc([Si](C)(C)C)c3ccc4c(N(c5ccc(C#N)cc5)c5cc(C(C)C)c(C)cc5C)cc([Si](C)(C)C)c5ccc2c3c45)cc1C(C)C]

A hole blocking layer may be provided between the electron transport layer and the light emitting layer, and materials known in the art may be used.

The electron transport layer may perform a role of smoothly transporting electrons. An electron transport material is suitably a material having high electron mobility which may proficiently accept electrons from a cathode and transfer the electrons to a light emitting layer. Specific examples thereof include Al complexes of 8-hydroxyquinoline; complexes including Alq₃; organic radical compounds; hydroxyflavon-metal complexes, and the like, but are not limited thereto. The electron transport layer may have a thickness of 1 to 50 nm. When the electron transport layer has a thickness of 1 nm or more, there is an advantage in that it is possible to prevent electron transport characteristics from deteriorating, and when the electron transport layer has a thickness of 50 nm or less, there is an advantage in that it is possible to prevent the driving voltage from being increased in order to improve the movement of electrons due to the too thick electron transport layer.

The electron injection layer may perform a role of smoothly injecting electrons. An electron injection material is preferably a compound which has a capability of transporting electrons, an effect of injecting electrons from a cathode, and an excellent effect of injecting electrons into a light emitting layer or a light emitting material, prevents excitons produced from a light emitting layer from moving to a hole injection layer, and is also excellent in the ability to form a thin film. Specific examples thereof include fluorenone, anthraquinodimethane, diphenoquinone, thiopyran dioxide, oxazole, oxadiazole, triazole, imidazole, perylenetetracarboxylic acid, fluorenylidene methane, anthrone, and the like, and derivatives thereof, metal complex compounds, nitrogen-containing 5-membered ring derivatives, and the like, but are not limited thereto.

The electron injection and transport layer may be manufactured by appropriately selecting materials used for the electron injection layer and the electron transport layer.

The electron injection and transport layer may be manufactured using the compound of Chemical Formula 1.

The electron injection and transport layer may be manufactured using the compound of Chemical Formula 1 and a metal complex together.

The electron injection and transport layer includes the compound of Chemical Formula 1 and the metal complex at a weight ratio of 1:10 to 10:1.

The electron injection and transport layer includes the compound of Chemical Formula 1 and the metal complex at a weight ratio of 1:3 to 3:1.

The metal complex compound includes 8-hydroxyquinolinato lithium, bis(8-hydroxyquinolinato)zinc, bis(8-hydroxyquinolinato)copper, bis(8-hydroxyquinolinato)manganese, tris(8-hydroxyquinolinato)aluminum, tris(2-methyl-8-hydroxyquinolinato)aluminum, tris(8-hydroxyquinolinato)gallium, bis(10-hydroxybenzo[h]quinolinato)beryllium, bis(10-hydroxybenzo[h]quinolinato)zinc, bis(2-methyl-8-quinolinato)chlorogallium, bis(2-methyl-8-quinolinato) (o-cresolato)gallium, bis(2-methyl-8-quinolinato) (1-naphtholato)aluminum, bis(2-methyl-8-quinolinato) (2-naphtholato)gallium and the like, but is not limited thereto.

The hole blocking layer is layer blocking holes from reaching a cathode, and may be generally formed under the same condition as the hole injection layer. Specific examples thereof may include oxadiazole derivatives, triazole derivatives, phenanthroline derivatives, BCP, aluminum complexes and the like, but are not limited thereto.

The organic light emitting device according to the present invention may be a top emission type, a bottom emission type, or a dual emission type according to the material to be used.

The organic light emitting device of the present invention may be manufactured using typical manufacturing methods and materials of an organic light emitting device, except that the above-described compound is used to form an organic material layer having one or more layers.

### PREPARATION EXAMPLES

### Preparation Example 1-1: Preparation of Compound E1

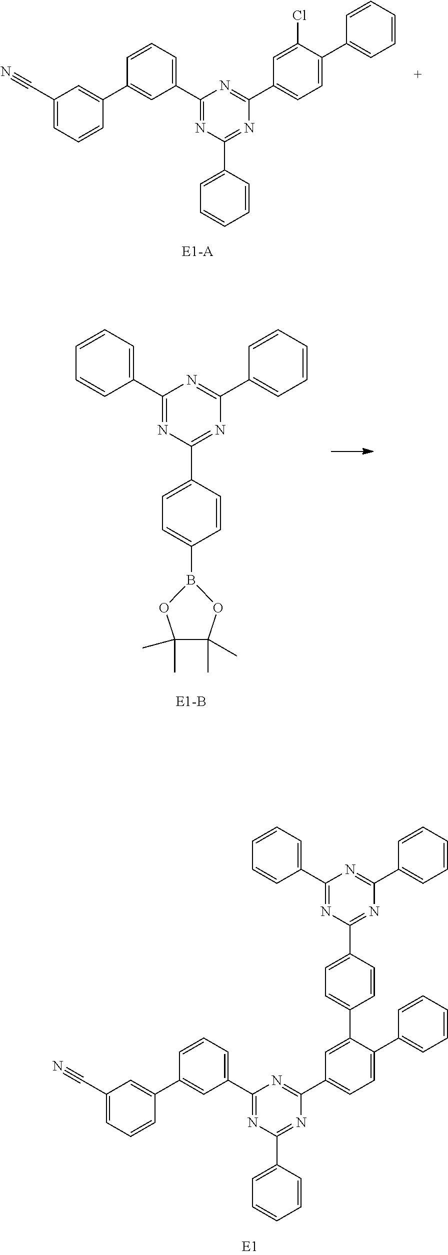 [SMILES: N#Cc1cccc(-c2cccc(-c3nc(-c4ccccc4)nc(-c4ccc(-c5ccccc5)c(Cl)c4)n3)c2)c1 ; CC1(C)OB(c2ccc(-c3nc(-c4ccccc4)nc(-c4ccccc4)n3)cc2)OC1(C)C ; N#Cc1cccc(-c2cccc(-c3nc(-c4ccccc4)nc(-c4ccc(-c5ccccc5)c(-c5ccc(-c6nc(-c7ccccc7)nc(-c7ccccc7)n6)cc5)c4)n3)c2)c1]

E1-A (20 g, 38.4 mmol) and E1-B (16.7 g, 38.4 mmol) were put into 400 ml of 1,4-dioxane in a nitrogen atmosphere, and the resulting mixture was stirred and refluxed. Thereafter, potassium phosphate tribasic (24.4 g, 115.2 mmol) was dissolved in 24 ml of water, the resulting solution was introduced thereinto, the resulting mixture was sufficiently stirred, and then dibenzylideneacetone palladium (0.7 g, 1.2 mmol) and tricyclohexylphosphine (0.6 g, 2.3 mmol) was introduced thereinto. After the reaction for 6 hours, the temperature of the product was lowered to room temperature, and then a produced solid was filtered. The solid was added to and dissolved in 900 mL of chloroform, the solution was washed twice with water, and then the organic layer was separated, anhydrous magnesium sulfate was put thereinto, and the resulting mixture was stirred, and then filtered, and the filtrate was distilled under reduced pressure. The concentrated compound was recrystallized with chloroform and ethyl acetate to prepare E1 (4.6 g, 15%, MS: [M+H]+=794) which was a yellow solid compound.

### Preparation Example 1-2: Preparation of Compound E2

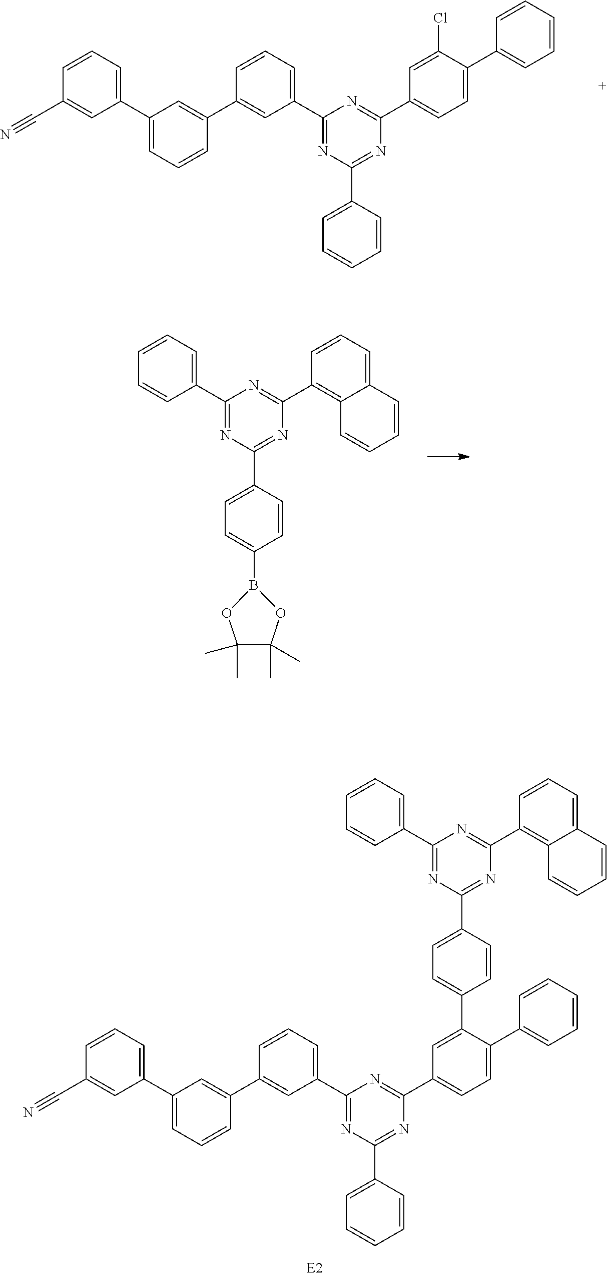 [SMILES: N#Cc1cccc(-c2cccc(-c3cccc(-c4nc(-c5ccccc5)nc(-c5ccc(-c6ccccc6)c(Cl)c5)n4)c3)c2)c1 ; CC1(C)OB(c2ccc(-c3nc(-c4ccccc4)nc(-c4cccc5ccccc45)n3)cc2)OC1(C)C ; N#Cc1cccc(-c2cccc(-c3cccc(-c4nc(-c5ccccc5)nc(-c5ccc(-c6ccccc6)c(-c6ccc(-c7nc(-c8ccccc8)nc(-c8cccc9ccccc89)n7)cc6)c5)n4)c3)c2)c1]

Compound E2 was prepared in the same manner as in the preparation method in Preparation Example 1-1, except that each starting material was used as in the reaction scheme.

MS: [M+H]+=920

### Preparation Example 1-3: Preparation of Compound E3

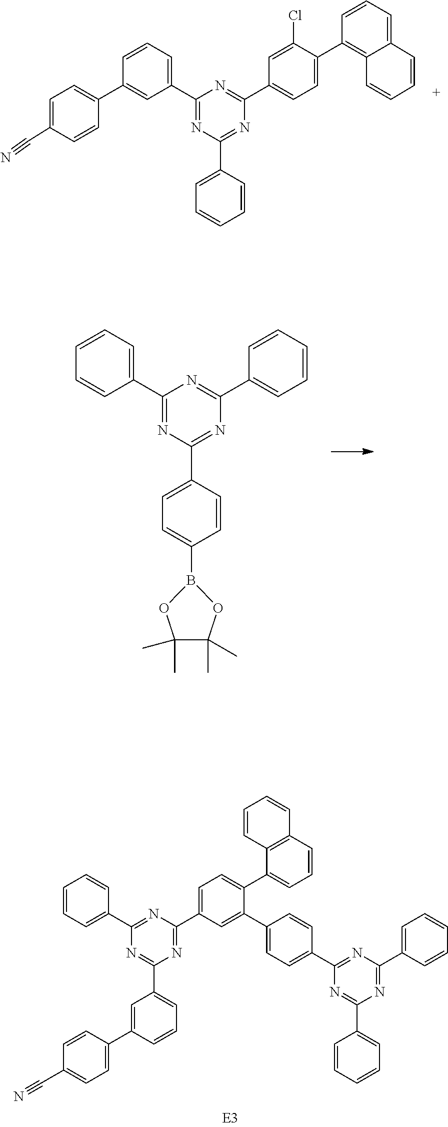 [SMILES: N#Cc1ccc(-c2cccc(-c3nc(-c4ccccc4)nc(-c4ccc(-c5cccc6ccccc56)c(Cl)c4)n3)c2)cc1 ; CC1(C)OB(c2ccc(-c3nc(-c4ccccc4)nc(-c4ccccc4)n3)cc2)OC1(C)C ; N#Cc1ccc(-c2cccc(-c3nc(-c4ccccc4)nc(-c4ccc(-c5cccc6ccccc56)c(-c5ccc(-c6nc(-c7ccccc7)nc(-c7ccccc7)n6)cc5)c4)n3)c2)cc1]

Compound E3 was prepared in the same manner as in the preparation method in Preparation Example 1-1, except that each starting material was used as in the reaction scheme.

MS: [M+H]+=844

### Preparation Example 1-4: Preparation of Compound E4

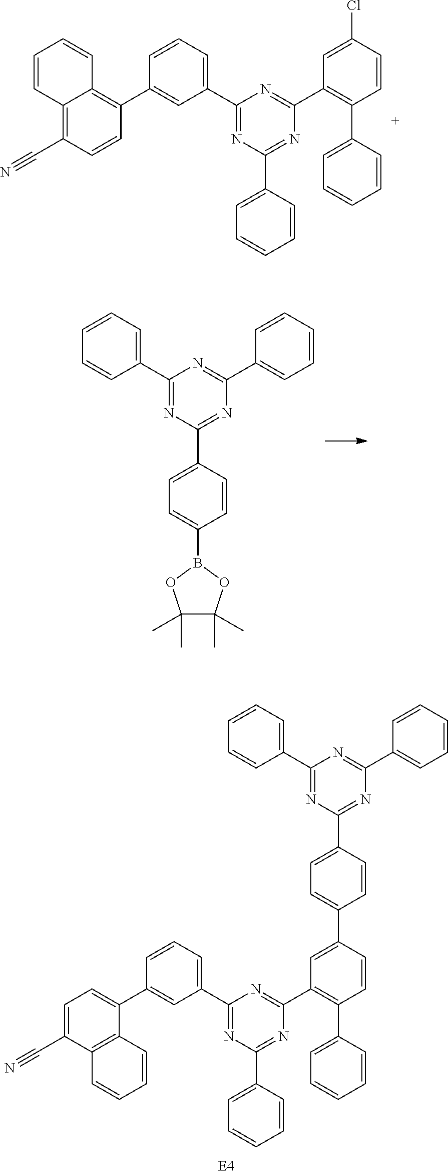 [SMILES: N#Cc1ccc(-c2cccc(-c3nc(-c4ccccc4)nc(-c4cc(Cl)ccc4-c4ccccc4)n3)c2)c2ccccc12 ; CC1(C)OB(c2ccc(-c3nc(-c4ccccc4)nc(-c4ccccc4)n3)cc2)OC1(C)C ; N#Cc1ccc(-c2cccc(-c3nc(-c4ccccc4)nc(-c4cc(-c5ccc(-c6nc(-c7ccccc7)nc(-c7ccccc7)n6)cc5)ccc4-c4ccccc4)n3)c2)c2ccccc12]

Compound E4 was prepared in the same manner as in the preparation method in Preparation Example 1-1, except that each starting material was used as in the reaction scheme.

MS: [M+H]+=844

### Preparation Example 1-5: Preparation of Compound E5

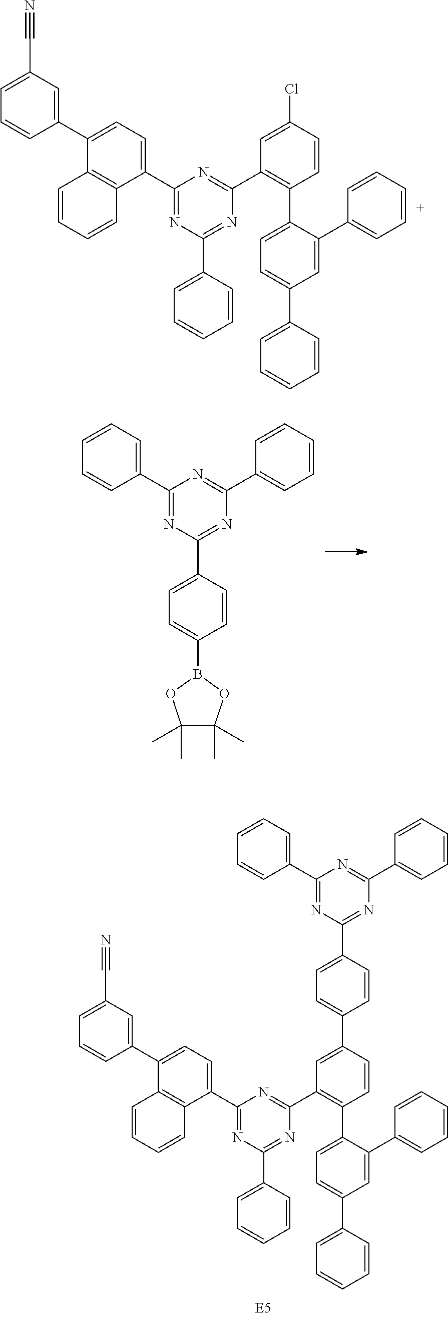

Compound E5 was prepared in the same manner as in the preparation method in Preparation Example 1-1, except that each starting material was used as in the reaction scheme.

MS: [M+H]+=996

### Preparation Example 1-6: Preparation of Compound E6

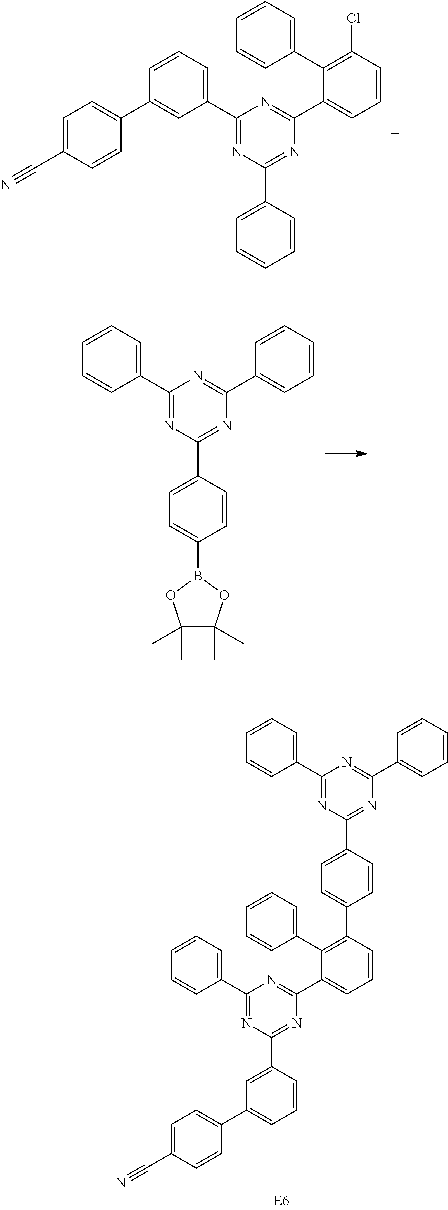 [SMILES: N#Cc1ccc(-c2cccc(-c3nc(-c4ccccc4)nc(-c4cccc(Cl)c4-c4ccccc4)n3)c2)cc1 ; CC1(C)OB(c2ccc(-c3nc(-c4ccccc4)nc(-c4ccccc4)n3)cc2)OC1(C)C ; N#Cc1ccc(-c2cccc(-c3nc(-c4ccccc4)nc(-c4cccc(-c5ccc(-c6nc(-c7ccccc7)nc(-c7ccccc7)n6)cc5)c4-c4ccccc4)n3)c2)cc1]

Compound E6 was prepared in the same manner as in the preparation method in Preparation Example 1-1, except that each starting material was used as in the reaction scheme.

MS: [M+H]+=794

### Preparation Example 1-7: Preparation of Compound E7

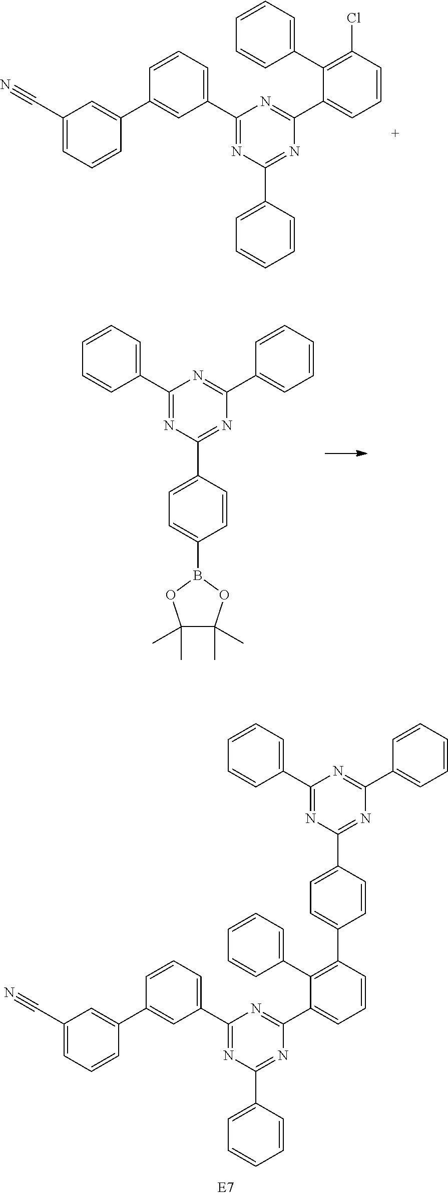 [SMILES: N#Cc1cccc(-c2cccc(-c3nc(-c4ccccc4)nc(-c4cccc(Cl)c4-c4ccccc4)n3)c2)c1 ; CC1(C)OB(c2ccc(-c3nc(-c4ccccc4)nc(-c4ccccc4)n3)cc2)OC1(C)C ; N#Cc1cccc(-c2cccc(-c3nc(-c4ccccc4)nc(-c4cccc(-c5ccc(-c6nc(-c7ccccc7)nc(-c7ccccc7)n6)cc5)c4-c4ccccc4)n3)c2)c1]

Compound E7 was prepared in the same manner as in the preparation method in Preparation Example 1-1, except that each starting material was used as in the reaction scheme.

MS: [M+H]+=794

### Preparation Example 1-8: Preparation of Compound E8

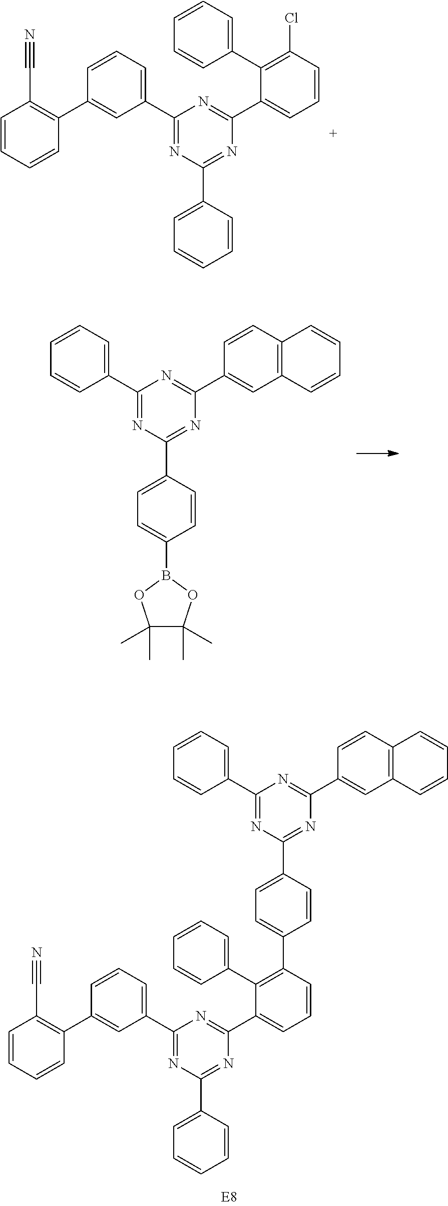 [SMILES: N#Cc1ccccc1-c1cccc(-c2nc(-c3ccccc3)nc(-c3cccc(Cl)c3-c3ccccc3)n2)c1 ; CC1(C)OB(c2ccc(-c3nc(-c4ccccc4)nc(-c4ccc5ccccc5c4)n3)cc2)OC1(C)C ; N#Cc1ccccc1-c1cccc(-c2nc(-c3ccccc3)nc(-c3cccc(-c4ccc(-c5nc(-c6ccccc6)nc(-c6ccc7ccccc7c6)n5)cc4)c3-c3ccccc3)n2)c1]

Compound E8 was prepared in the same manner as in the preparation method in Preparation Example 1-1, except that each starting material was used as in the reaction scheme.

MS: [M+H]+=844

### Preparation Example 1-9: Preparation of Compound E9

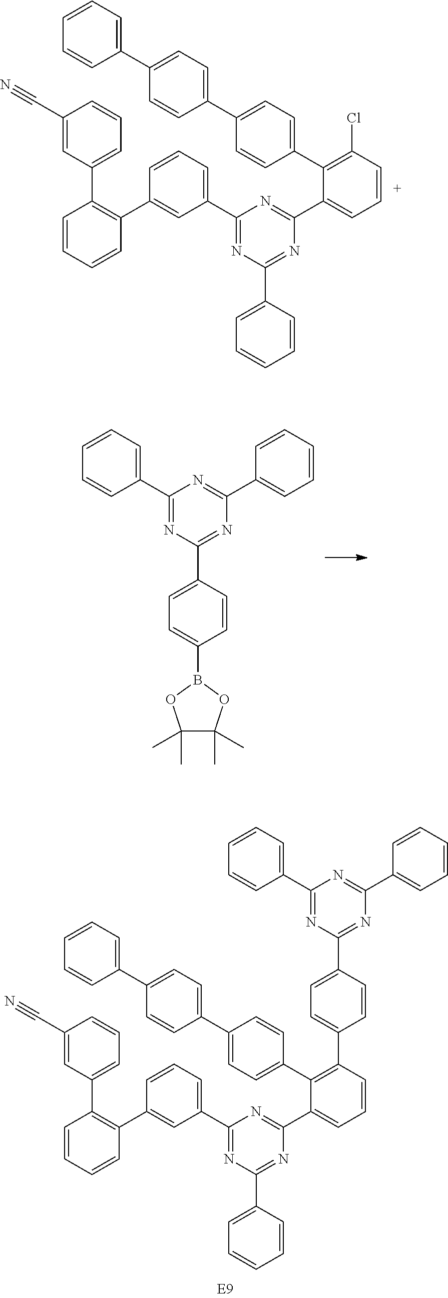 [SMILES: N#Cc1cccc(-c2ccccc2-c2cccc(-c3nc(-c4ccccc4)nc(-c4cccc(Cl)c4-c4ccc(-c5ccc(-c6ccccc6)cc5)cc4)n3)c2)c1 ; CC1(C)OB(c2ccc(-c3nc(-c4ccccc4)nc(-c4ccccc4)n3)cc2)OC1(C)C ; N#Cc1cccc(-c2ccccc2-c2cccc(-c3nc(-c4ccccc4)nc(-c4cccc(-c5ccc(-c6nc(-c7ccccc7)nc(-c7ccccc7)n6)cc5)c4-c4ccc(-c5ccc(-c6ccccc6)cc5)cc4)n3)c2)c1]

Compound E9 was prepared in the same manner as in the preparation method in Preparation Example 1-1, except that each starting material was used as in the reaction scheme.

MS: [M+H]+=1022

### Preparation Example 1-10: Preparation of Compound E10

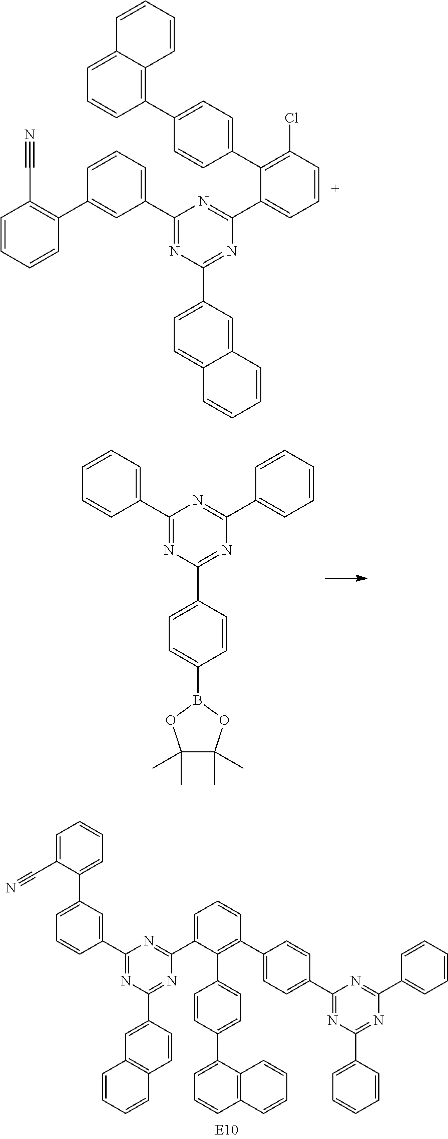 [SMILES: N#Cc1ccccc1-c1cccc(-c2nc(-c3ccc4ccccc4c3)nc(-c3cccc(Cl)c3-c3ccc(-c4cccc5ccccc45)cc3)n2)c1 ; CC1(C)OB(c2ccc(-c3nc(-c4ccccc4)nc(-c4ccccc4)n3)cc2)OC1(C)C ; N#Cc1ccccc1-c1cccc(-c2nc(-c3ccc4ccccc4c3)nc(-c3cccc(-c4ccc(-c5nc(-c6ccccc6)nc(-c6ccccc6)n5)cc4)c3-c3ccc(-c4cccc5ccccc45)cc3)n2)c1]

Compound E10 was prepared in the same manner as in the preparation method in Preparation Example 1-1, except that each starting material was used as in the reaction scheme.

MS: [M+H]+=970

### Preparation Example 1-11: Preparation of Compound E11

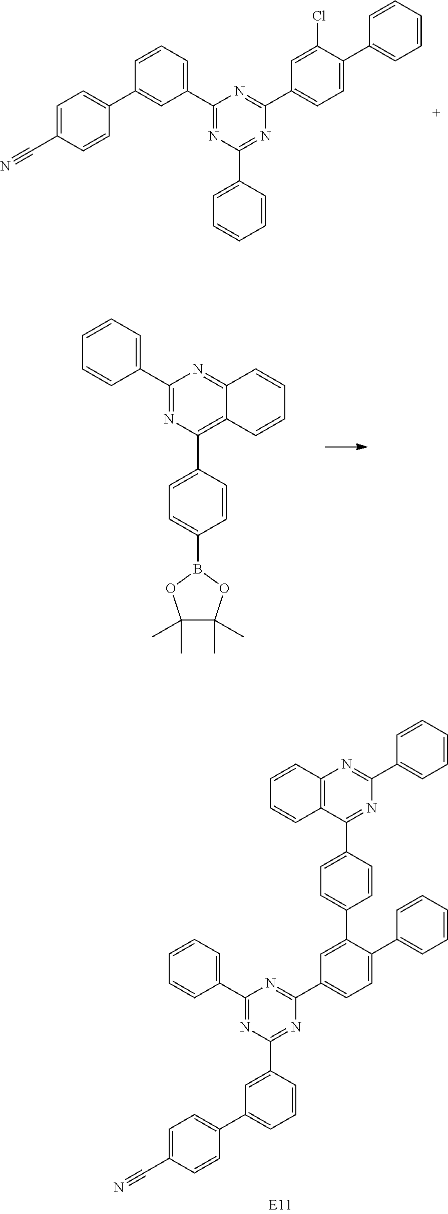 [SMILES: N#Cc1ccc(-c2cccc(-c3nc(-c4ccccc4)nc(-c4ccc(-c5ccccc5)c(Cl)c4)n3)c2)cc1 ; CC1(C)OB(c2ccc(-c3nc(-c4ccccc4)nc4ccccc34)cc2)OC1(C)C ; N#Cc1ccc(-c2cccc(-c3nc(-c4ccccc4)nc(-c4ccc(-c5ccccc5)c(-c5ccc(-c6nc(-c7ccccc7)nc7ccccc67)cc5)c4)n3)c2)cc1]

Compound E11 was prepared in the same manner as in the preparation method in Preparation Example 1-1, except that each starting material was used as in the reaction scheme.

MS: [M+H]+=767

### Preparation Example 1-12: Preparation of Compound E12

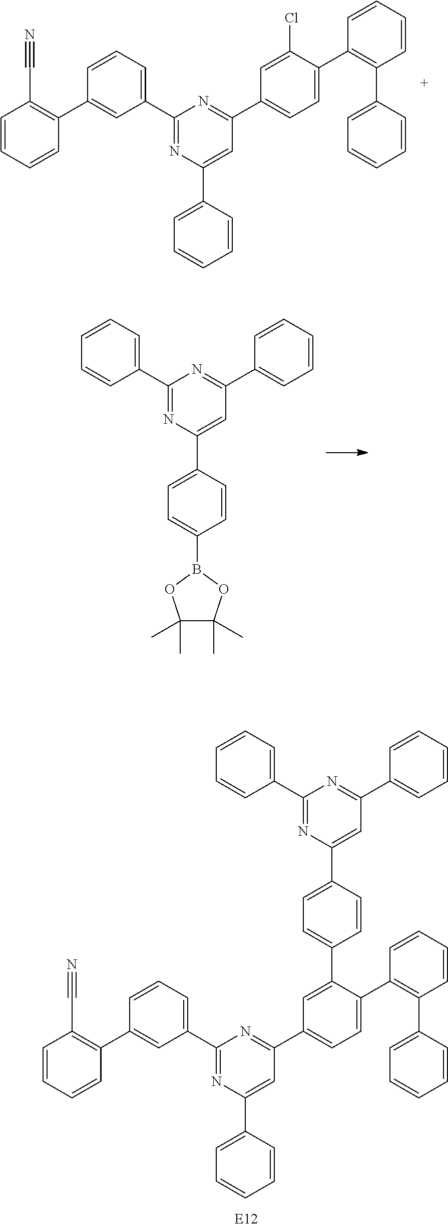 [SMILES: N#Cc1ccccc1-c1cccc(-c2nc(-c3ccccc3)cc(-c3ccc(-c4ccccc4-c4ccccc4)c(Cl)c3)n2)c1 ; CC1(C)OB(c2ccc(-c3cc(-c4ccccc4)nc(-c4ccccc4)n3)cc2)OC1(C)C ; N#Cc1ccccc1-c1cccc(-c2nc(-c3ccccc3)cc(-c3ccc(-c4ccccc4-c4ccccc4)c(-c4ccc(-c5cc(-c6ccccc6)nc(-c6ccccc6)n5)cc4)c3)n2)c1]

Compound E12 was prepared in the same manner as in the preparation method in Preparation Example 1-1, except that each starting material was used as in the reaction scheme.

MS: [M+H]+=868

### Preparation Example 1-13: Preparation of Compound E13

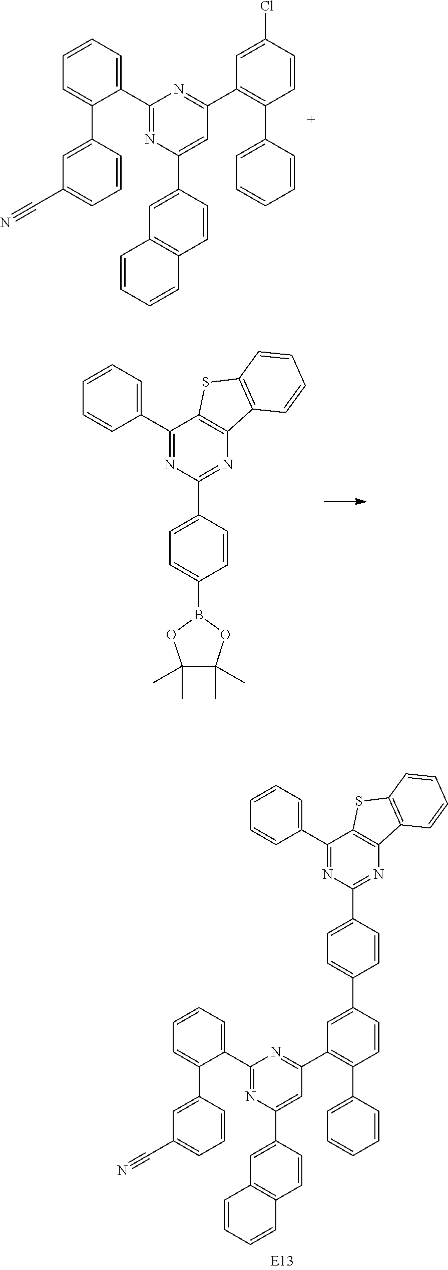 [SMILES: N#Cc1cccc(-c2ccccc2-c2nc(-c3ccc4ccccc4c3)cc(-c3cc(Cl)ccc3-c3ccccc3)n2)c1 ; CC1(C)OB(c2ccc(-c3nc(-c4ccccc4)c4sc5ccccc5c4n3)cc2)OC1(C)C ; N#Cc1cccc(-c2ccccc2-c2nc(-c3ccc4ccccc4c3)cc(-c3cc(-c4ccc(-c5nc(-c6ccccc6)c6sc7ccccc7c6n5)cc4)ccc3-c3ccccc3)n2)c1]

Compound E13 was prepared in the same manner as in the preparation method in Preparation Example 1-1, except that each starting material was used as in the reaction scheme.

MS: [M+H]+=872

### Preparation Example 1-14: Preparation of Compound E14

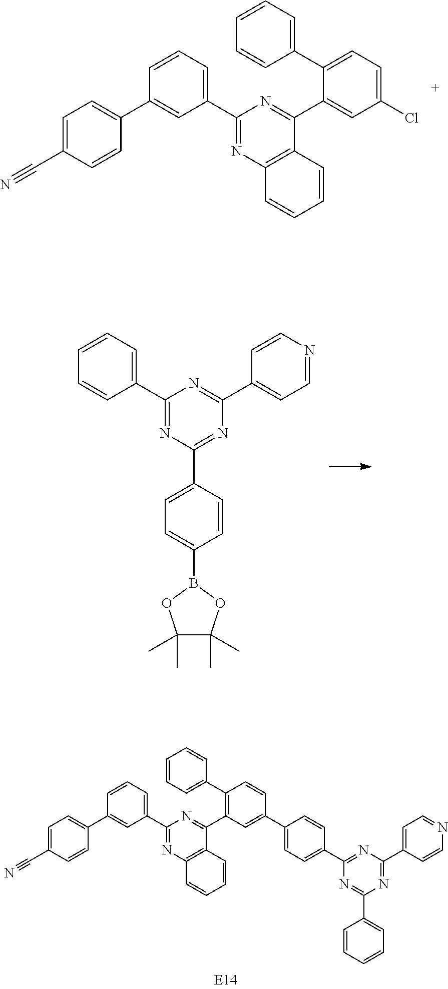 [SMILES: N#Cc1ccc(-c2cccc(-c3nc(-c4cc(Cl)ccc4-c4ccccc4)c4ccccc4n3)c2)cc1 ; CC1(C)OB(c2ccc(-c3nc(-c4ccccc4)nc(-c4ccncc4)n3)cc2)OC1(C)C ; N#Cc1ccc(-c2cccc(-c3nc(-c4cc(-c5ccc(-c6nc(-c7ccccc7)nc(-c7ccncc7)n6)cc5)ccc4-c4ccccc4)c4ccccc4n3)c2)cc1]

Compound E14 was prepared in the same manner as in the preparation method in Preparation Example 1-1, except that each starting material was used as in the reaction scheme.

MS: [M+H]+=768

### Preparation Example 1-15: Preparation of Compound E15

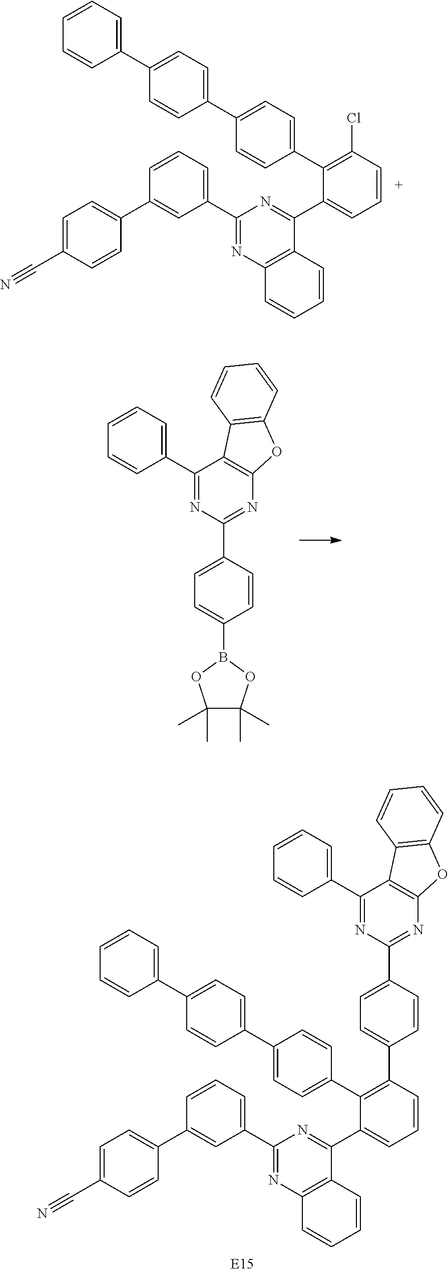 [SMILES: N#Cc1ccc(-c2cccc(-c3nc(-c4cccc(Cl)c4-c4ccc(-c5ccc(-c6ccccc6)cc5)cc4)c4ccccc4n3)c2)cc1 ; CC1(C)OB(c2ccc(-c3nc(-c4ccccc4)c4c(n3)oc3ccccc34)cc2)OC1(C)C ; N#Cc1ccc(-c2cccc(-c3nc(-c4cccc(-c5ccc(-c6nc(-c7ccccc7)c7c(n6)oc6ccccc67)cc5)c4-c4ccc(-c5ccc(-c6ccccc6)cc5)cc4)c4ccccc4n3)c2)cc1]

Compound E15 was prepared in the same manner as in the preparation method in Preparation Example 1-1, except that each starting material was used as in the reaction scheme.

MS: [M+H]+=932

### Preparation Example 1-16: Preparation of Compound E16

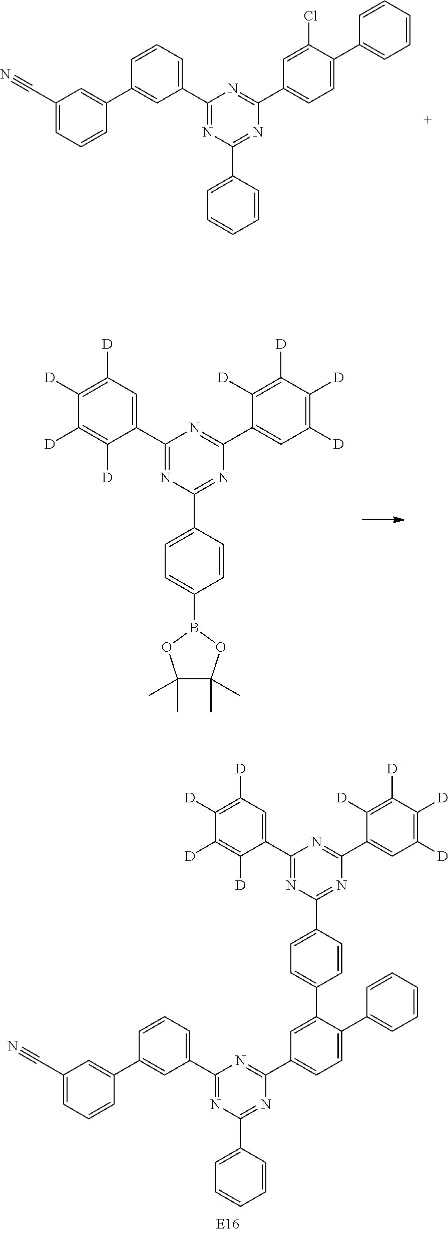 [SMILES: N#Cc1cccc(-c2cccc(-c3nc(-c4ccccc4)nc(-c4ccc(-c5ccccc5)c(Cl)c4)n3)c2)c1 ; [2H]c1cc(-c2nc(-c3ccc(B4OC(C)(C)C(C)(C)O4)cc3)nc(-c3cc([2H])c([2H])c([2H])c3[2H])n2)c([2H])c([2H])c1[2H] ; [2H]c1cc(-c2nc(-c3ccc(-c4cc(-c5nc(-c6ccccc6)nc(-c6cccc(-c7cccc(C#N)c7)c6)n5)ccc4-c4ccccc4)cc3)nc(-c3cc([2H])c([2H])c([2H])c3[2H])n2)c([2H])c([2H])c1[2H]]

Compound E16 was prepared in the same manner as in the preparation method in Preparation Example 1-1, except that each starting material was used as in the reaction scheme.

MS: [M+H]+=802

### Preparation Example 1-17: Preparation of Compound E17

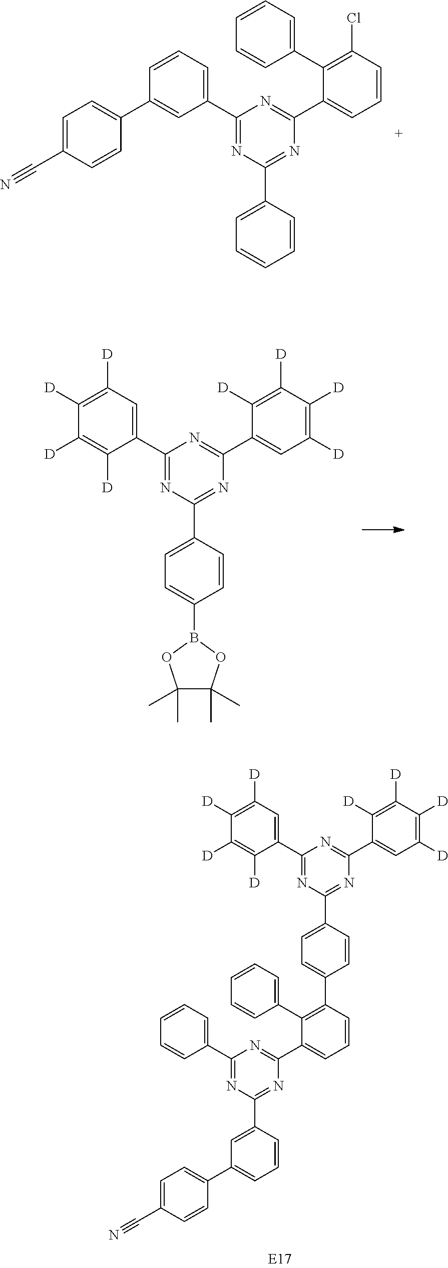 [SMILES: N#Cc1ccc(-c2cccc(-c3nc(-c4ccccc4)nc(-c4cccc(Cl)c4-c4ccccc4)n3)c2)cc1 ; [2H]c1cc(-c2nc(-c3ccc(B4OC(C)(C)C(C)(C)O4)cc3)nc(-c3cc([2H])c([2H])c([2H])c3[2H])n2)c([2H])c([2H])c1[2H] ; [2H]c1cc(-c2nc(-c3ccc(-c4cccc(-c5nc(-c6ccccc6)nc(-c6cccc(-c7ccc(C#N)cc7)c6)n5)c4-c4ccccc4)cc3)nc(-c3cc([2H])c([2H])c([2H])c3[2H])n2)c([2H])c([2H])c1[2H]]

Compound E17 was prepared in the same manner as in the preparation method in Preparation Example 1-1, except that each starting material was used as in the reaction scheme.

MS: [M+H]+=802

### EXAMPLES

### Example 1-1

A glass substrate thinly coated with indium tin oxide (ITO) to have a thickness of 1000 Å was put into distilled water in which a detergent was dissolved, and ultrasonically washed. In this case, a product manufactured by the Fischer Co., was used as the detergent, and distilled water twice filtered using a filter manufactured by Millipore Co., was used as the distilled water. After the ITO was washed for 30 minutes, ultrasonic washing was repeated twice by using distilled water for 10 minutes. After the washing using distilled water was completed, ultrasonic washing was conducted by using isopropyl alcohol, acetone, and methanol solvents, and the resulting product was dried and then transported to a plasma washing machine. Furthermore, the substrate was cleaned by using oxygen plasma for 5 minutes, and then was transported to a vacuum deposition machine.

The following Compound HI-A was thermally vacuum deposited to have a thickness of 600 Å on the transparent ITO electrode, which was thus prepared, thereby forming a hole injection layer. The following compound HAT and the following compound HT-A were sequentially vacuum-deposited to have a thickness of 50 Å and 60 Å, respectively, on the hole injection layer, thereby forming a first hole transport layer and a second hole transport layer.

Subsequently, the following compound BH and Compound BD were vacuum-deposited at a weight ratio of 25:1 to have a film thickness of 200 Å on the second hole transport layer, thereby forming a light emitting layer.

Compound E1 previously prepared and the following compound LiQ were vacuum-deposited at a weight ratio of 1:1 on the light emitting layer, thereby forming an electron injection and transport layer having a thickness of 350 Å. Lithium fluoride (LiF) and aluminum were sequentially deposited to have a thickness of 10 Å and 1000 Å, respectively, on the electron injection and transport layer, thereby forming a cathode.

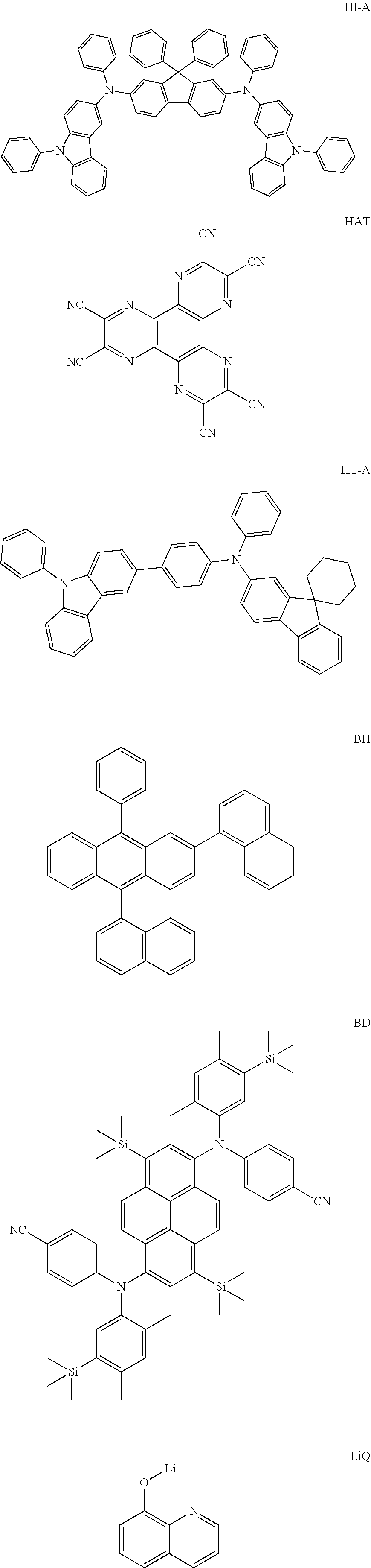

In the aforementioned procedure, the deposition rate of the organic material was maintained at 0.4 Å/sec to 0.9 Å/sec, the deposition rates of lithium fluoride and aluminum of the negative electrode were maintained at 0.3 Å/sec and at 2 Å/sec, respectively, and the degree of vacuum during the deposition was maintained at 1×10⁻⁷ to 5×10⁻⁵ torr, thereby manufacturing an organic light emitting device.

### Examples 1-2 to 1-17

Organic light emitting devices were manufactured in the same manner as in Example 1-1, except that Compounds E2 to E17 described in the following Table 1 were each used instead of Compound E1 in Example 1-1.

### Comparative Examples 1-1 to 1-11

Organic light emitting devices were manufactured in the same manner as in Example 1-1, except that Compounds ET-1 to ET-11 in the following Table 1 were each used instead of Compound E1 in Example 1-1. The structures of Compounds ET-1 to ET-11 in the following Table 1 are as follows.

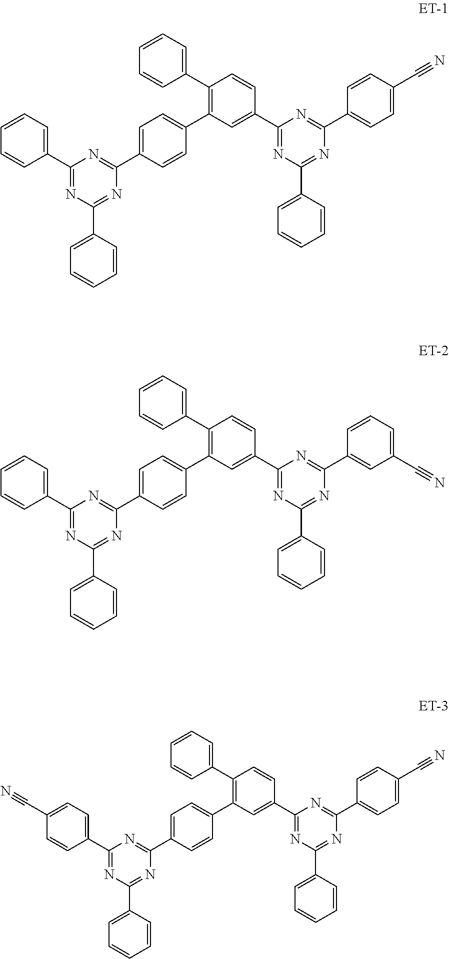 [SMILES: N#Cc1ccc(-c2nc(-c3ccccc3)nc(-c3ccc(-c4ccccc4)c(-c4ccc(-c5nc(-c6ccccc6)nc(-c6ccccc6)n5)cc4)c3)n2)cc1 ; N#Cc1cccc(-c2nc(-c3ccccc3)nc(-c3ccc(-c4ccccc4)c(-c4ccc(-c5nc(-c6ccccc6)nc(-c6ccccc6)n5)cc4)c3)n2)c1 ; N#Cc1ccc(-c2nc(-c3ccccc3)nc(-c3ccc(-c4cc(-c5nc(-c6ccccc6)nc(-c6ccc(C#N)cc6)n5)ccc4-c4ccccc4)cc3)n2)cc1]

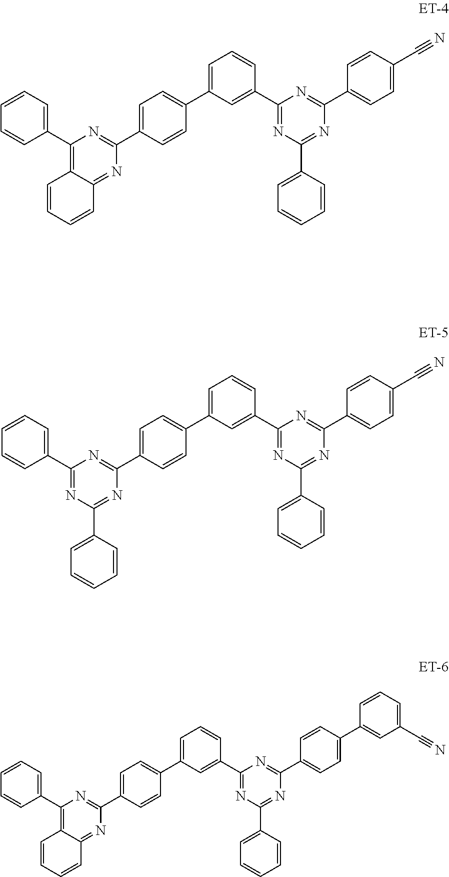 [SMILES: N#Cc1ccc(-c2nc(-c3ccccc3)nc(-c3cccc(-c4ccc(-c5nc(-c6ccccc6)c6ccccc6n5)cc4)c3)n2)cc1 ; N#Cc1ccc(-c2nc(-c3ccccc3)nc(-c3cccc(-c4ccc(-c5nc(-c6ccccc6)nc(-c6ccccc6)n5)cc4)c3)n2)cc1 ; N#Cc1cccc(-c2ccc(-c3nc(-c4ccccc4)nc(-c4cccc(-c5ccc(-c6nc(-c7ccccc7)c7ccccc7n6)cc5)c4)n3)cc2)c1]

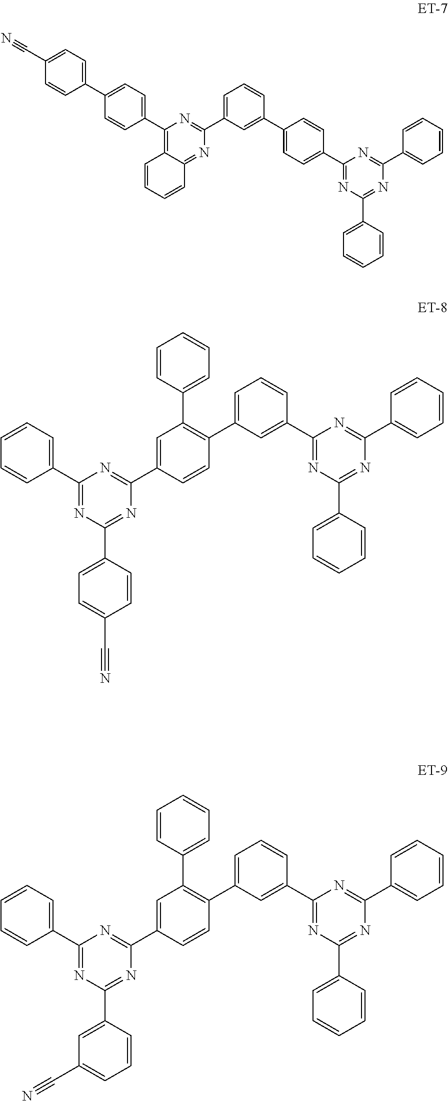 [SMILES: N#Cc1ccc(-c2ccc(-c3nc(-c4cccc(-c5ccc(-c6nc(-c7ccccc7)nc(-c7ccccc7)n6)cc5)c4)nc4ccccc34)cc2)cc1 ; N#Cc1ccc(-c2nc(-c3ccccc3)nc(-c3ccc(-c4cccc(-c5nc(-c6ccccc6)nc(-c6ccccc6)n5)c4)c(-c4ccccc4)c3)n2)cc1 ; N#Cc1cccc(-c2nc(-c3ccccc3)nc(-c3ccc(-c4cccc(-c5nc(-c6ccccc6)nc(-c6ccccc6)n5)c4)c(-c4ccccc4)c3)n2)c1]

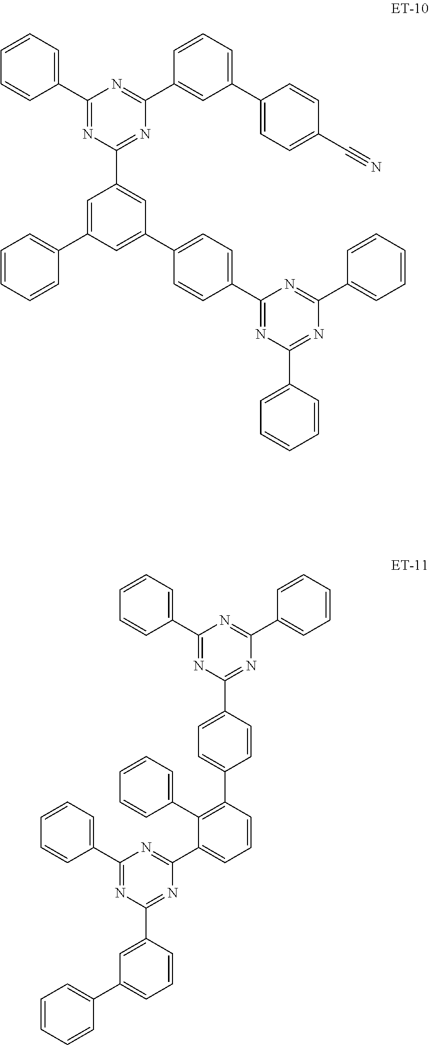 [SMILES: N#Cc1ccc(-c2cccc(-c3nc(-c4ccccc4)nc(-c4cc(-c5ccccc5)cc(-c5ccc(-c6nc(-c7ccccc7)nc(-c7ccccc7)n6)cc5)c4)n3)c2)cc1 ; c1ccc(-c2cccc(-c3nc(-c4ccccc4)nc(-c4cccc(-c5ccc(-c6nc(-c7ccccc7)nc(-c7ccccc7)n6)cc5)c4-c4ccccc4)n3)c2)cc1]

### Experimental Examples

For the organic light emitting devices manufactured in Examples 1-1 to 1-17 and Comparative Examples 1-1 to 1-11, the driving voltage and the light emitting efficiency were measured at a current density of 10 mA/cm², and a time (T90) for reaching a 90% value compared to the initial luminance was measured at a current density of 20 mA/cm². The results are shown in the following Table 1.

TABLE 1

| | | Voltage | Efficiency | | Service life |
|---|---|---|---|---|---|
| | | (V) | (cd/A) | Color | (hr) |
| | | (@10 | (@10 | coordinate | (T90 at 20 |
| Classification | Compound | mA/cm²) | mA/cm²) | (x, y) | mA/cm²) |
| Example 1-1 | E1 | 4.20 | 4.80 | (0.138, 0.113) | 230 |
| Example 1-2 | E2 | 4.24 | 4.75 | (0.138, 0.112) | 244 |
| Example 1-3 | E3 | 4.22 | 4.78 | (0.138, 0.112) | 242 |
| Example 1-4 | E4 | 4.29 | 4.68 | (0.138, 0.113) | 258 |
| Example 1-5 | E5 | 4.33 | 4.61 | (0.138, 0.113) | 237 |
| Example 1-6 | E6 | 4.16 | 4.78 | (0.138, 0.113) | 253 |
| Example 1-7 | E7 | 4.20 | 4.73 | (0.138, 0.112) | 268 |
| Example 1-8 | E8 | 4.17 | 4.77 | (0.138, 0.113) | 258 |
| Example 1-9 | E9 | 4.25 | 4.61 | (0.138, 0.113) | 248 |
| Example 1-10 | E10 | 4.25 | 4.69 | (0.138, 0.112) | 228 |
| Example 1-11 | E11 | 4.24 | 4.73 | (0.138, 0.112) | 228 |
| Example 1-12 | E12 | 4.41 | 4.50 | (0.138, 0.113) | 205 |
| Example 1-13 | E13 | 4.41 | 4.49 | (0.138, 0.113) | 207 |
| Example 1-14 | E14 | 4.30 | 4.56 | (0.138, 0.112) | 209 |
| Example 1-15 | E15 | 4.43 | 4.33 | (0.138, 0.112) | 216 |
| Example 1-16 | E16 | 4.20 | 4.80 | (0.138, 0.113) | 244 |
| Example 1-17 | E17 | 4.16 | 4.78 | (0.138, 0.113) | 271 |
| Comparative | ET-1 | 5.32 | 2.17 | (0.138, 0.113) | 82 |
| Example 1-1 | | | | | |
| Comparative | ET-2 | 5.36 | 2.25 | (0.138, 0.112) | 86 |
| Example 1-2 | | | | | |
| Comparative | ET-3 | 6.20 | 1.30 | (0.138, 0.113) | 20 |
| Example 1-3 | | | | | |
| Comparative | ET-4 | 5.40 | 1.73 | (0.138, 0.113) | 61 |
| Example 1-4 | | | | | |
| Comparative | ET-5 | 5.58 | 1.65 | (0.138, 0.112) | 51 |
| Example 1-5 | | | | | |
| Comparative | ET-6 | 4.65 | 3.47 | (0.138, 0.113) | 164 |
| Example 1-6 | | | | | |
| Comparative | ET-7 | 4.87 | 2.38 | (0.138, 0.112) | 123 |
| Example 1-7 | | | | | |
| Comparative | ET-8 | 4.83 | 2.38 | (0.138, 0.113) | 123 |
| Example 1-8 | | | | | |
| Comparative | ET-9 | 4.78 | 2.51 | (0.138, 0.113) | 119 |
| Example 1-9 | | | | | |
| Comparative | ET-10 | 4.56 | 3.99 | (0.138, 0.113) | 180 |
| Example 1-10 | | | | | |
| Comparative | ET-11 | 4.39 | 3.42 | (0.138, 0.114) | 45 |
| Example 1-11 | | | | | |

As shown in Table 1 above, the organic light emitting devices of Examples 1-1 to 1-17 in which the compound represented by Chemical Formula 1 according to the present specification was used in the electron injection and electron transport layers of the organic light emitting devices exhibited low driving voltage and excellent characteristics in terms of efficiency and/or service life, compared to Comparative Examples 1-1 to 1-11.

Specifically, Compounds ET-1 to ET-5 are different from the compound of Chemical Formula 1 of the present application in that Compounds ET-1 to ET-5 do not have a linker corresponding to L in Chemical Formula 1 of the present application, Compounds ET-6 and ET-7 are different from the compound of Chemical Formula 1 of the present application in that Compounds ET-6 and ET-7 do not have an aryl group corresponding to Ar21 to Ar23 of Chemical Formula 1 of the present application, Compounds ET-8 and ET-9 are different from the compound of Chemical Formula 1 of the present application in that Compounds ET-8 and ET-9 do not have an aryl group substituted with a cyano group corresponding to Ar1 of Chemical Formula 1 of the present application, Compound ET-10 is different from the compound of Chemical Formula 1 of the present application in that Compound ET-10 is different from the aryl group of Ar21 to Ar23 of Chemical Formula 1 of the present application in terms of the position of the substituent, and Compound ET-11 is different from the compound of Chemical Formula 1 of the present application in that Compounds ET-11 does not have —CN (a cyano group).

As described above, Comparative Examples 1-1 to 1-11 using Compounds ET-1 to ET-11 had reduced electron mobility due to an increase in the energy barrier with the light emitting layer, and thus exhibited higher driving voltage, lower efficiency and/or lower service life characteristics than Examples 1-1 to 1-17 using the compound of Chemical Formula 1 of the present application.

What is claimed is:

1. A compound of the following Chemical Formula 1: 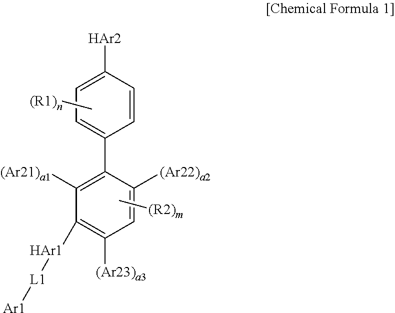
wherein, in Chemical Formula 1,
HAr1 is a divalent triazinyl group that is unsubstituted or substituted with one substituent selected from the group consisting of deuterium, a substituted or unsubstituted aryl group having 6 to 60 carbon atoms, and a substituted or unsubstituted heteroaryl group having 2 to 60 carbon atoms; a divalent pyrimidinyl group that is unsubstituted or substituted with one or two substituents each independently selected from the group consisting of deuterium, a substituted or unsubstituted alkyl group having 1 to 60 carbon atoms, a substituted or unsubstituted aryl group having 6 to 60 carbon atoms, and a substituted or unsubstituted heteroaryl group having 2 to 60 carbon atoms; a divalent quinazolinyl group that is unsubstituted or substituted with deuterium; a divalent benzothienopyrimidinyl group that is unsubstituted or substituted with one substituent selected from the group consisting of deuterium, a substituted or unsubstituted aryl group having 6 to 60 carbon atoms, and a substituted or unsubstituted heteroaryl group having 2 to 60 carbon atoms; or a divalent benzofuropyrimidinyl group that is unsubstituted or substituted with one substituent selected from the group consisting of deuterium, a substituted or unsubstituted aryl group having 6 to 60 carbon atoms, and a substituted or unsubstituted heteroaryl group having 2 to 60 carbon atoms,
HAr2 is a triazinyl group that is unsubstituted or substituted with two substituents each independently selected from the group consisting of deuterium, a substituted or unsubstituted aryl group having 6 to 60 carbon atoms, and a substituted or unsubstituted heteroaryl group having 2 to 60 carbon atoms; a pyrimidinyl group that is unsubstituted or substituted with one to three substituents each independently selected from the group consisting of deuterium, a substituted or unsubstituted alkyl group having 1 to 60 carbon atoms, a substituted or unsubstituted aryl group having 6 to 60 carbon atoms, and a substituted or unsubstituted heteroaryl group having 2 to 60 carbon atoms; a quinazolinyl group that is unsubstituted or substituted with one substituent selected from the group consisting of deuterium, a substituted or unsubstituted aryl group having 6 to 60 carbon atoms, and a substituted or unsubstituted heteroaryl group having 2 to 60 carbon atoms; a benzothienopyrimidinyl group that is unsubstituted or substituted with one substituent selected from the group consisting of deuterium, a substituted or unsubstituted aryl group having 6 to 60 carbon atoms, and a substituted or unsubstituted heteroaryl group having 2 to 60 carbon atoms; or a benzofuropyrimidinyl group that is unsubstituted or substituted with one substituent selected from the group consisting of deuterium, a substituted or unsubstituted aryl group having 6 to 60 carbon atoms, and a substituted or unsubstituted heteroaryl group having 2 to 60 carbon atoms,
L1 is a substituted or unsubstituted arylene group having 6 to 60 carbon atoms,
Ar1 is an aryl group having 6 to 60 carbon atoms that is substituted with one or more cyano groups, and is optionally further substituted with deuterium,
Ar21 to Ar23 are the same as or different from each other, and are each independently hydrogen; deuterium; or a substituted or unsubstituted aryl group having 6 to 60 carbon atoms,
R1 and R2 are the same as or different from each other, and are each independently hydrogen; deuterium; or a substituted or unsubstituted alkyl group having 1 to 30 carbon atoms,
n is an integer from 1 to 4, and when n is 2 or higher, R1's are the same as or different from each other,
m is an integer from 1 to 3, and when m is 2 or higher, R2's are the same as or different from each other,
a1, a2, and a3 are each 0 or 1,
$$a 1 + a 2 + a 3 \geq 1 , and$$
$$a 1 + a 2 + a 3 + m = 4.$$

2. The compound of claim 1, wherein Chemical Formula 1 is any one of the following Chemical Formulae 1-1 to 1-5: 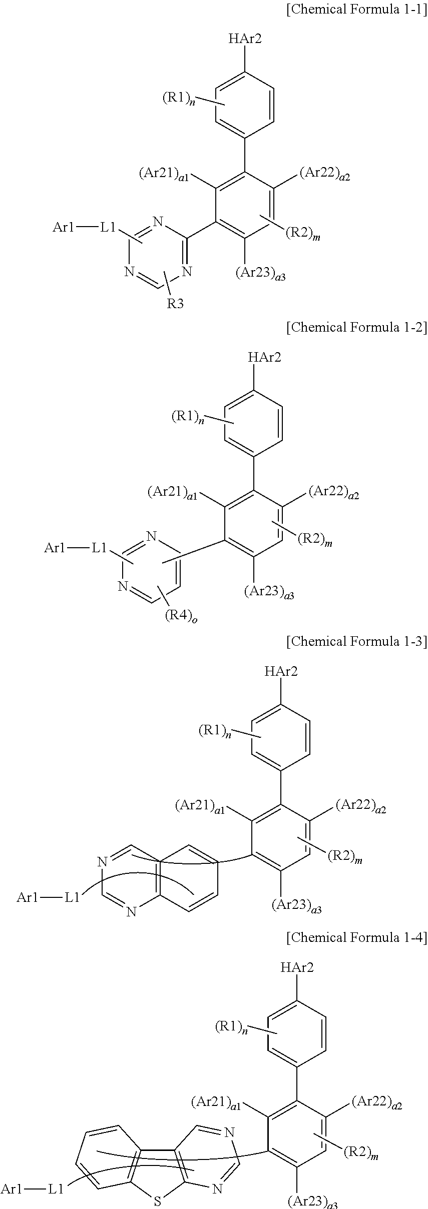 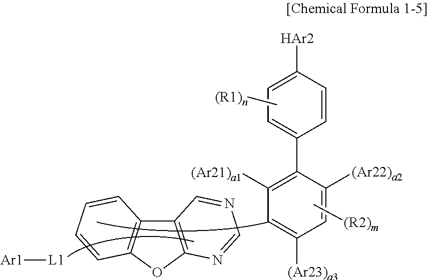
wherein in Chemical Formulae 1-1 to 1-5,
HAr2, L1, Ar1, Ar21 to Ar23, n, m, and a1 to a3 are the same as those defined in Chemical Formula 1,
R1 and R2 are the same as or different from each other, and are each independently hydrogen or deuterium,
R3 is deuterium; a substituted or unsubstituted aryl group having 6 to 60 carbon atoms; or a substituted or unsubstituted heteroaryl group having 2 to 60 carbon atoms,
R4 is hydrogen; deuterium; a substituted or unsubstituted alkyl group having 1 to 60 carbon atoms; a substituted or unsubstituted aryl group having 6 to 60 carbon atoms; or a substituted or unsubstituted heteroaryl group having 2 to 60 carbon atoms,
o is 1 or 2, and
when o is 2, R4's are the same as or different from each other.

3. The compound of claim 1, wherein Ar1 is a phenyl group that is substituted with one or more cyano groups, and is optionally further substituted with deuterium; a naphthyl group that is substituted with one or more cyano groups, and is optionally further substituted with deuterium; a biphenyl group that is substituted with one or more cyano groups, and is optionally further substituted with deuterium; or a terphenyl group that is substituted with one or more cyano groups, and is optionally further substituted with deuterium.

4. The compound of claim 1, wherein HAr1 is a divalent triazinyl group substituted with one substituent selected from the group consisting of deuterium, a substituted or unsubstituted phenyl group, a substituted or unsubstituted biphenyl group, a substituted or unsubstituted naphthyl group, and a substituted or unsubstituted pyrimidinyl group; a divalent pyrimidinyl group that is unsubstituted or substituted with one or two substituents each independently selected from the group consisting of deuterium, a substituted or unsubstituted phenyl group, a substituted or unsubstituted biphenyl group, a substituted or unsubstituted naphthyl group, a substituted or unsubstituted methyl group, a substituted or unsubstituted ethyl group, a substituted or unsubstituted propyl group, and a substituted or unsubstituted pyridinyl group; a divalent quinazolinyl group that is unsubstituted or substituted with deuterium; a divalent benzothienopyrimidinyl group that is unsubstituted or substituted with one substituent selected from the group consisting of deuterium, a substituted or unsubstituted phenyl group, a substituted or unsubstituted biphenyl group, a substituted or unsubstituted naphthyl group, and a substituted or unsubstituted pyrimidinyl group; or a divalent benzofuropyrimidinyl group that is unsubstituted or substituted with one substituent selected from the group consisting of deuterium, a substituted or unsubstituted phenyl group, a substituted or unsubstituted biphenyl group, a substituted or unsubstituted naphthyl group, and a substituted or unsubstituted pyrimidinyl group.

5. The compound of claim 1, wherein Ar21 to Ar23 are the same as or different from each other, and are each independently a substituted or unsubstituted phenyl group; a substituted or unsubstituted biphenyl group; a substituted or unsubstituted terphenyl group; a substituted or unsubstituted naphthyl group; a substituted or unsubstituted phenanthrenyl group; or a substituted or unsubstituted anthracenyl group.

6. The compound of claim 1, wherein L1 is a substituted or unsubstituted phenylene group; a substituted or unsubstituted divalent biphenyl group; a substituted or unsubstituted divalent terphenyl group; a substituted or unsubstituted divalent naphthyl group; a substituted or unsubstituted divalent anthracenyl group; or a substituted or unsubstituted divalent phenanthrenyl group.

7. The compound of claim 2, wherein R3 is a substituted or unsubstituted phenyl group; a substituted or unsubstituted biphenyl group; a substituted or unsubstituted naphthyl group; or a substituted or unsubstituted pyrimidinyl group.

8. The compound of claim 2, wherein R4 is hydrogen; deuterium; a substituted or unsubstituted phenyl group; a substituted or unsubstituted biphenyl group; a substituted or unsubstituted naphthyl group; a substituted or unsubstituted methyl group; a substituted or unsubstituted ethyl group; a substituted or unsubstituted propyl group; or a substituted or unsubstituted pyrimidinyl group.

9. The compound of claim 1, wherein HAr2 is a triazinyl group that is unsubstituted or substituted with two substituents each independently selected from the group consisting of deuterium, a substituted or unsubstituted phenyl group, a substituted or unsubstituted biphenyl group, a substituted or unsubstituted naphthyl group, and a substituted or unsubstituted pyrimidinyl group; a pyrimidinyl group that is unsubstituted or substituted with one to three substituents each independently selected from the group consisting of deuterium, a substituted or unsubstituted phenyl group, a substituted or unsubstituted biphenyl group, a substituted or unsubstituted naphthyl group, a substituted or unsubstituted methyl group, a substituted or unsubstituted ethyl group, a substituted or unsubstituted propyl group, and a substituted or unsubstituted pyridinyl group; a quinazolinyl group that is unsubstituted or substituted with one substituent selected from the group consisting of deuterium, a substituted or unsubstituted phenyl group, a substituted or unsubstituted biphenyl group, and a substituted or unsubstituted naphthyl group; a benzothienopyrimidinyl group that is unsubstituted or substituted with one substituent selected from the group consisting of deuterium, a substituted or unsubstituted phenyl group, a substituted or unsubstituted biphenyl group, a substituted or unsubstituted naphthyl group, a substituted or unsubstituted methyl group, a substituted or unsubstituted ethyl group, a substituted or unsubstituted propyl group, and a substituted or unsubstituted pyridinyl group; or a benzofuropyrimidinyl group that is unsubstituted or substituted with one substituent selected from the group consisting of deuterium, a substituted or unsubstituted phenyl group, a substituted or unsubstituted biphenyl group, a substituted or unsubstituted naphthyl group, a substituted or unsubstituted methyl group, a substituted or unsubstituted ethyl group, a substituted or unsubstituted propyl group, and a substituted or unsubstituted pyridinyl group.

10. The compound of claim 1, wherein Chemical Formula 1 is any one of the following structural formulae:

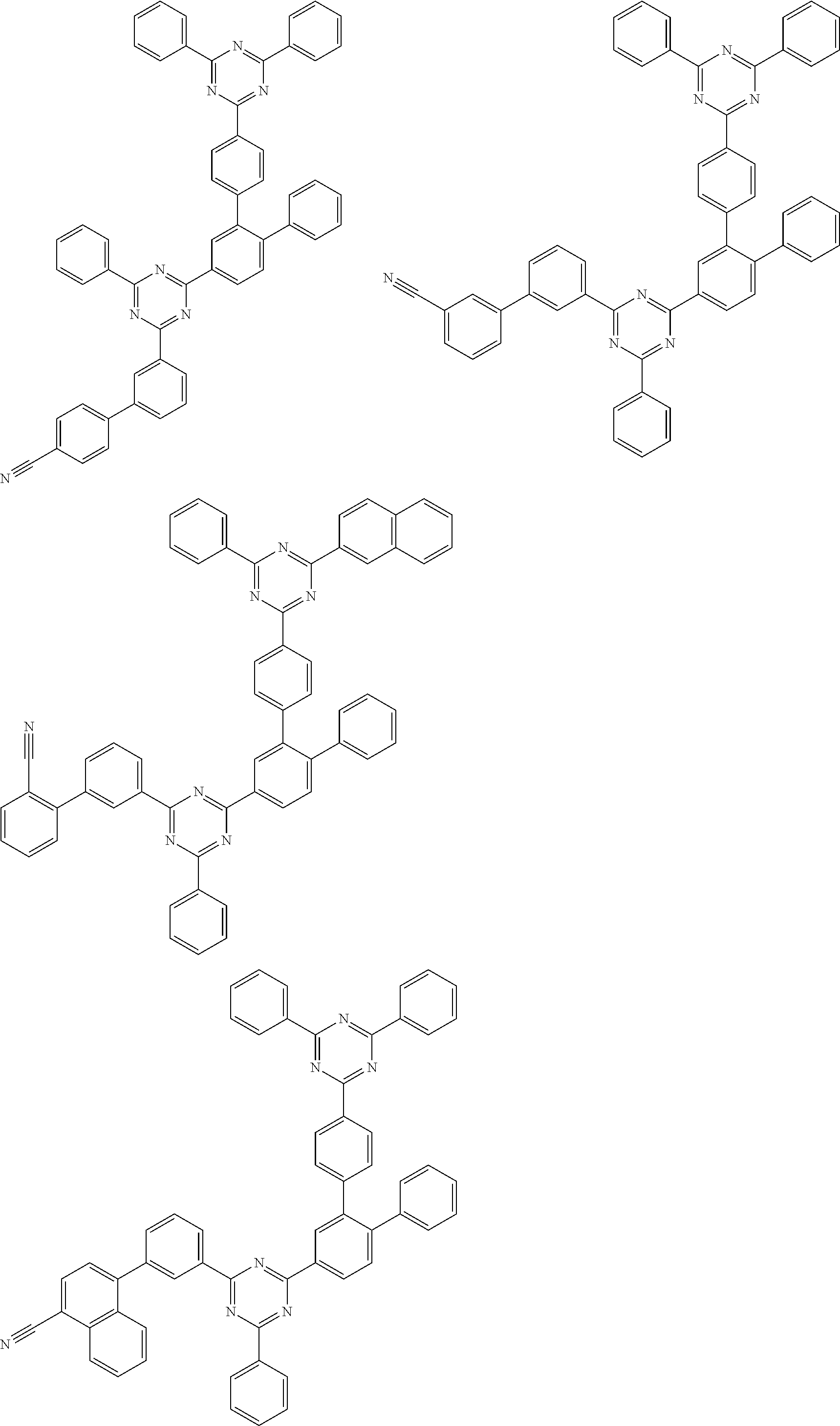

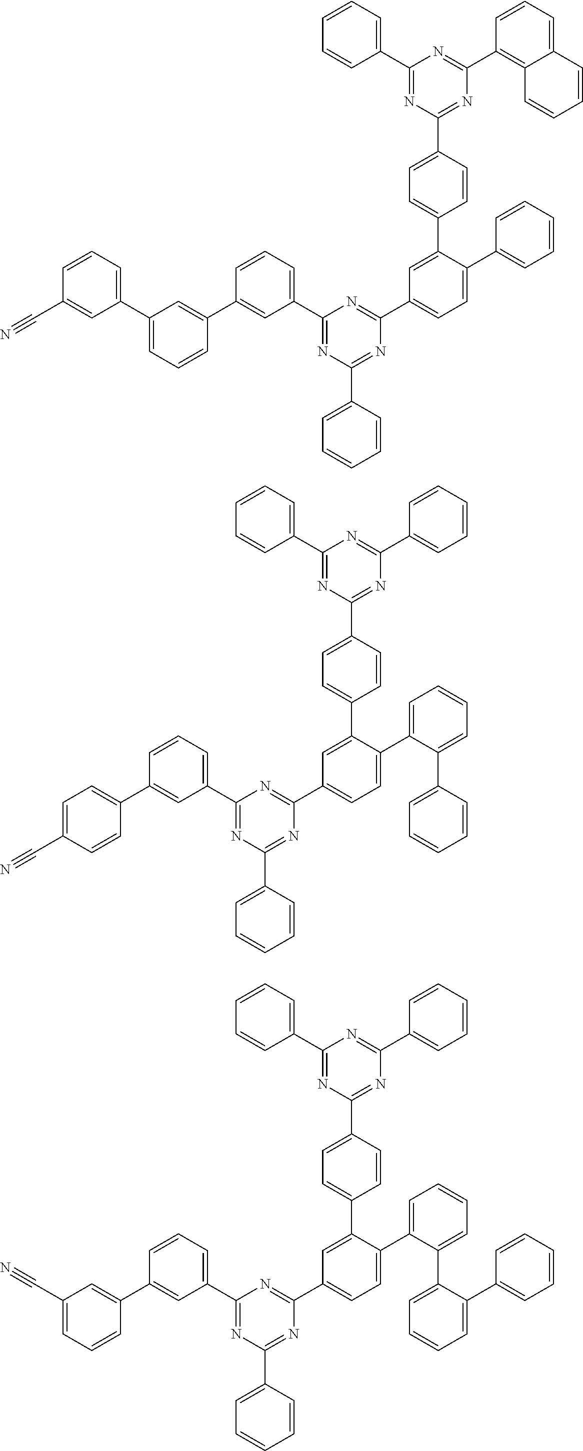

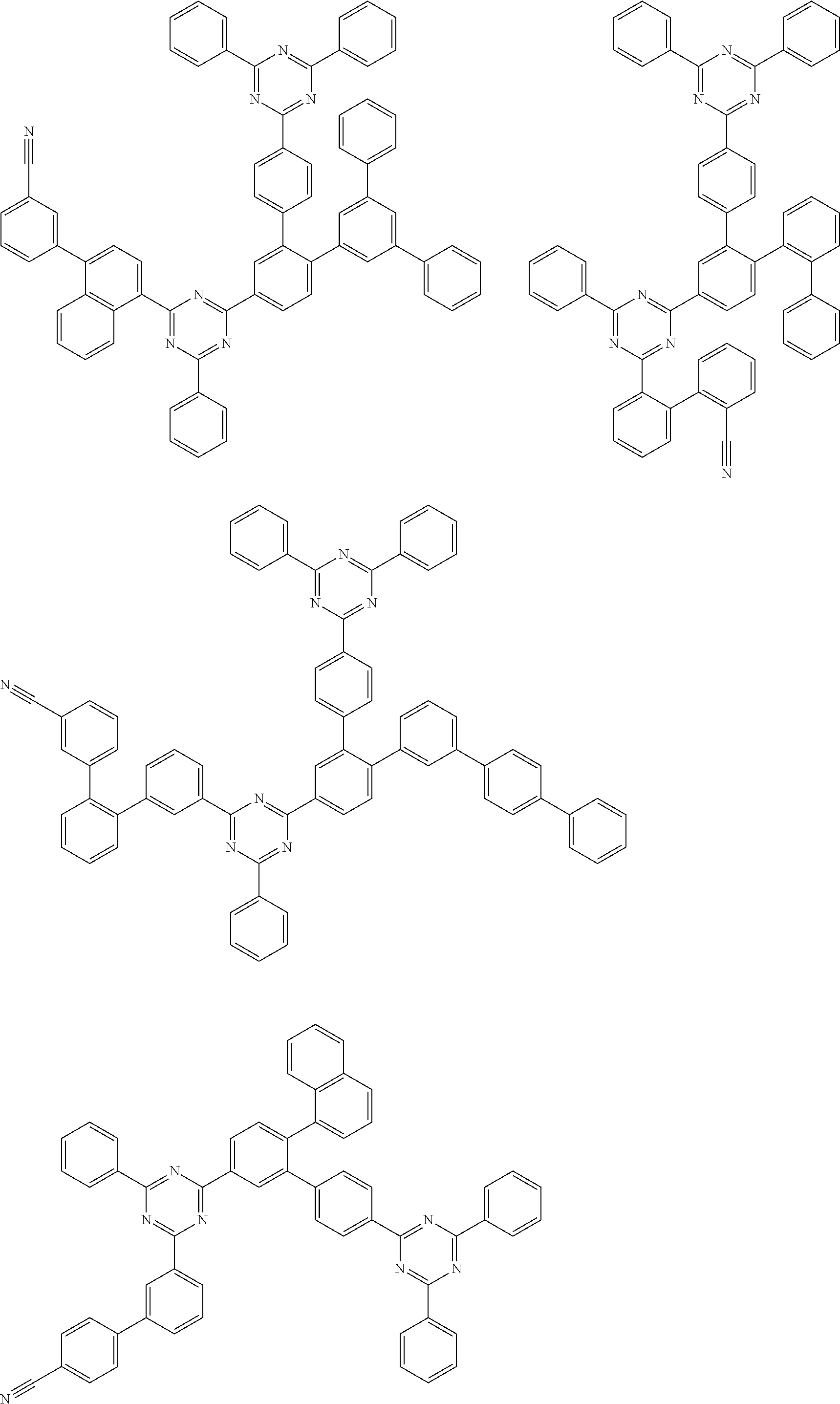

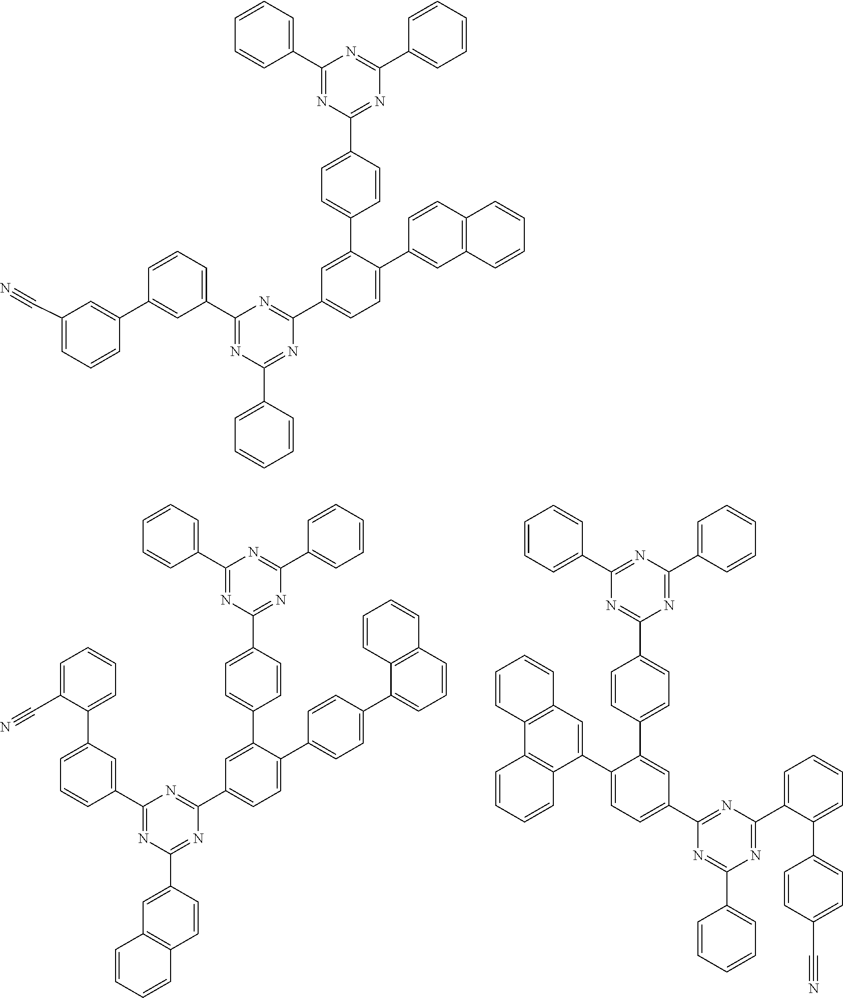 [SMILES: N#Cc1cccc(-c2cccc(-c3nc(-c4ccccc4)nc(-c4ccc(-c5ccc6ccccc6c5)c(-c5ccc(-c6nc(-c7ccccc7)nc(-c7ccccc7)n6)cc5)c4)n3)c2)c1 ; N#Cc1ccccc1-c1cccc(-c2nc(-c3ccc(-c4ccc(-c5cccc6ccccc56)cc4)c(-c4ccc(-c5nc(-c6ccccc6)nc(-c6ccccc6)n5)cc4)c3)nc(-c3ccc4ccccc4c3)n2)c1 ; N#Cc1ccc(-c2ccccc2-c2nc(-c3ccccc3)nc(-c3ccc(-c4cc5ccccc5c5ccccc45)c(-c4ccc(-c5nc(-c6ccccc6)nc(-c6ccccc6)n5)cc4)c3)n2)cc1]

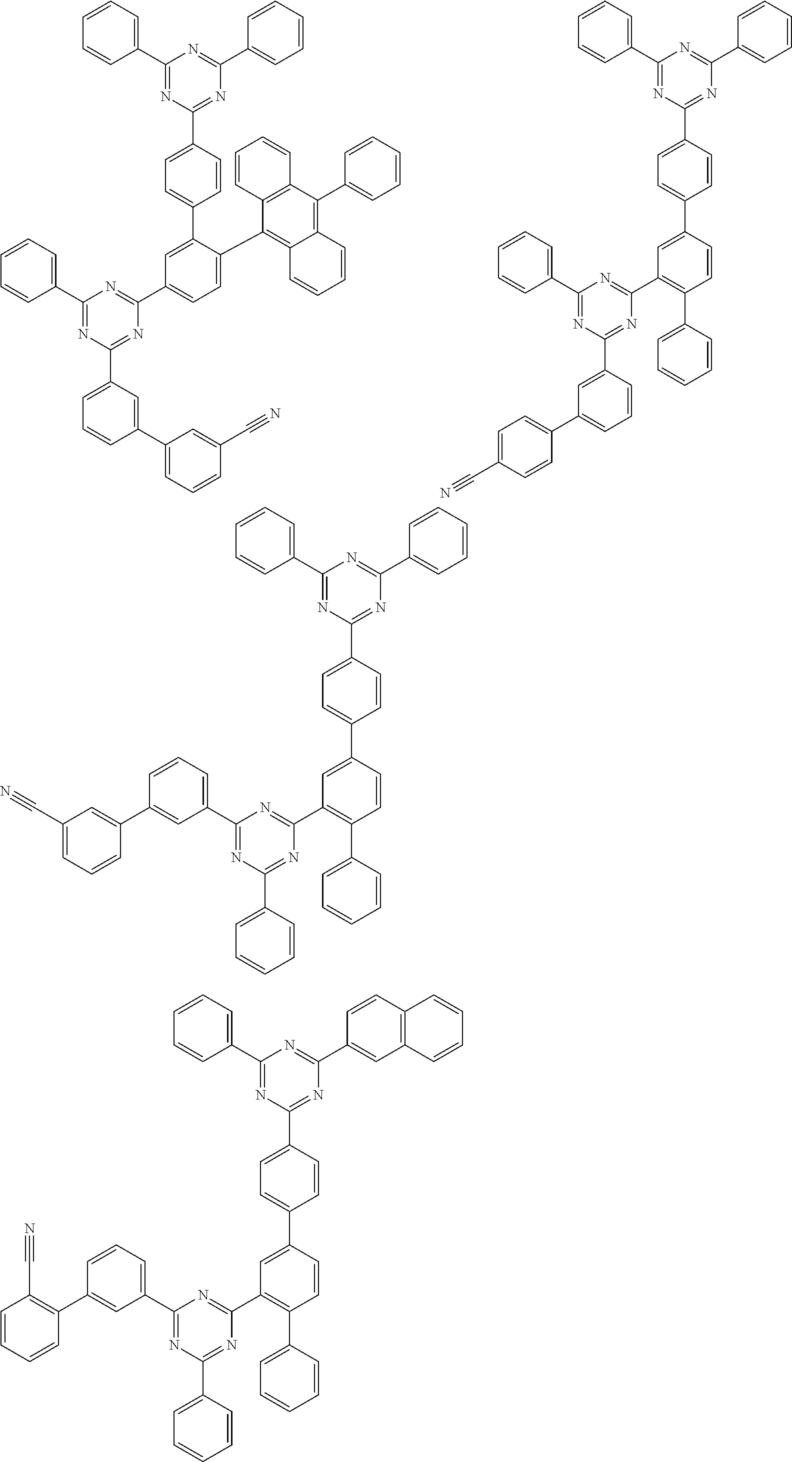

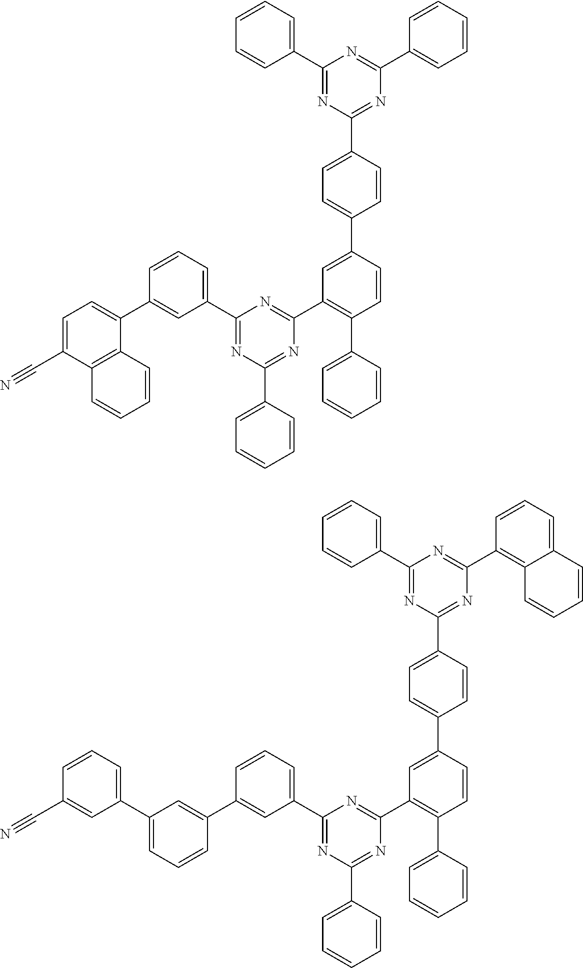 [SMILES: N#Cc1ccc(-c2cccc(-c3nc(-c4ccccc4)nc(-c4cc(-c5ccc(-c6nc(-c7ccccc7)nc(-c7ccccc7)n6)cc5)ccc4-c4ccccc4)n3)c2)c2ccccc12 ; N#Cc1cccc(-c2cccc(-c3cccc(-c4nc(-c5ccccc5)nc(-c5cc(-c6ccc(-c7nc(-c8ccccc8)nc(-c8cccc9ccccc89)n7)cc6)ccc5-c5ccccc5)n4)c3)c2)c1]

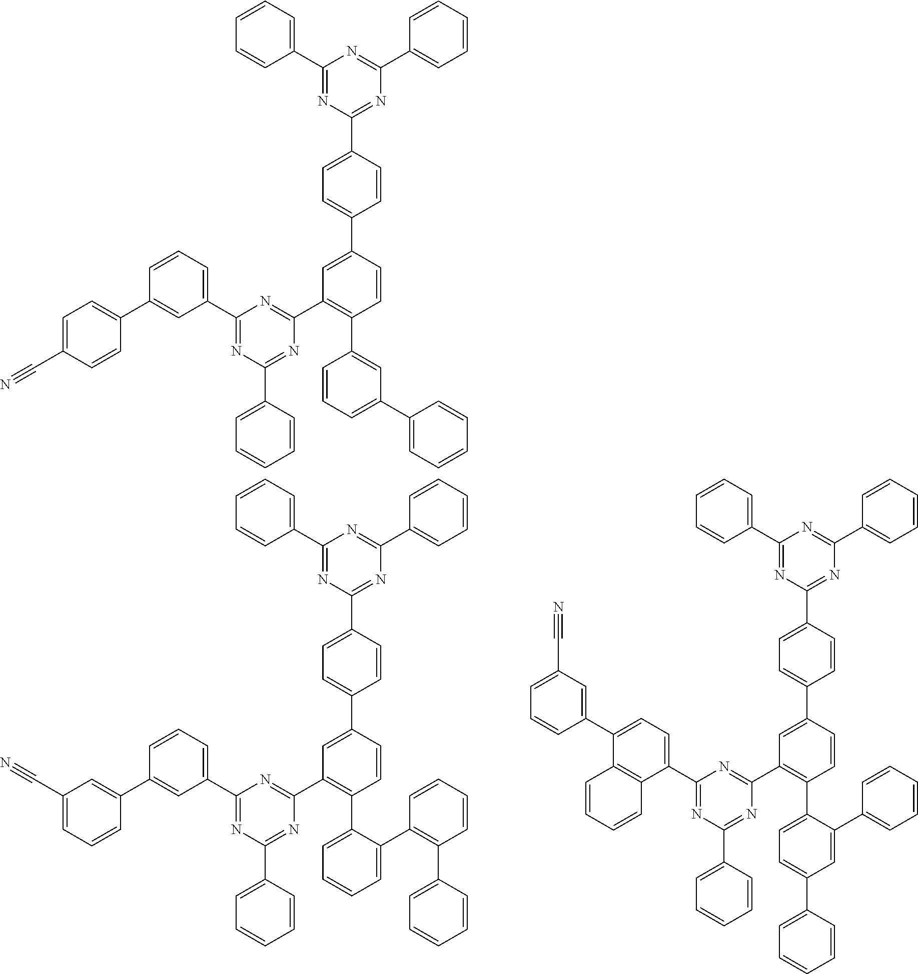

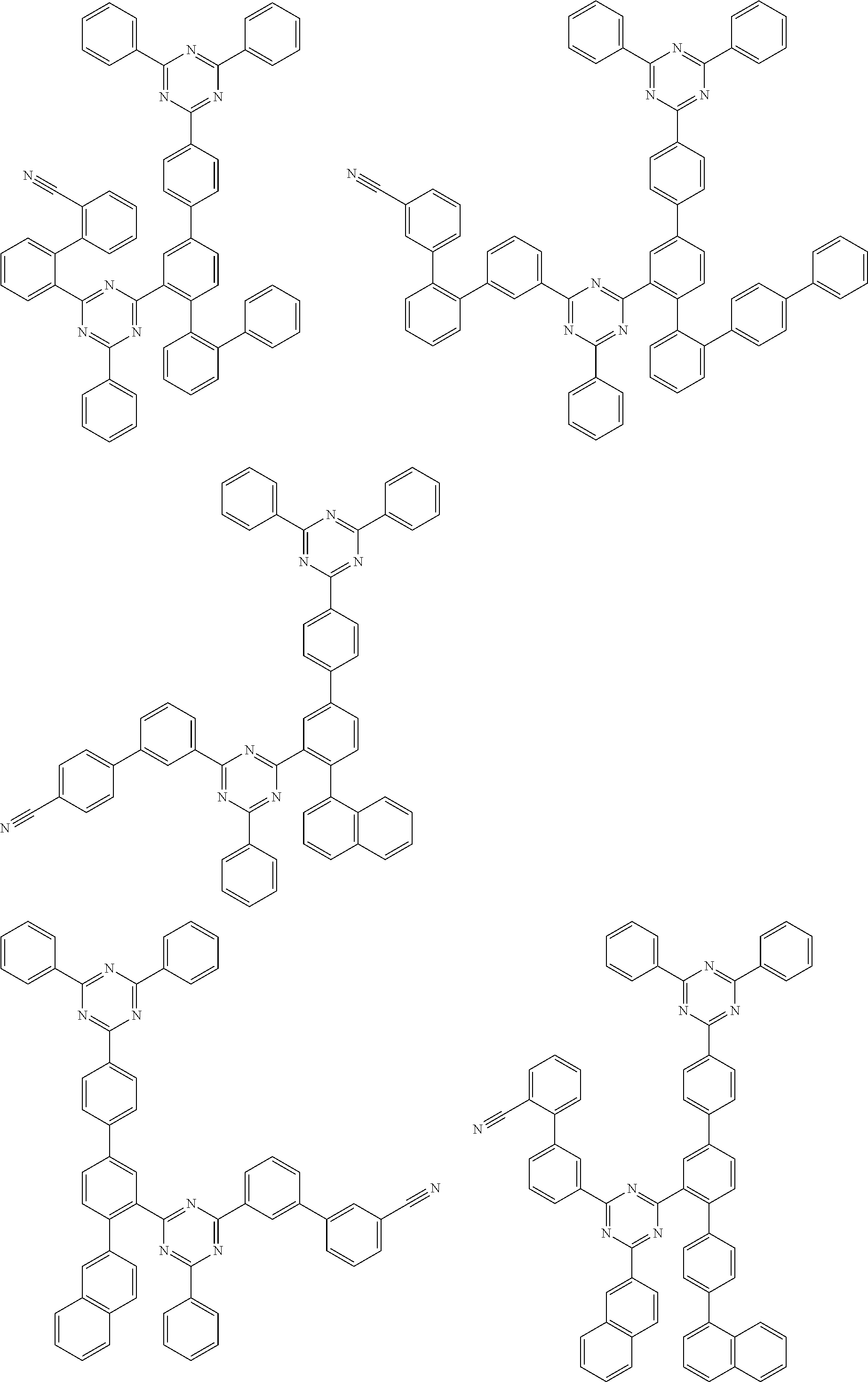

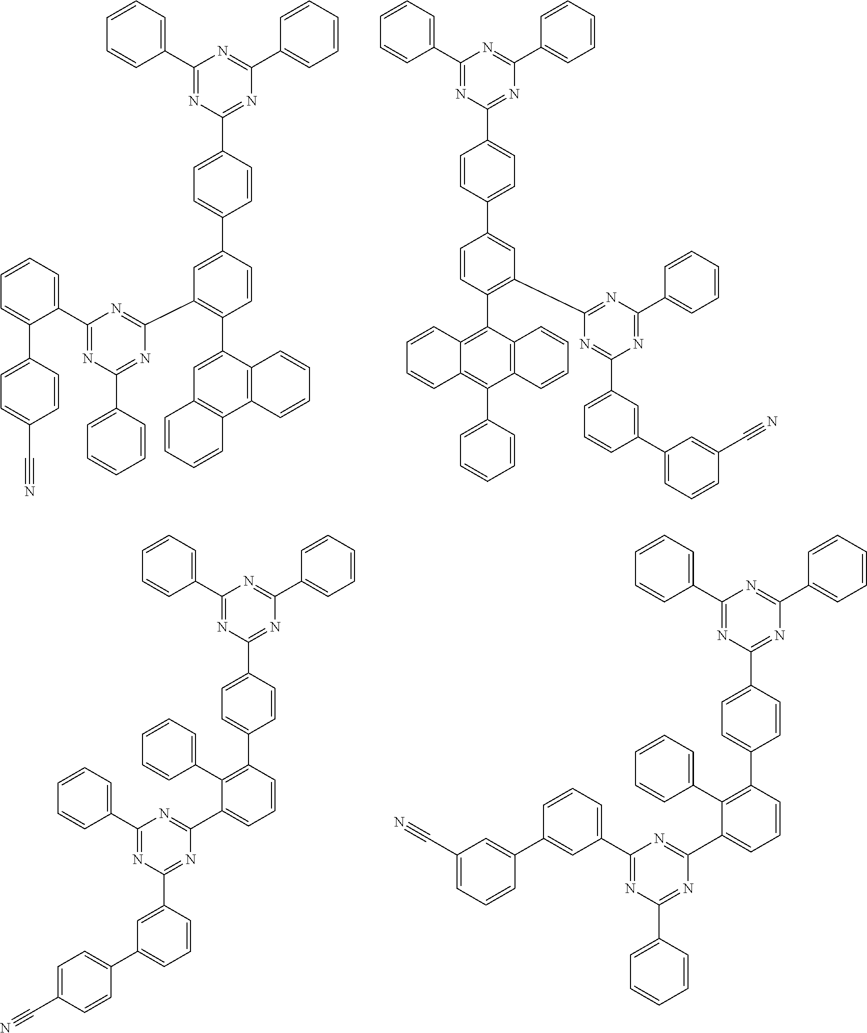

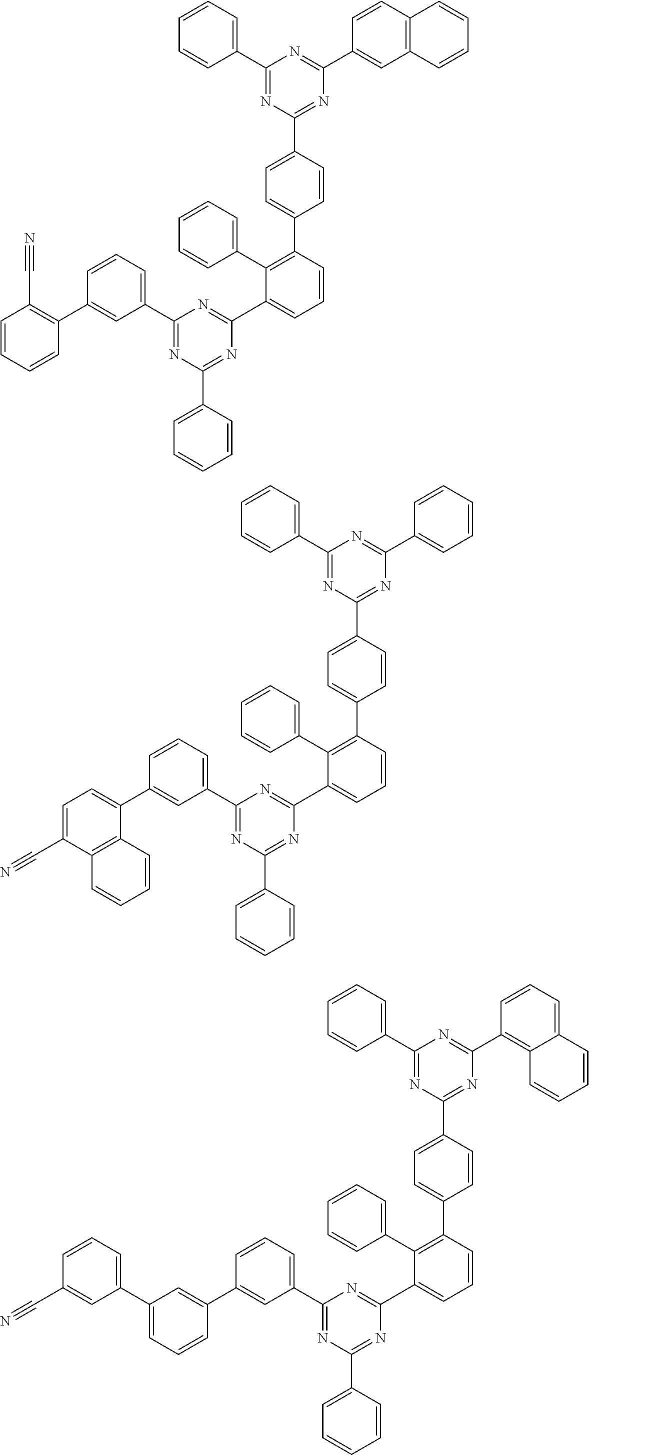 [SMILES: N#Cc1ccccc1-c1cccc(-c2nc(-c3ccccc3)nc(-c3cccc(-c4ccc(-c5nc(-c6ccccc6)nc(-c6ccc7ccccc7c6)n5)cc4)c3-c3ccccc3)n2)c1 ; N#Cc1ccc(-c2cccc(-c3nc(-c4ccccc4)nc(-c4cccc(-c5ccc(-c6nc(-c7ccccc7)nc(-c7ccccc7)n6)cc5)c4-c4ccccc4)n3)c2)c2ccccc12 ; N#Cc1cccc(-c2cccc(-c3cccc(-c4nc(-c5ccccc5)nc(-c5cccc(-c6ccc(-c7nc(-c8ccccc8)nc(-c8cccc9ccccc89)n7)cc6)c5-c5ccccc5)n4)c3)c2)c1]

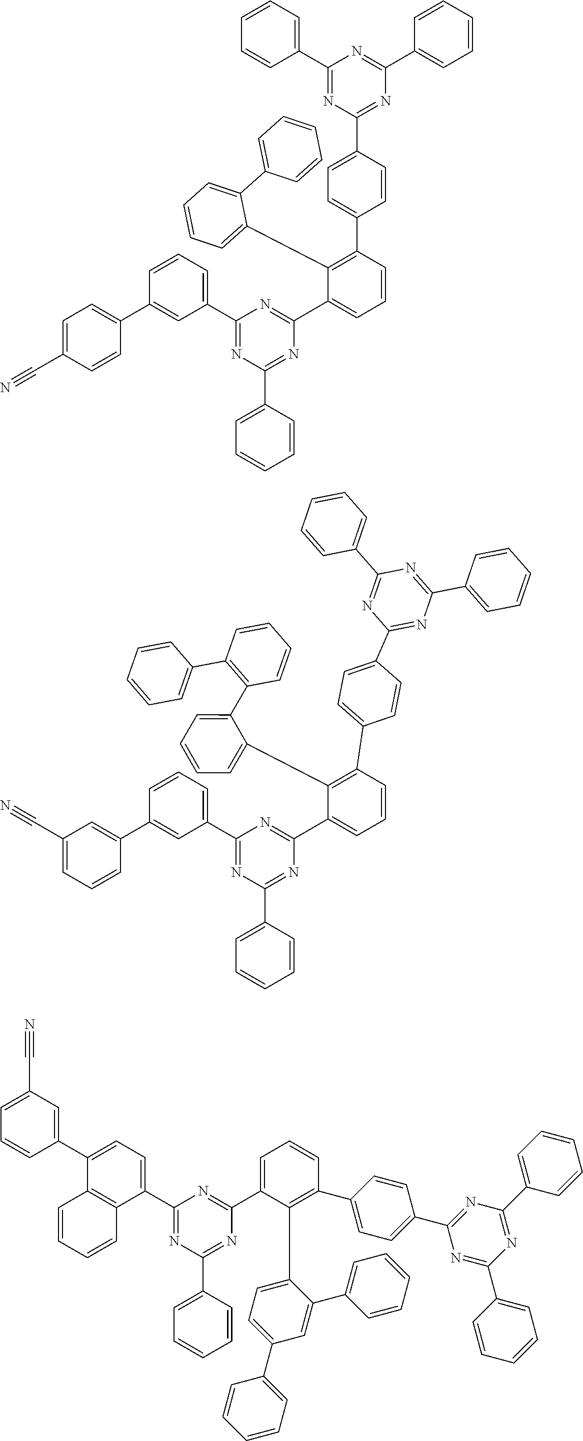 [SMILES: N#Cc1ccc(-c2cccc(-c3nc(-c4ccccc4)nc(-c4cccc(-c5ccc(-c6nc(-c7ccccc7)nc(-c7ccccc7)n6)cc5)c4-c4ccccc4-c4ccccc4)n3)c2)cc1 ; N#Cc1cccc(-c2cccc(-c3nc(-c4ccccc4)nc(-c4cccc(-c5ccc(-c6nc(-c7ccccc7)nc(-c7ccccc7)n6)cc5)c4-c4ccccc4-c4ccccc4-c4ccccc4)n3)c2)c1 ; N#Cc1cccc(-c2ccc(-c3nc(-c4ccccc4)nc(-c4cccc(-c5ccc(-c6nc(-c7ccccc7)nc(-c7ccccc7)n6)cc5)c4-c4ccc(-c5ccccc5)cc4-c4ccccc4)n3)c3ccccc23)c1]

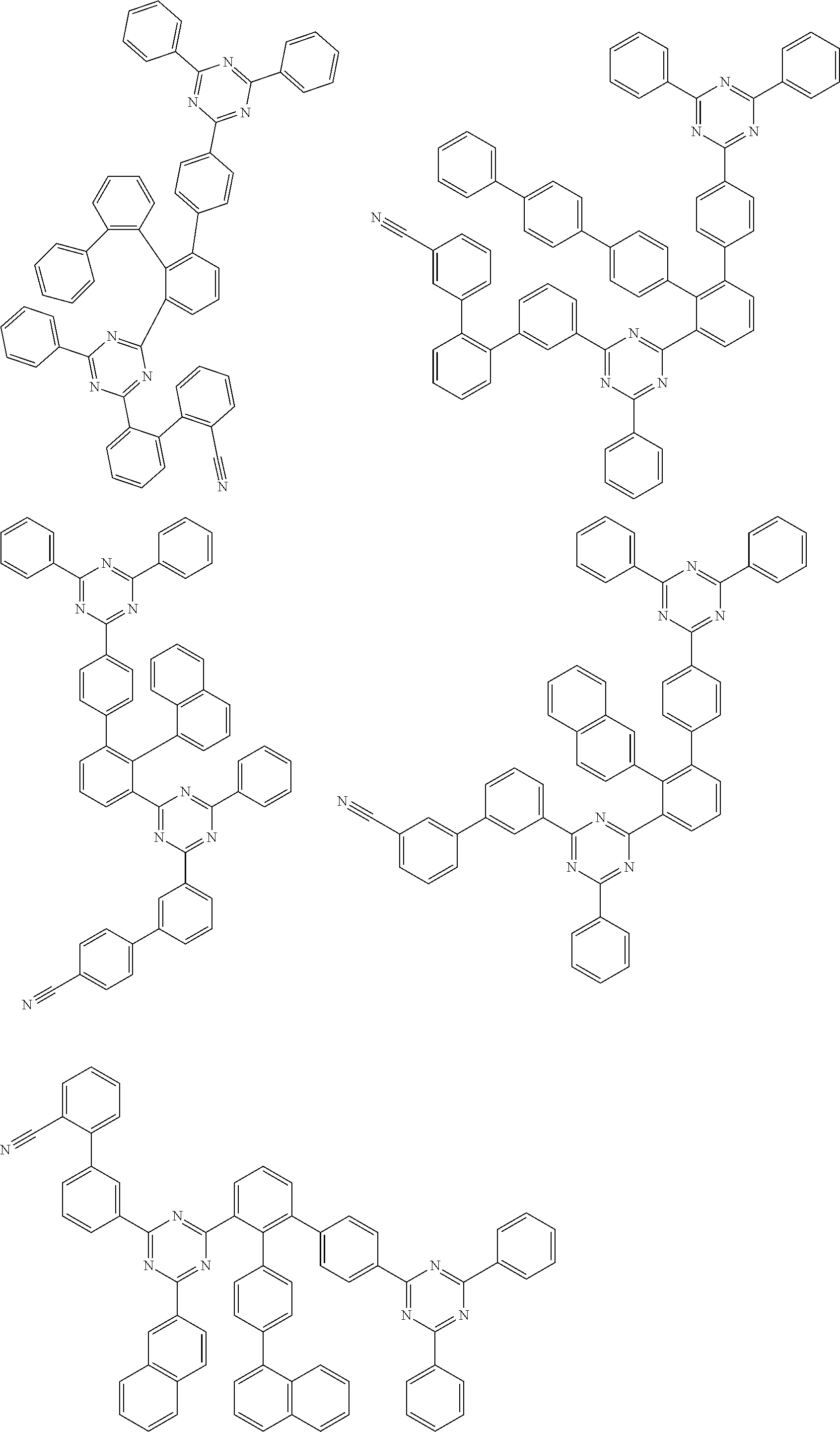

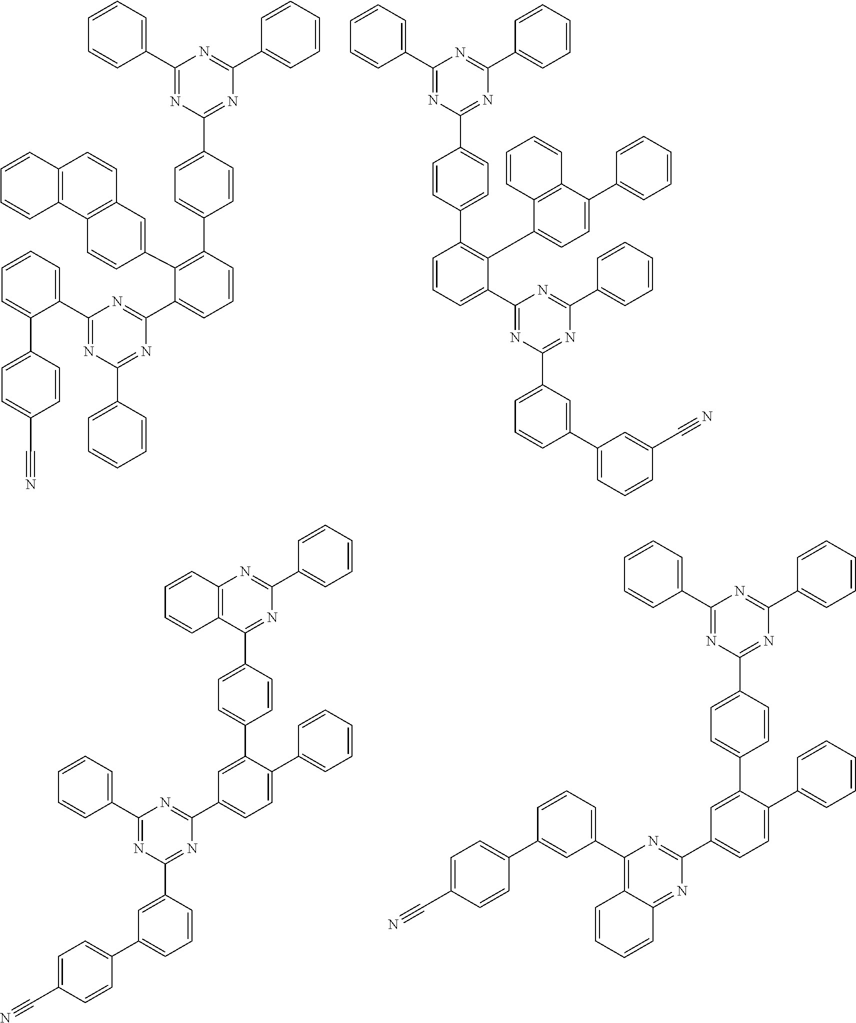

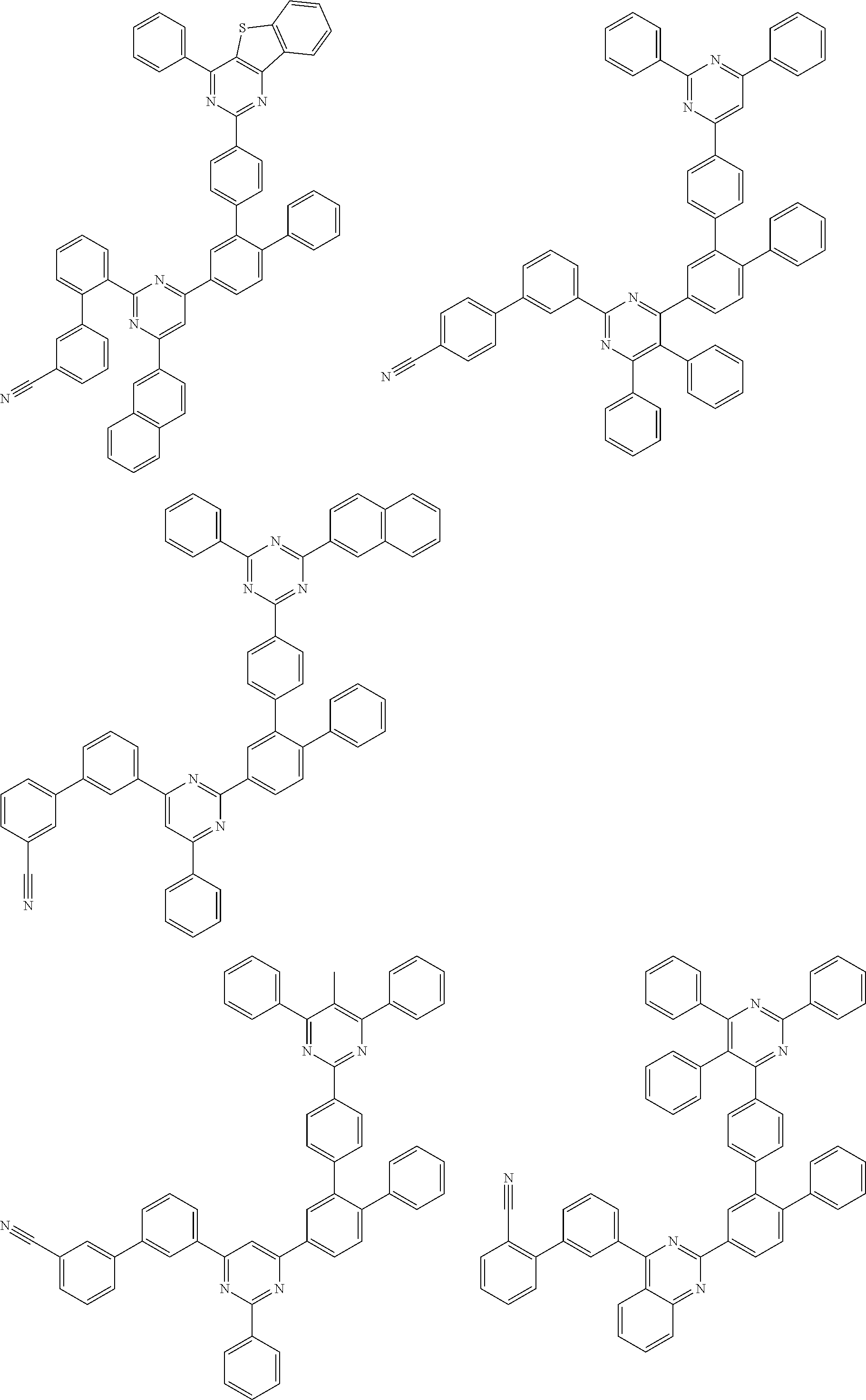

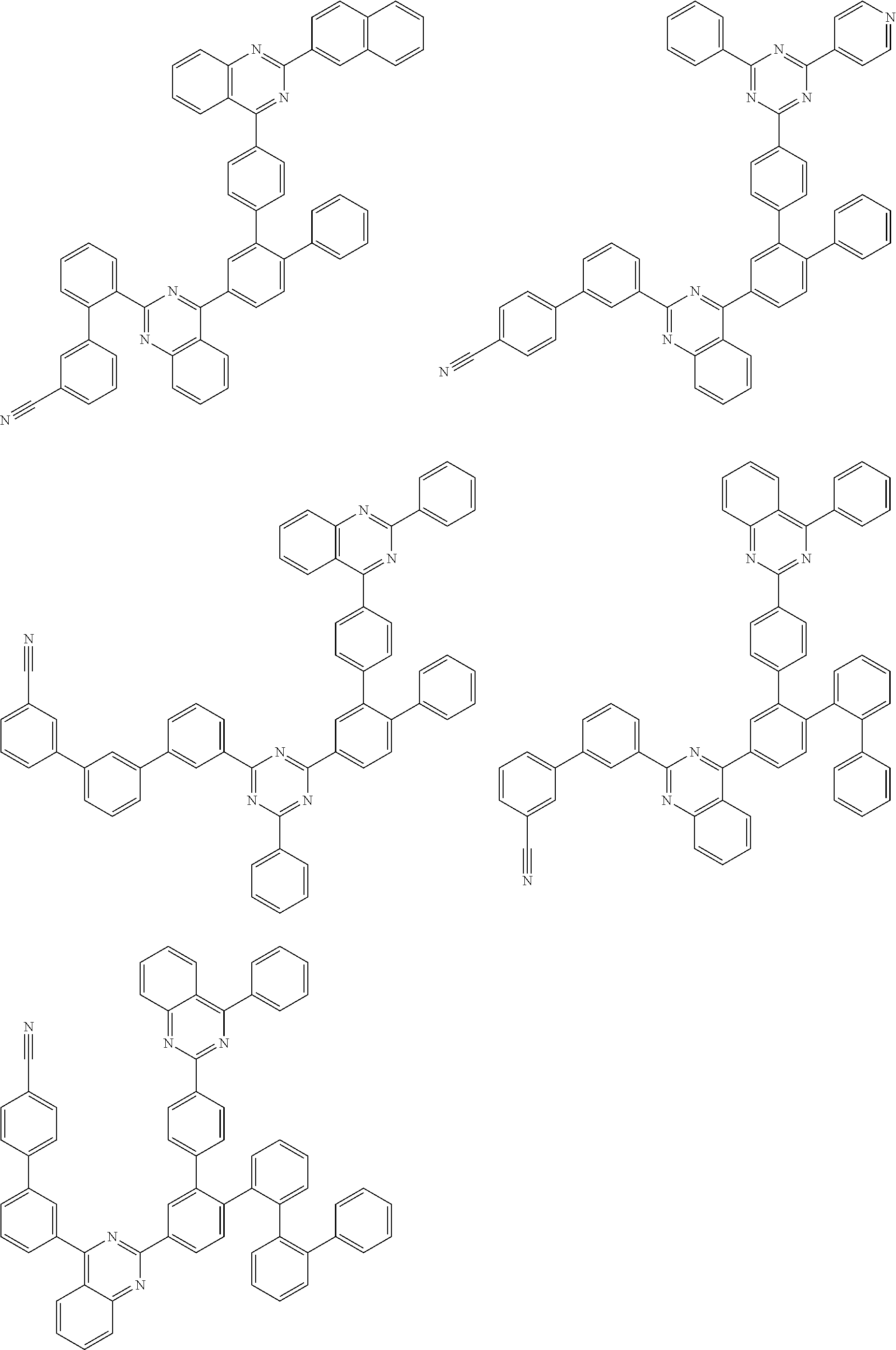

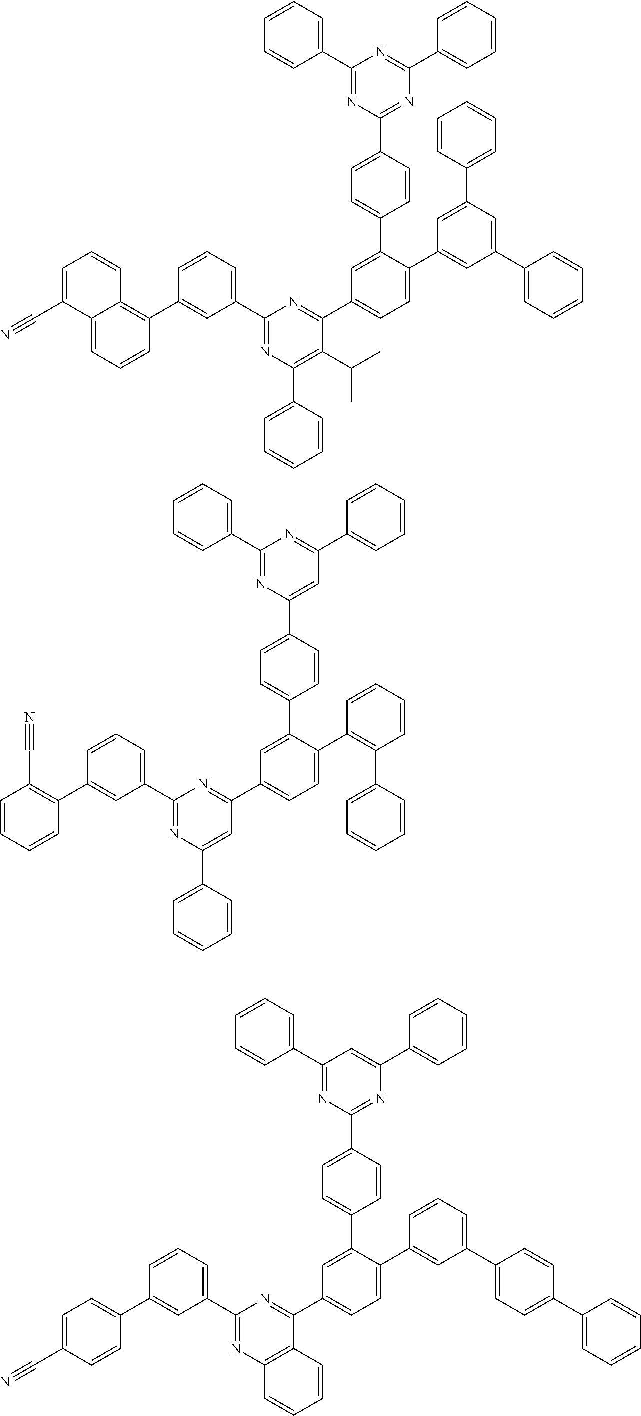 [SMILES: CC(C)c1c(-c2ccccc2)nc(-c2cccc(-c3cccc4c(C#N)cccc34)c2)nc1-c1ccc(-c2cc(-c3ccccc3)cc(-c3ccccc3)c2)c(-c2ccc(-c3nc(-c4ccccc4)nc(-c4ccccc4)n3)cc2)c1 ; N#Cc1ccccc1-c1cccc(-c2nc(-c3ccccc3)cc(-c3ccc(-c4ccccc4-c4ccccc4)c(-c4ccc(-c5cc(-c6ccccc6)nc(-c6ccccc6)n5)cc4)c3)n2)c1 ; N#Cc1ccc(-c2cccc(-c3nc(-c4ccc(-c5cccc(-c6ccc(-c7ccccc7)cc6)c5)c(-c5ccc(-c6nc(-c7ccccc7)cc(-c7ccccc7)n6)cc5)c4)c4ccccc4n3)c2)cc1]

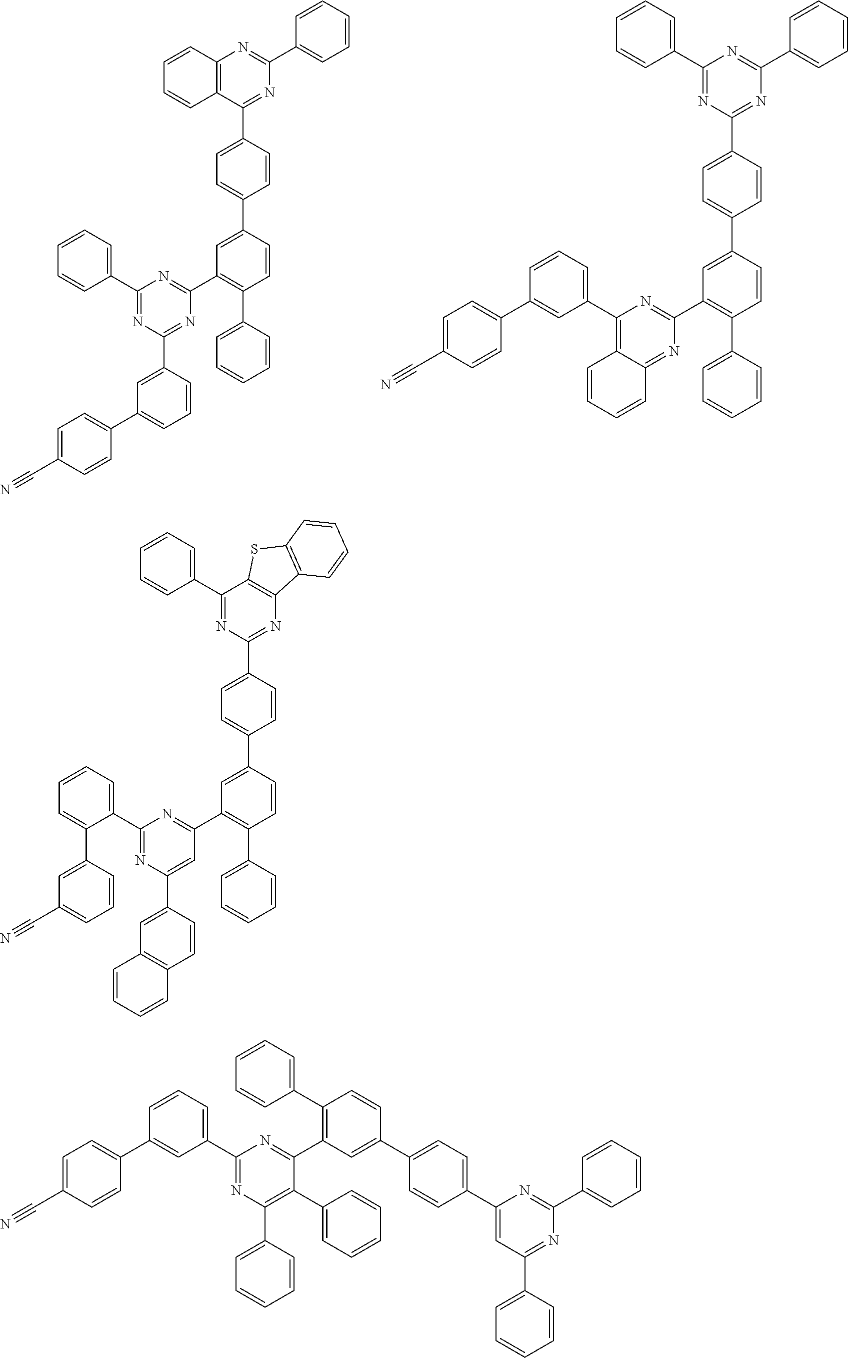 [SMILES: N#Cc1ccc(-c2cccc(-c3nc(-c4ccccc4)nc(-c4cc(-c5ccc(-c6nc(-c7ccccc7)nc7ccccc67)cc5)ccc4-c4ccccc4)n3)c2)cc1 ; N#Cc1ccc(-c2cccc(-c3nc(-c4cc(-c5ccc(-c6nc(-c7ccccc7)nc(-c7ccccc7)n6)cc5)ccc4-c4ccccc4)nc4ccccc34)c2)cc1 ; N#Cc1cccc(-c2ccccc2-c2nc(-c3ccc4ccccc4c3)cc(-c3cc(-c4ccc(-c5nc(-c6ccccc6)c6sc7ccccc7c6n5)cc4)ccc3-c3ccccc3)n2)c1 ; N#Cc1ccc(-c2cccc(-c3nc(-c4ccccc4)c(-c4ccccc4)c(-c4cc(-c5ccc(-c6cc(-c7ccccc7)nc(-c7ccccc7)n6)cc5)ccc4-c4ccccc4)n3)c2)cc1]

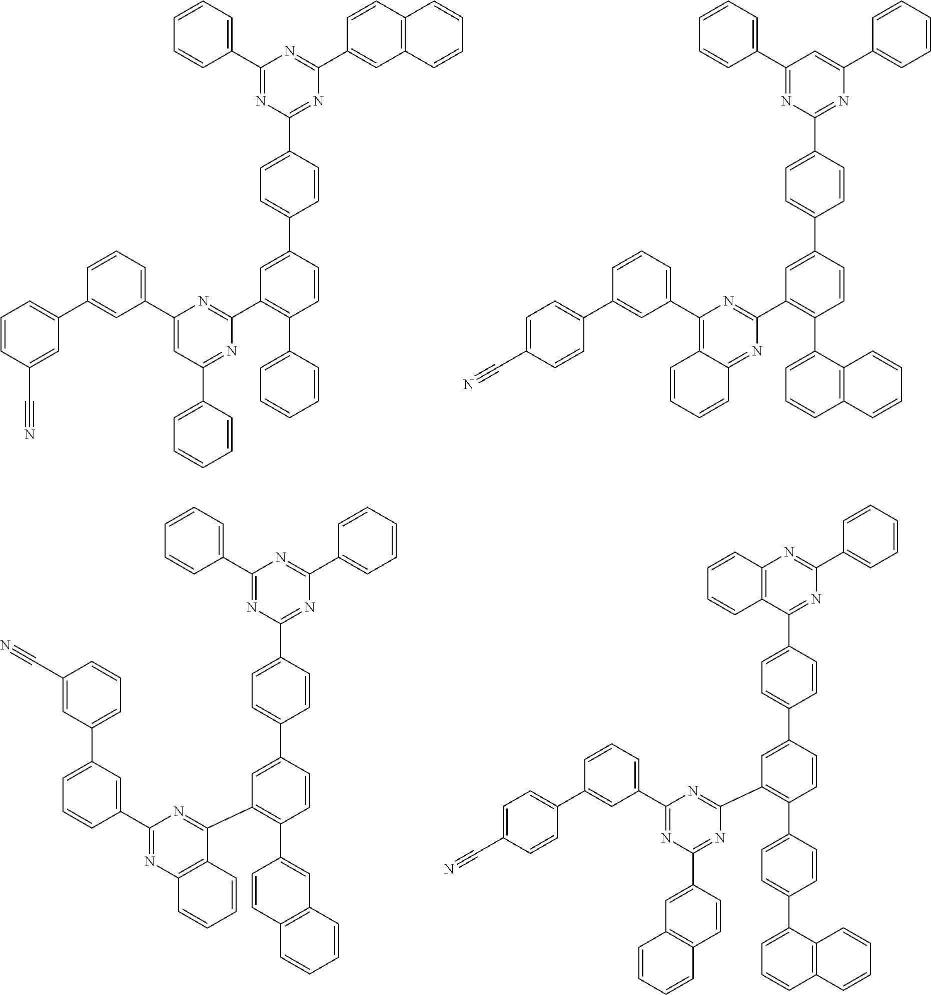

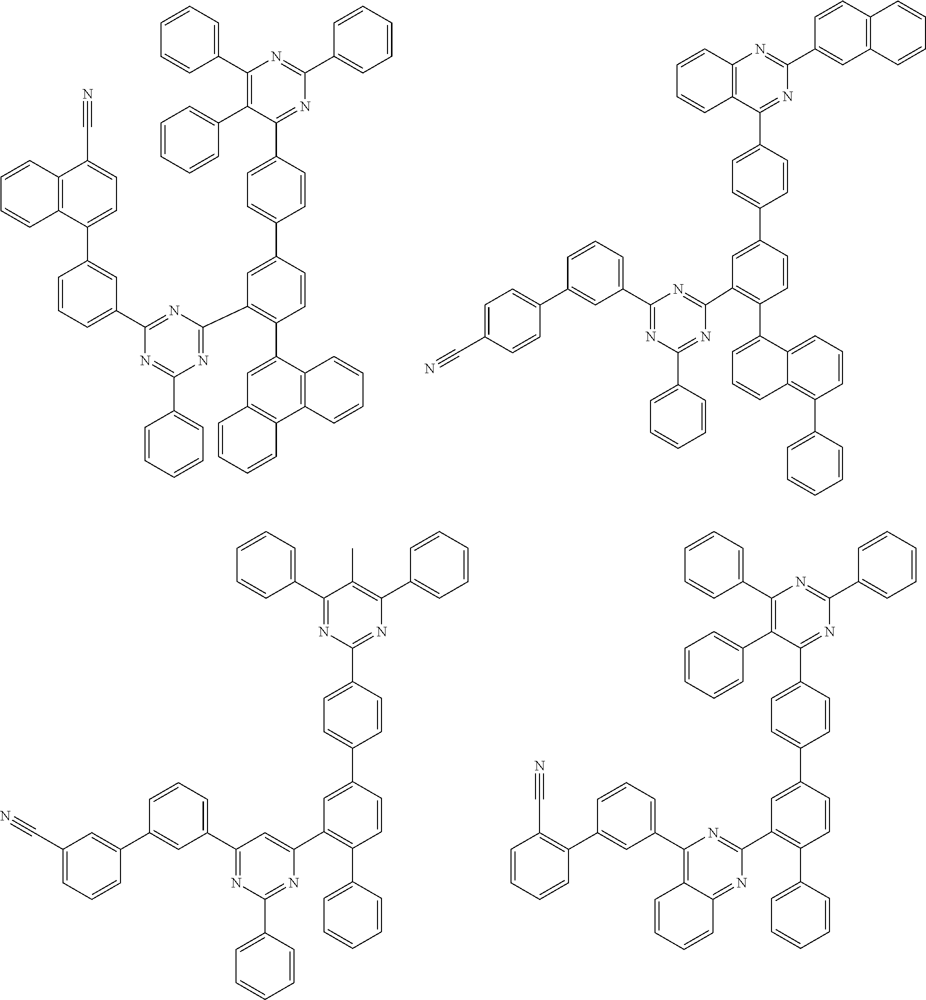 [SMILES: N#Cc1ccc(-c2cccc(-c3nc(-c4ccccc4)nc(-c4cc(-c5ccc(-c6nc(-c7ccccc7)nc(-c7ccccc7)c6-c6ccccc6)cc5)ccc4-c4cc5ccccc5c5ccccc45)n3)c2)c2ccccc12 ; N#Cc1ccc(-c2cccc(-c3nc(-c4ccccc4)nc(-c4cc(-c5ccc(-c6nc(-c7ccc8ccccc8c7)nc7ccccc67)cc5)ccc4-c4cccc5c(-c6ccccc6)cccc45)n3)c2)cc1 ; Cc1c(-c2ccccc2)nc(-c2ccc(-c3ccc(-c4ccccc4)c(-c4cc(-c5cccc(-c6cccc(C#N)c6)c5)nc(-c5ccccc5)n4)c3)cc2)nc1-c1ccccc1 ; N#Cc1ccccc1-c1cccc(-c2nc(-c3cc(-c4ccc(-c5nc(-c6ccccc6)nc(-c6ccccc6)c5-c5ccccc5)cc4)ccc3-c3ccccc3)nc3ccccc23)c1]

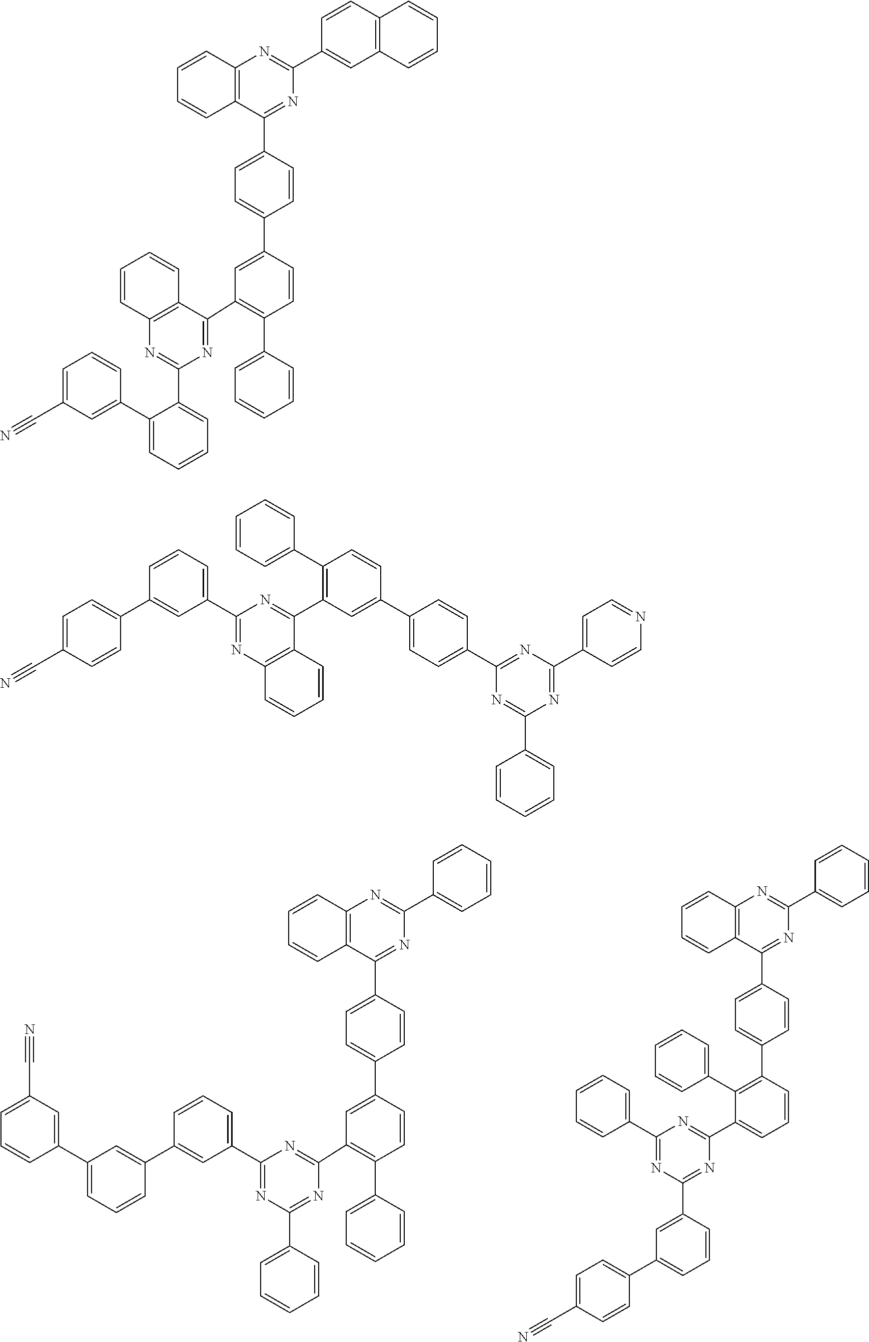 [SMILES: N#Cc1cccc(-c2ccccc2-c2nc(-c3cc(-c4ccc(-c5nc(-c6ccc7ccccc7c6)nc6ccccc56)cc4)ccc3-c3ccccc3)c3ccccc3n2)c1 ; N#Cc1ccc(-c2cccc(-c3nc(-c4cc(-c5ccc(-c6nc(-c7ccccc7)nc(-c7ccncc7)n6)cc5)ccc4-c4ccccc4)c4ccccc4n3)c2)cc1 ; N#Cc1cccc(-c2cccc(-c3cccc(-c4nc(-c5ccccc5)nc(-c5cc(-c6ccc(-c7nc(-c8ccccc8)nc8ccccc78)cc6)ccc5-c5ccccc5)n4)c3)c2)c1 ; N#Cc1ccc(-c2cccc(-c3nc(-c4ccccc4)nc(-c4cccc(-c5ccc(-c6nc(-c7ccccc7)nc7ccccc67)cc5)c4-c4ccccc4)n3)c2)cc1]

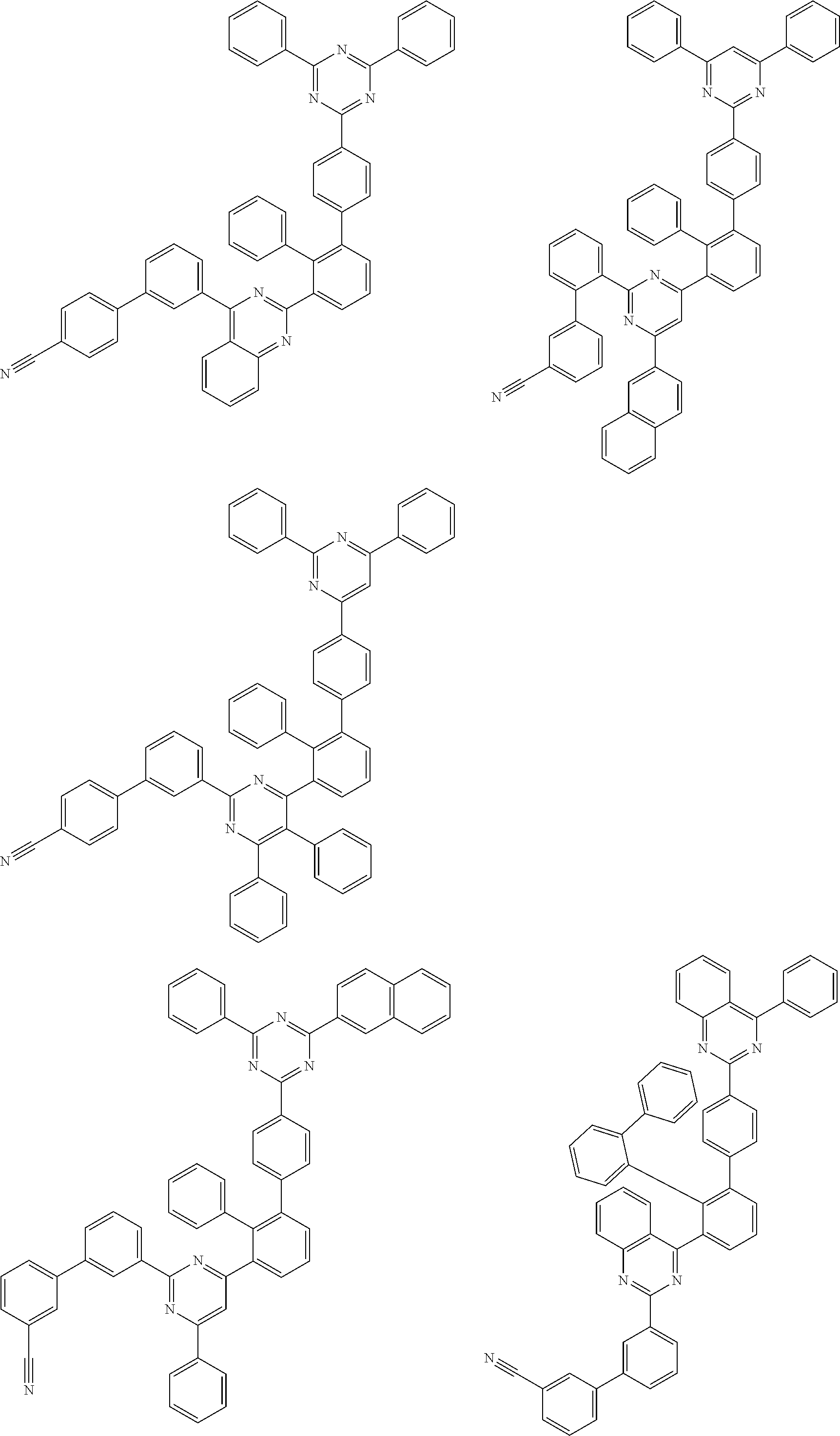

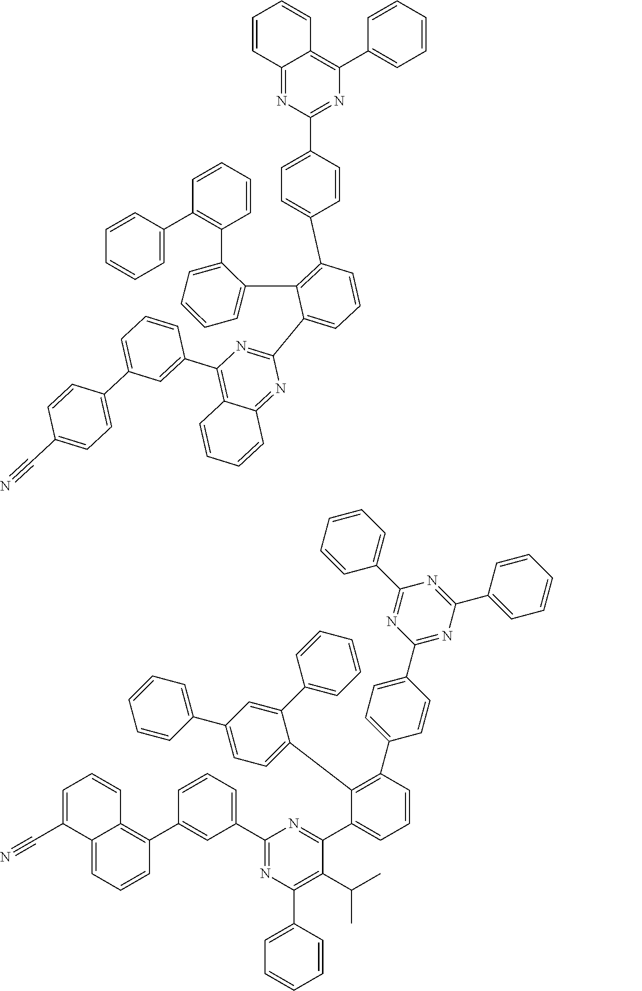 [SMILES: N#Cc1ccc(-c2cccc(-c3nc(-c4cccc(-c5ccc(-c6nc(-c7ccccc7)c7ccccc7n6)cc5)c4-c4ccccc4-c4ccccc4-c4ccccc4)nc4ccccc34)c2)cc1 ; CC(C)c1c(-c2ccccc2)nc(-c2cccc(-c3cccc4c(C#N)cccc34)c2)nc1-c1cccc(-c2ccc(-c3nc(-c4ccccc4)nc(-c4ccccc4)n3)cc2)c1-c1ccc(-c2ccccc2)cc1-c1ccccc1]

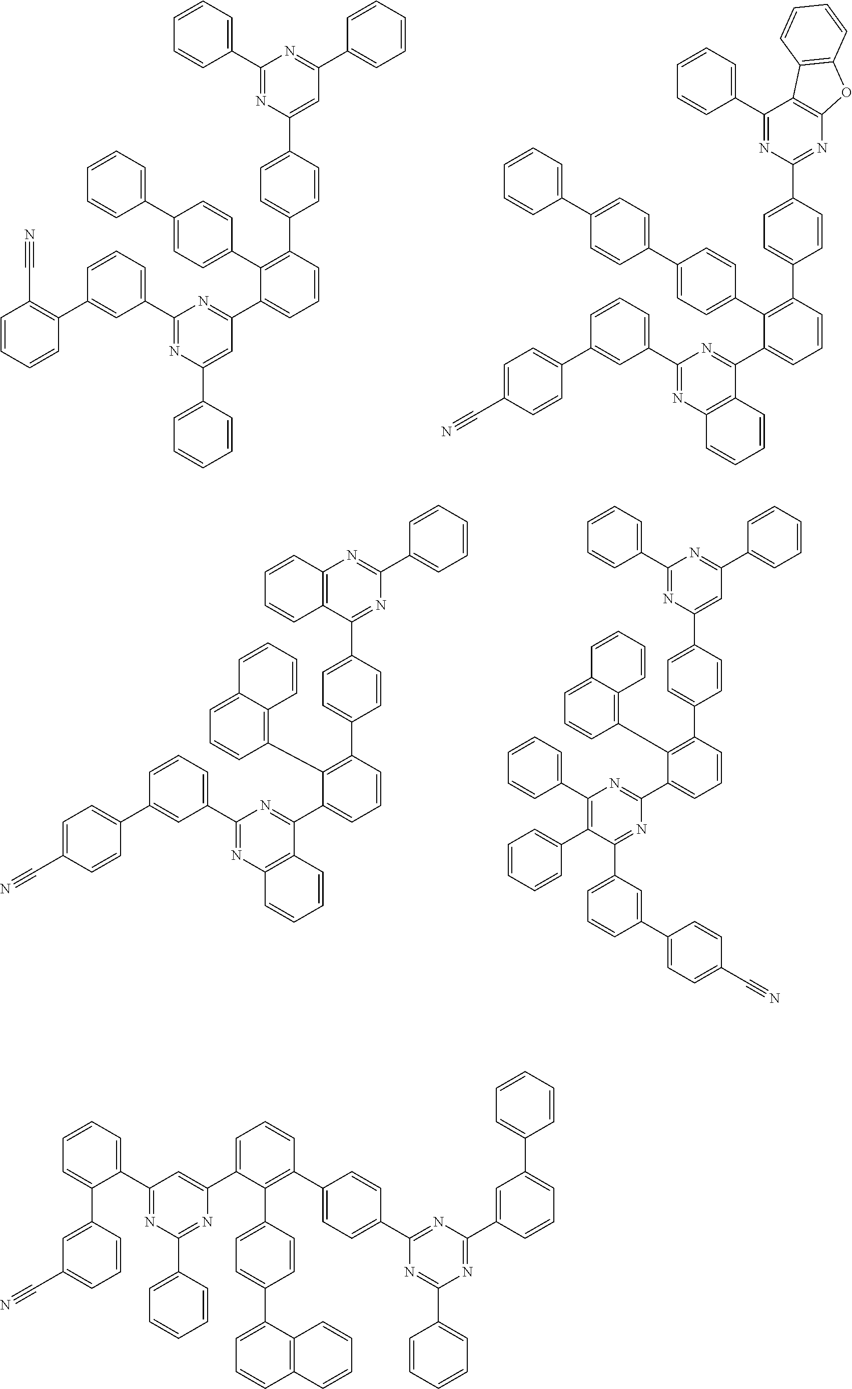

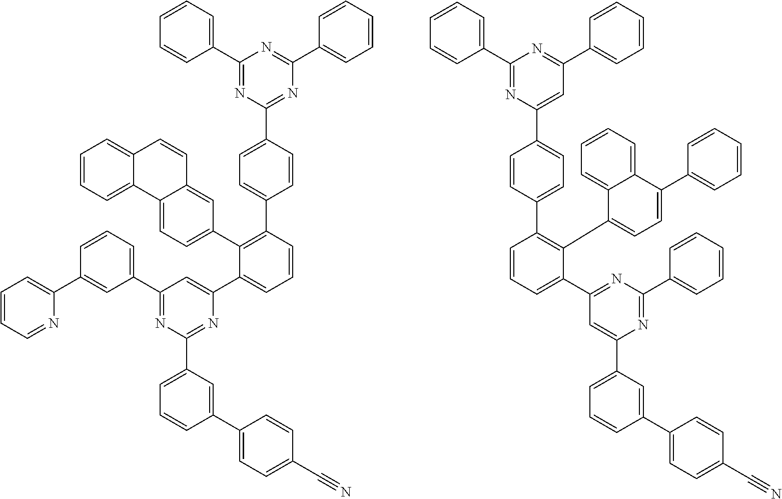 [SMILES: N#Cc1ccc(-c2cccc(-c3nc(-c4cccc(-c5ccccn5)c4)cc(-c4cccc(-c5ccc(-c6nc(-c7ccccc7)nc(-c7ccccc7)n6)cc5)c4-c4ccc5c(ccc6ccccc65)c4)n3)c2)cc1 ; N#Cc1ccc(-c2cccc(-c3cc(-c4cccc(-c5ccc(-c6cc(-c7ccccc7)nc(-c7ccccc7)n6)cc5)c4-c4ccc(-c5ccccc5)c5ccccc45)nc(-c4ccccc4)n3)c2)cc1]

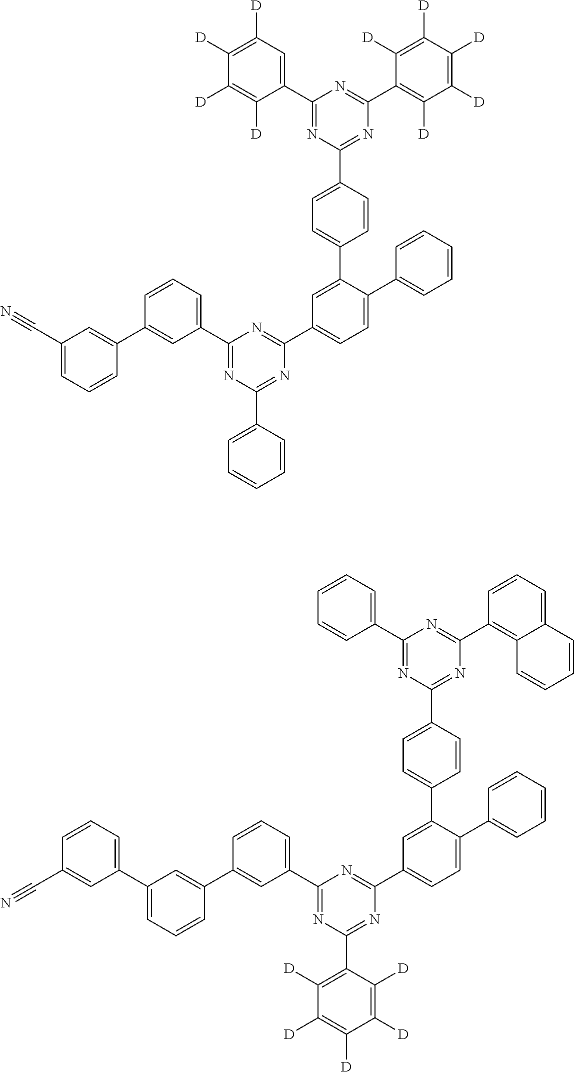 [SMILES: [2H]c1cc(-c2nc(-c3ccc(-c4cc(-c5nc(-c6ccccc6)nc(-c6cccc(-c7cccc(C#N)c7)c6)n5)ccc4-c4ccccc4)cc3)nc(-c3c([2H])c([2H])c([2H])c([2H])c3[2H])n2)c([2H])c([2H])c1[2H] ; [2H]c1c([2H])c([2H])c(-c2nc(-c3cccc(-c4cccc(-c5cccc(C#N)c5)c4)c3)nc(-c3ccc(-c4ccccc4)c(-c4ccc(-c5nc(-c6ccccc6)nc(-c6cccc7ccccc67)n5)cc4)c3)n2)c([2H])c1[2H]]

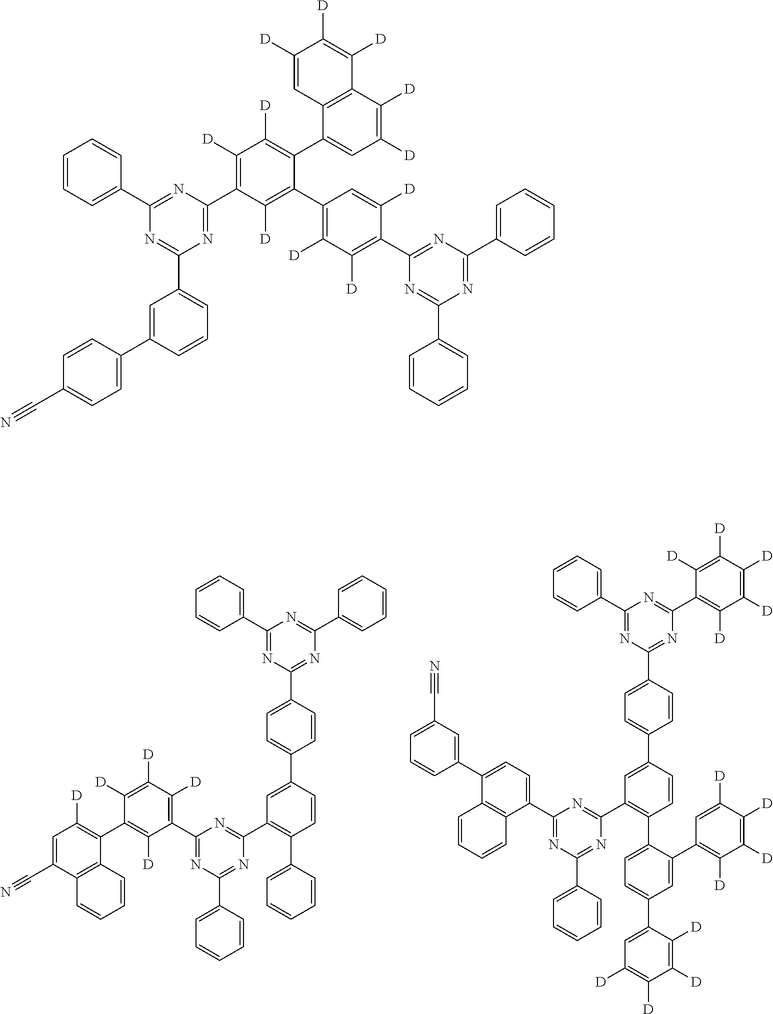

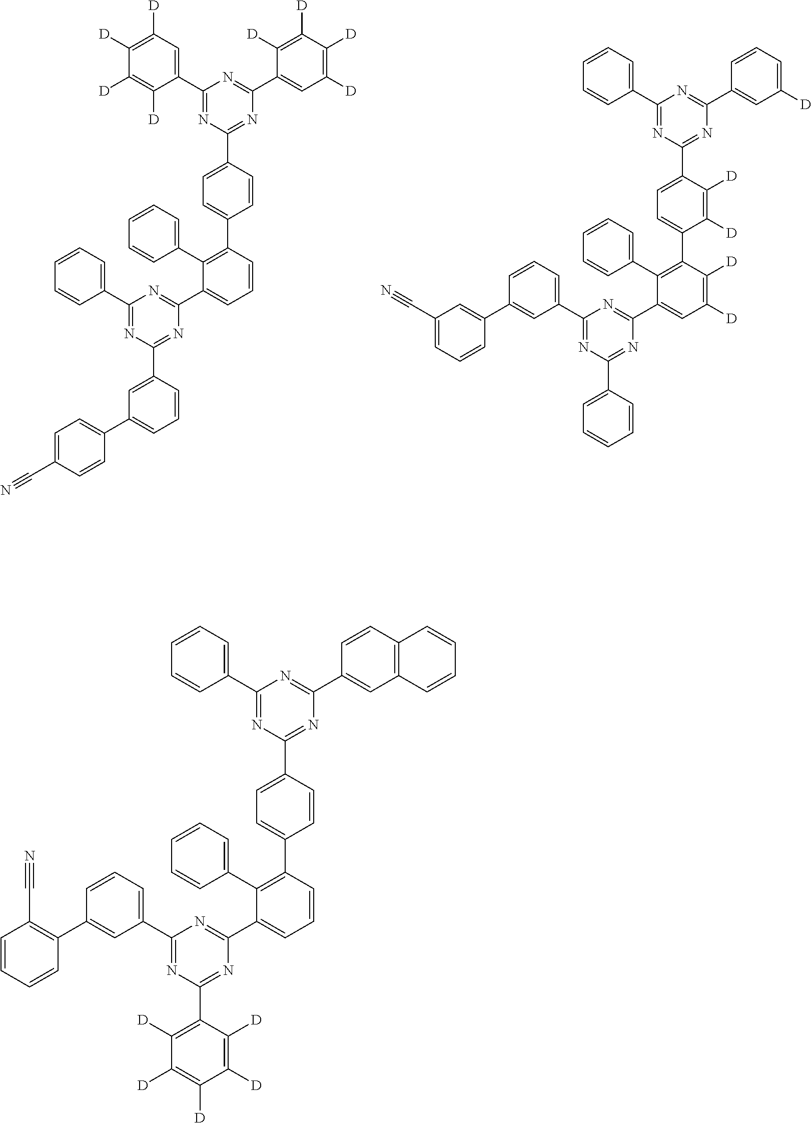 [SMILES: [2H]c1cc(-c2nc(-c3ccc(-c4cccc(-c5nc(-c6ccccc6)nc(-c6cccc(-c7ccc(C#N)cc7)c6)n5)c4-c4ccccc4)cc3)nc(-c3cc([2H])c([2H])c([2H])c3[2H])n2)c([2H])c([2H])c1[2H] ; [2H]c1cccc(-c2nc(-c3ccccc3)nc(-c3ccc(-c4c([2H])c([2H])cc(-c5nc(-c6ccccc6)nc(-c6cccc(-c7cccc(C#N)c7)c6)n5)c4-c4ccccc4)c([2H])c3[2H])n2)c1 ; [2H]c1c([2H])c([2H])c(-c2nc(-c3cccc(-c4ccccc4C#N)c3)nc(-c3cccc(-c4ccc(-c5nc(-c6ccccc6)nc(-c6ccc7ccccc7c6)n5)cc4)c3-c3ccccc3)n2)c([2H])c1[2H]]

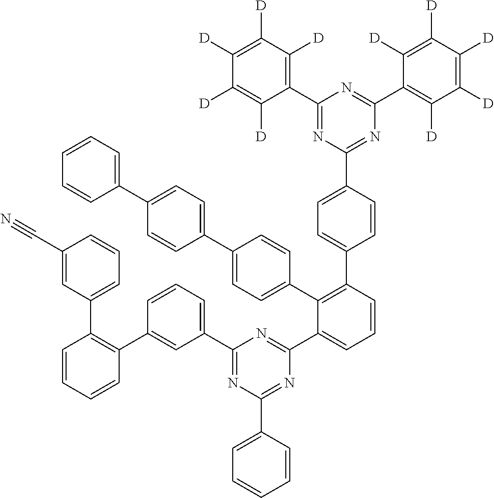 [SMILES: [2H]c1c([2H])c([2H])c(-c2nc(-c3ccc(-c4cccc(-c5nc(-c6ccccc6)nc(-c6cccc(-c7ccccc7-c7cccc(C#N)c7)c6)n5)c4-c4ccc(-c5ccc(-c6ccccc6)cc5)cc4)cc3)nc(-c3c([2H])c([2H])c([2H])c([2H])c3[2H])n2)c([2H])c1[2H]]

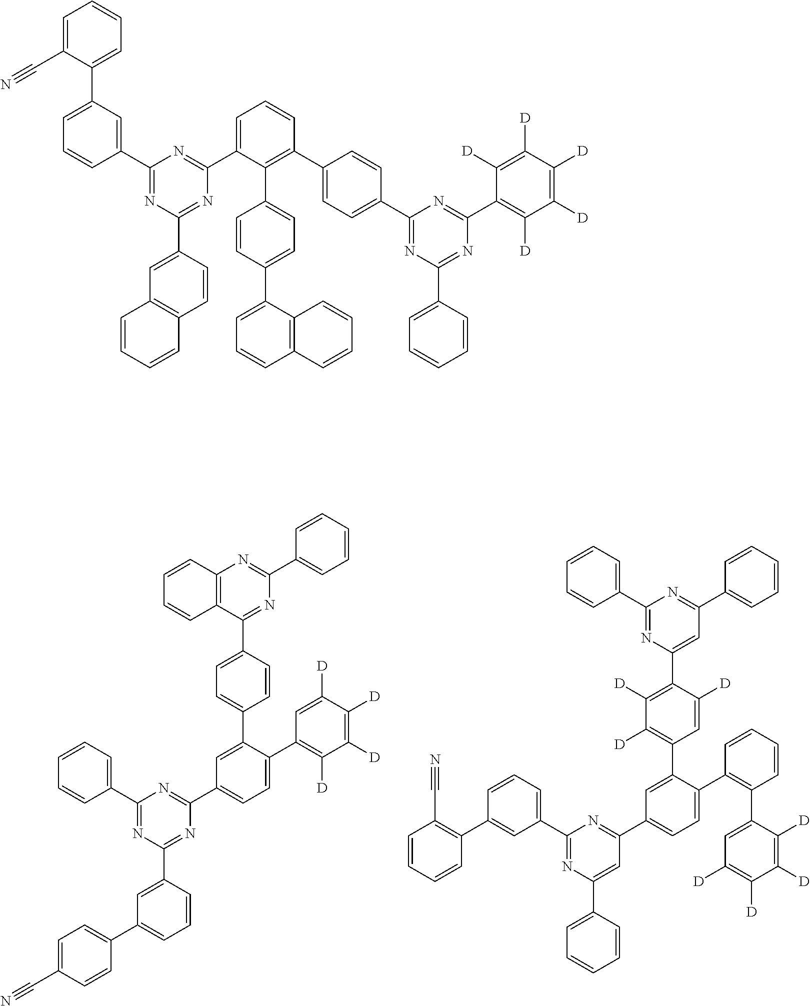 [SMILES: [2H]c1c([2H])c([2H])c(-c2nc(-c3ccccc3)nc(-c3ccc(-c4cccc(-c5nc(-c6cccc(-c7ccccc7C#N)c6)nc(-c6ccc7ccccc7c6)n5)c4-c4ccc(-c5cccc6ccccc56)cc4)cc3)n2)c([2H])c1[2H] ; [2H]c1cc(-c2ccc(-c3nc(-c4ccccc4)nc(-c4cccc(-c5ccc(C#N)cc5)c4)n3)cc2-c2ccc(-c3nc(-c4ccccc4)nc4ccccc34)cc2)c([2H])c([2H])c1[2H] ; [2H]c1cc(-c2ccccc2-c2ccc(-c3cc(-c4ccccc4)nc(-c4cccc(-c5ccccc5C#N)c4)n3)cc2-c2cc([2H])c(-c3cc(-c4ccccc4)nc(-c4ccccc4)n3)c([2H])c2[2H])c([2H])c([2H])c1[2H]]

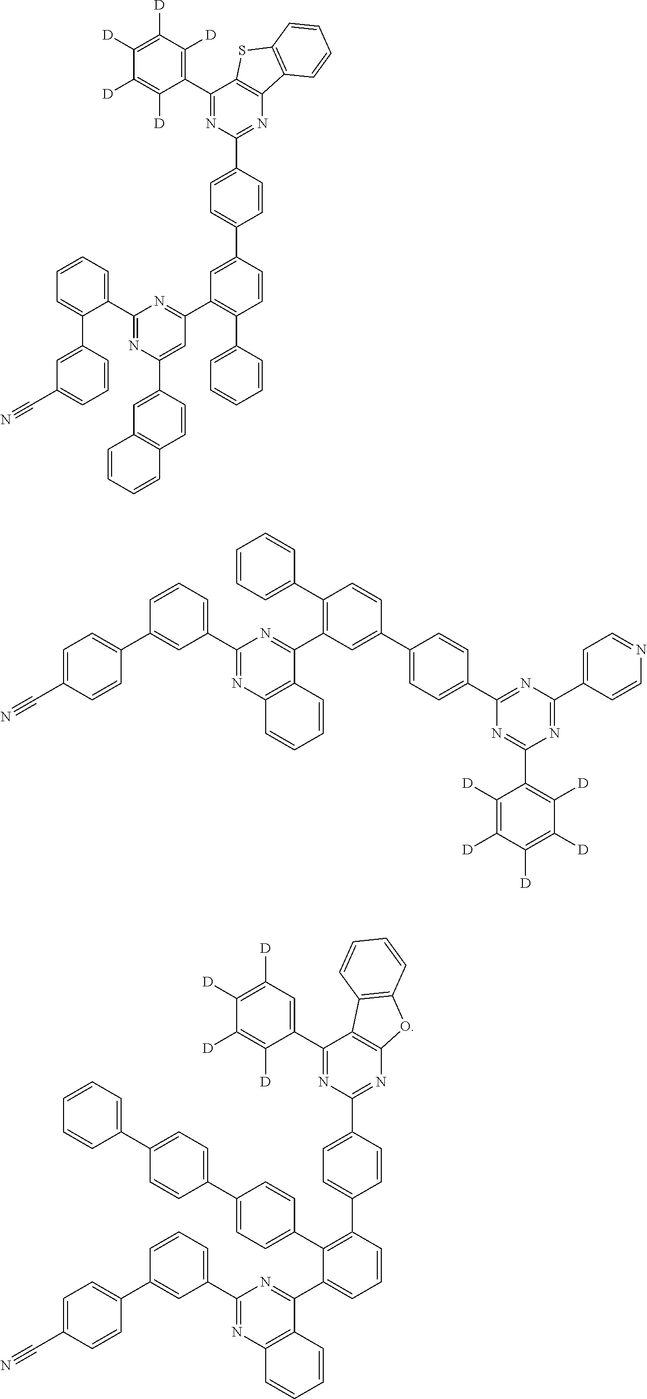 [SMILES: [2H]c1c([2H])c([2H])c(-c2nc(-c3ccc(-c4ccc(-c5ccccc5)c(-c5cc(-c6ccc7ccccc7c6)nc(-c6ccccc6-c6cccc(C#N)c6)n5)c4)cc3)nc3c2sc2ccccc23)c([2H])c1[2H] ; [2H]c1c([2H])c([2H])c(-c2nc(-c3ccncc3)nc(-c3ccc(-c4ccc(-c5ccccc5)c(-c5nc(-c6cccc(-c7ccc(C#N)cc7)c6)nc6ccccc56)c4)cc3)n2)c([2H])c1[2H] ; [2H]c1cc(-c2nc(-c3ccc(-c4cccc(-c5nc(-c6cccc(-c7ccc(C#N)cc7)c6)nc6ccccc56)c4-c4ccc(-c5ccc(-c6ccccc6)cc5)cc4)cc3)nc3oc4ccccc4c23)c([2H])c([2H])c1[2H]]

11. An organic light emitting device comprising:
a first electrode;
a second electrode; and
an organic material layer having one or more layers provided between the first electrode and the second electrode,
wherein one or more layers of the organic material layer comprise the compound of claim 1.

12. The organic light emitting device of claim 11, wherein the organic material layer comprises one or more layers of an electron transport layer, an electron injection layer, and an electron injection and transport layer, and one or more layers of the layers comprise the compound.
